(19) United States
(12) Patent Application Publication
(10) Pub. No.: US 2025/0270553 A1
(43) Pub. Date: Aug. 28, 2025

(54) STABILIZATION OF THERAPEUTIC TRANS-SPLICING RNA MOLECULES IN HUMAN CELLS

(71) Applicant: Tacit Therapeutics, Inc., South San Francisco, CA (US)

(72) Inventor: David Allen NELLES, South San Francisco, CA (US)

(73) Assignee: Tacit Therapeutics, Inc., South San Francisco, CA (US)

(21) Appl. No.: 18/858,155

(22) PCT Filed: Apr. 19, 2023
(86) PCT No.: PCT/US2023/065955

§ 371 Date: Oct. 18, 2024

Related U.S. Application Data
(60) Provisional application No. 63/332,914, filed on Apr. 20, 2022.

(51) Int. Cl. C12N 15/113

(52) U.S. Cl. CPC C12N 15/113; C12N 2310/11; C12N 2320/33; C12N 2320/51

(57) ABSTRACT

Disclosed are compositions comprising a nucleic acid molecule. The nucleic acid molecule may encode an exonic sequence or portion thereof of a target ribonucleic acid (RNA) sequence. The nucleic acid molecule may further encode one or more stabilization domains. The one or more stabilization domains may be configured to reduce a cellular nuclease activity compared to a nucleic acid molecule that does not comprise the one or more stabilization domains.

### CROSS-REFERENCE

This application claims the benefit of U.S. Provisional Application No. 63/332,914, filed Apr. 20, 2022, which is entirely incorporated herein by reference.

### STATEMENT AS TO FEDERALLY SPONSORED RESEARCH

This invention was made with government support under 2112383 awarded by National Science Foundation. The government has certain rights in the invention.

### INCORPORATION BY REFERENCE OF SEQUENCE LISTING

The present application is being filed along with a Sequence Listing in electronic format. The Sequence Listing is provided as a file entitled 63827-705601.XML, created Apr. 17, 2023, which is 96.1 kilobytes in size. The information in the electronic format of the Sequence Listing is incorporated by reference in its entirety.

### BACKGROUND

Many human diseases result from improper genetic sequences, including improper ribonucleic acid (RNA) sequences or improper deoxyribonucleic acid (DNA) sequences. To that end, effective treatment of human genetic disease necessitates efficient replacement of defective genetic sequences in human cells. RNA trans-splicing has been proposed as a human gene therapeutic but has not experienced success in clinical trials due to low RNA editing efficiency and therefore low efficacy. The efficiency of RNA trans-splicing may be defined as the fraction of a target RNA molecule that experiences a specific change in sequence composition that is mediated by trans-splicing. This efficiency measurement is a significant metric of therapeutic efficacy. One significant reason for inefficient trans-splicing is the short lifetime of therapeutic RNA molecules in human cells. Indeed, human cells express a variety of RNA exonucleases and endonucleases that rapidly degrade both cellular RNAs and therapeutic RNA such as RNA trans-splicing molecules.

### SUMMARY

Recognized herein is a long-felt but unmet need for the creation of long-lasting and efficacious treatments for human genetic diseases, including for treatments of diseases stemming, at least in part, from improper RNA sequences. The present disclosure describes improvements to RNA trans-splicing molecules that can address this long-felt but unmet need. The present disclosure provides compositions and methods for stabilization of trans-splicing RNA therapeutics in human cells. Specifically, the present disclosure provides compositions that increase the stability and therefore the efficacy of trans-splicing RNA molecules. The present disclosure also provides methods for replacement of chosen RNA sequences within target RNAs using stabilized RNA trans-splicing molecules to treat a disease in the context of a human gene therapy.

In certain aspects, described herein is a composition comprising a trans-splicing ribonucleic acid (RNA) comprising one or more stabilization domains that increase the trans-splicing efficiency of the trans-splicing RNA as compared to a trans-splicing RNA without one or more stabilization domains. In certain aspects, described herein is a composition comprising a trans-splicing ribonucleic acid (RNA), comprising: (a) one or more replacement domains that encode a therapeutic sequence operably linked to; (b) one or more intronic domains that promote RNA splicing of the replacement domain; (c) one or more antisense domains that promote binding to a target RNA molecule; and (d) one or more stabilization domains reduce the susceptibility of the trans-splicing RNA to nucleases as compared to a trans-splicing RNA without one or more stabilization domains. In some embodiments, the one or more stabilization domains comprise a chain of RNA nucleobases that form a tertiary structure. In some embodiments, the one or more stabilization domains comprise one or more exonuclease-resistant RNA sequences derived from a flavivirus genome. In some embodiments, the flavivirus genome is selected from the group consisting of: Yellow fever virus, Dengue virus, West Nile virus, and Zika virus. In some embodiments, the one or more stabilization domains comprise one or more exonuclease-resistant RNA sequences derived from a viral genome selected from the group consisting of: Kunjin virus, cell-fusing agent virus, tobacco etch virus, Montana myotis leukoencephalitis virus, and rhesus rhadinovirus. In some embodiments, the one or more stabilization domains comprise a chain of RNA nucleobases that form a stem-loop secondary structure. In some embodiments, the one or more stabilization domains comprise an RNA motif that forms a tertiary structure. In some embodiments, the tertiary structure comprises an RNA pseudoknot. In some embodiments, the tertiary structure comprises a guanosine quadruplex comprising at least one RNA motif containing 75% or more guanosine nucleobases. In some embodiments, the tertiary structure comprises an RNA triplex. In some embodiments, the triplex-forming sequence is derived from a human gene selected from the group consisting of: NEAT1 and MALAT1. In some embodiments, a stabilization domain of the one or more stabilization domains is less than 300 bases from the 3′ end of the trans-splicing nucleic acid. In some embodiments, a stabilization domain of the one or more stabilization domains is less than 300 bases from the 5′ end of the trans-splicing nucleic acid. In some embodiments, the trans-splicing RNA comprises 2 or more stabilization domains. In some embodiments, the composition comprises a 3′ untranslated region that further increases the stability of the trans-splicing RNA. In some embodiments, the composition comprises a 5′ untranslated region that further increases the stability of the trans-splicing RNA. In some embodiments, the composition comprises a replacement domain. In some embodiments, the replacement domain comprises a gene expression-enhancing element. In some embodiments, the stability-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, the composition comprises an RNA-binding protein that strengthens the interaction among the trans-splicing nucleic acid molecule and the target RNA molecule and increases trans-splicing efficiency In some embodiments, the trans-splicing RNA further comprises a heterologous promoter. In some embodiments, the composition comprises an engineered small nuclear RNA that promotes trans-splicing activity of the trans-splicing RNA. In some embodiments, described herein vector comprising the compositions described herein. In some embodiments, the vector is selected from the group consisting of: adeno-associated virus, retrovirus, lentivirus, adenovirus, nanoparticle, micelle, liposome, lipoplex, polymersome, polyplex, and dendrimer. In some embodiments, described herein is a cell comprising the vectors described herein. A method for treating a disease comprising administering to a patient in need of a therapeutically effective amount of a treatment comprising the trans-splicing nucleic acid molecule described herein, the vector described herein or the cell described herein. A method for correcting a genetic defect in a subject comprising administering to said subject the trans-splicing nucleic acid molecule described herein, the vector described herein or the cell described herein.

In certain aspects, described herein is a composition comprising a trans-splicing ribonucleic acid (RNA) comprising one or more stabilization domains configured to reduce a cellular nuclease activity compared to a trans-splicing RNA that does not comprise said one or more stabilization domains. In some embodiments, the one or more stabilization domains comprise a chain of RNA nucleobases that form a tertiary structure. In some embodiments, the one or more stabilization domains comprise one or more exonuclease-resistant RNA sequences derived from a flavivirus genome. In some embodiments, the flavivirus genome is selected from the group consisting of: Yellow fever virus, Dengue virus, West Nile virus, and Zika virus. In some embodiments, the one or more stabilization domains comprise one or more exonuclease-resistant RNA sequences derived from a viral genome selected from the group consisting of: Kunjin virus, cell-fusing agent virus, tobacco etch virus, Montana myotis leukoencephalitis virus, and rhesus rhadinovirus. In some embodiments, the one or more stabilization domains comprise a chain of RNA nucleobases that form a stem-loop secondary structure. In some embodiments, the one or more stabilization domains comprise an RNA motif that forms a tertiary structure. In some embodiments, the tertiary structure comprises an RNA pseudoknot. In some embodiments, the tertiary structure comprises a guanosine quadruplex comprising at least one RNA motif containing 75% or more guanosine nucleobases. In some embodiments, the tertiary structure comprises an RNA triplex. In some embodiments, a sequence forming the RNA triplex is derived from a human gene selected from the group consisting of: NEAT1 and MALAT1. In some embodiments, a stabilization domain of the one or more stabilization domains is less than 300 bases from the 3′ end of the trans-splicing nucleic acid. In some embodiments, a stabilization domain of the one or more stabilization domains is less than 300 bases from the 5′ end of the trans-splicing nucleic acid. In some embodiments, the trans-splicing RNA comprises 2 or more stabilization domains. In some embodiments, the composition further comprises a 3′ untranslated region that further increases the stability of the trans-splicing RNA. In some embodiments, the composition further comprises a 5′ untranslated region that further increases the stability of the trans-splicing RNA. In some embodiments, the composition further comprises a replacement domain. In some embodiments, the replacement domain comprises a gene expression-enhancing element. In some embodiments, the stability-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, the composition further comprises an RNA-binding protein that strengthens the interaction among the trans-splicing nucleic acid molecule and the target RNA molecule and increases trans-splicing efficiency In some embodiments, the trans-splicing RNA further comprises a heterologous promoter. In some embodiments, the composition further comprises an engineered small nuclear RNA that promotes trans-splicing activity of the trans-splicing RNA. In some embodiments, described herein is a vector comprising the composition described herein. In some embodiments, the vector is selected from the group consisting of: adeno-associated virus, retrovirus, lentivirus, adenovirus, nanoparticle, micelle, liposome, lipoplex, polymersome, polyplex, and dendrimer. In some embodiments, described herein is a cell comprising the vector described herein. In some embodiments, described herein is a method for treating a disease comprising administering to a patient in need of a therapeutically effective amount of a treatment comprising the composition described herein, the vector described herein, or the cell described herein. In some embodiments, described herein is a method for correcting a genetic defect in a subject comprising administering to said subject the composition described herein, the vector described herein, or the cell described herein.

In certain aspects, described herein is a composition comprising a nucleic acid molecule, wherein said nucleic acid molecule encodes (i) an exonic sequence or portion thereof of a target ribonucleic acid (RNA) sequence and (ii) one or more stabilization domains configured to reduce a susceptibility of said exonic sequence or portion thereof to degradation as compared to a degradation of an exonic sequence or portion thereof in a nucleic acid molecule lacking said one or more stabilization domains. In some embodiments, the nucleic acid comprises RNA. In some embodiments, the nucleic acid comprises deoxyribonucleic acid (DNA). In some embodiments, the nucleic acid comprises a DNA/RNA hybrid. In some embodiments, the nucleic acid comprises a nucleic acid analog. In some embodiments, the nucleic acid comprises a chemically-modified nucleic acid. In some embodiments, the nucleic acid is a chimera comprising two or more nucleic acids or nucleic acid analogs. In some embodiments, the one or more stabilization domains comprise a chain of RNA nucleobases that form a tertiary structure. In some embodiments, the one or more stabilization domains comprise one or more exonuclease-resistant RNA sequences derived from a flavivirus genome. In some embodiments, the flavivirus genome is selected from the group consisting of: Yellow fever virus, Dengue virus, West Nile virus, and Zika virus. In some embodiments, the one or more stabilization domains comprise one or more exonuclease-resistant nucleic acid sequences derived from a viral genome selected from the group consisting of: Kunjin virus, cell-fusing agent virus, tobacco etch virus, Montana myotis leukoencephalitis virus, and rhesus rhadinovirus. In some embodiments, the one or more stabilization domains comprise a chain of RNA nucleobases that form a stem-loop secondary structure. In some embodiments, the one or more stabilization domains comprise an RNA motif that forms a tertiary structure. In some embodiments, the tertiary structure comprises an RNA pseudoknot. In some embodiments, the tertiary structure comprises a guanosine quadruplex comprising at least one RNA motif containing 75% or more guanosine nucleobases. In some embodiments, the tertiary structure comprises an RNA triplex. In some embodiments, a sequence forming the RNA triplex is derived from a human gene selected from the group consisting of: NEAT1 and MALAT1. In some embodiments, a stabilization domain of the one or more stabilization domains is less than 300 bases from the 3′ end of the trans-splicing nucleic acid. In some embodiments, a stabilization domain of the one or more stabilization domains is less than 300 bases from the 5′ end of the trans-splicing nucleic acid. In some embodiments, the nucleic acid comprises 2 or more stabilization domains. In some embodiments, the composition further comprises a 3′ untranslated region that further increases the stability of the nucleic acid. In some embodiments, the composition further comprises a 5′ untranslated region that further increases the stability of the nucleic acid. In some embodiments, the composition further comprises a replacement domain. In some embodiments, the replacement domain comprises a gene expression-enhancing element. In some embodiments, the stability-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, the composition further comprises an RNA-binding protein that strengthens the interaction among the nucleic acid molecule and a target RNA molecule and increases trans-splicing efficiency. In some embodiments, the nucleic acid further comprises a heterologous promoter. In some embodiments, the composition further comprises an engineered small nuclear RNA that promotes trans-splicing activity of the nucleic acid. In some embodiments, described herein is a vector comprising the composition described herein. In some embodiments, the vector is selected from the group consisting of: adeno-associated virus, retrovirus, lentivirus, adenovirus, nanoparticle, micelle, liposome, lipoplex, polymersome, polyplex, and dendrimer. In some embodiments, described herein is a cell comprising the vector described herein. In some embodiments, described herein is a method for treating a disease comprising administering to a patient in need of a therapeutically effective amount of a treatment comprising the composition described herein, the vector described herein, or the cell described herein. In some embodiments, described herein is a method for correcting a genetic defect in a subject comprising administering to said subject the composition described herein, the vector described herein, or the cell described herein.

In certain aspects, described herein is a composition comprising a nucleic acid molecule, wherein said nucleic acid molecule encodes (i) an exonic sequence or portion thereof of a target ribonucleic acid (RNA) sequence and (ii) one or more stabilization domains configured to increase a trans-splicing efficiency of said exonic sequence or portion thereof relative to said exonic sequence of a target RNA that is not administered a stabilization domain. In some embodiments, the nucleic acid is RNA, deoxyribonucleic acid (DNA), a DNA/RNA hybrid, a nucleic acid analog, a chemically-modified nucleic acid, or a chimera composed of two or more nucleic acids or nucleic acid analogs. In some embodiments, the nucleic acid comprises RNA. In some embodiments, the nucleic acid comprises DNA. In some embodiments, the nucleic acid comprises a DNA/RNA hybrid. In some embodiments, the nucleic acid comprises a nucleic acid analog. In some embodiments, the nucleic acid comprises a chemically-modified nucleic acid. In some embodiments, the nucleic acid is a chimera comprising two or more nucleic acids or nucleic acid analogs. In some embodiments, the one or more stabilization domains comprise a chain of RNA nucleobases that form a tertiary structure. In some embodiments, the one or more stabilization domains comprise one or more exonuclease-resistant RNA sequences derived from a flavivirus genome. In some embodiments, the flavivirus genome is selected from the group consisting of: Yellow fever virus, Dengue virus, West Nile virus, and Zika virus. In some embodiments, the one or more stabilization domains comprise one or more exonuclease-resistant nucleic acid sequences derived from a viral genome selected from the group consisting of: Kunjin virus, cell-fusing agent virus, tobacco etch virus, Montana myotis lenkoencephalitis virus, and rhesus rhadinovirus. In some embodiments, the one or more stabilization domains comprise a chain of RNA nucleobases that form a stem-loop secondary structure. In some embodiments, the one or more stabilization domains comprise an RNA motif that forms a tertiary structure. In some embodiments, the tertiary structure comprises an RNA pseudoknot. In some embodiments, the tertiary structure comprises a guanosine quadruplex comprising at least one RNA motif containing 75% or more guanosine nucleobases. In some embodiments, the tertiary structure comprises an RNA triplex. In some embodiments, a sequence forming the RNA triplex is derived from a human gene selected from the group consisting of: NEAT1 and MALAT1. In some embodiments, a stabilization domain of the one or more stabilization domains is less than 300 bases from the 3′ end of the trans-splicing nucleic acid. In some embodiments, a stabilization domain of the one or more stabilization domains is less than 300 bases from the 5′ end of the trans-splicing nucleic acid. In some embodiments, the nucleic acid comprises 2 or more stabilization domains. In some embodiments, the composition further comprises a 3′ untranslated region that further increases the stability of the nucleic acid. In some embodiments, the composition further comprises a 5′ untranslated region that further increases the stability of the nucleic acid. In some embodiments, the target RNA comprises a mutation that is targeted by the exonic sequence of portion thereof. In some embodiments, the exonic sequence of portion thereof comprises a gene expression-enhancing element. In some embodiments, the stability-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, the composition further comprises an RNA-binding protein that strengthens the interaction among the nucleic acid molecule and a target RNA molecule and increases trans-splicing efficiency. In some embodiments, the nucleic acid further comprises a heterologous promoter. In some embodiments, the composition further comprises an engineered small nuclear RNA that promotes trans-splicing activity of the nucleic acid. In some embodiments, described herein is a vector comprising the composition described herein. In some embodiments, the vector is selected from the group consisting of: adeno-associated virus, retrovirus, lentivirus, adenovirus, nanoparticle, micelle, liposome, lipoplex, polymersome, polyplex, and dendrimer. In some embodiments, described herein is a cell comprising the vector described herein. In some embodiments, described herein is a method for treating a disease comprising administering to a patient in need of a therapeutically effective amount of a treatment comprising the composition described herein, the vector described herein, or the cell described herein. In some embodiments, described herein is a method for correcting a genetic defect in a subject comprising administering to said subject the composition described herein, the vector described herein, or the cell described herein.

In certain aspects, described herein is a composition comprising a nucleic acid molecule, wherein said nucleic acid molecule encodes (i) an exonic sequence or portion thereof of a target ribonucleic acid (RNA) sequence and (ii) one or more stabilization domains configured to reduce a cellular nuclease activity compared to a nucleic acid molecule that does not comprise said one or more stabilization domains. In some embodiments, the nucleic acid is RNA, deoxyribonucleic acid (DNA), a DNA/RNA hybrid, a nucleic acid analog, a chemically-modified nucleic acid, or a chimera composed of two or more nucleic acids or nucleic acid analogs. In some embodiments, the nucleic acid comprises RNA. In some embodiments, the nucleic acid comprises DNA. In some embodiments, the nucleic acid comprises a DNA/RNA hybrid. In some embodiments, the nucleic acid comprises a nucleic acid analog. In some embodiments, the nucleic acid comprises a chemically-modified nucleic acid. In some embodiments, the nucleic acid is a chimera comprising two or more nucleic acids or nucleic acid analogs. In some embodiments, the one or more stabilization domains comprise a chain of RNA nucleobases that form a tertiary structure. In some embodiments, the one or more stabilization domains comprise one or more exonuclease-resistant RNA sequences derived from a flavivirus genome. In some embodiments, the flavivirus genome is selected from the group consisting of: Yellow fever virus, Dengue virus, West Nile virus, and Zika virus. In some embodiments, the one or more stabilization domains comprise one or more exonuclease-resistant nucleic acid sequences derived from a viral genome selected from the group consisting of: Kunjin virus, cell-fusing agent virus, tobacco etch virus, Montana myotis leukoencephalitis virus, and rhesus rhadinovirus. In some embodiments, the one or more stabilization domains comprise a chain of RNA nucleobases that form a stem-loop secondary structure. In some embodiments, the one or more stabilization domains comprise an RNA motif that forms a tertiary structure. In some embodiments, the tertiary structure comprises an RNA pseudoknot. In some embodiments, the tertiary structure comprises a guanosine quadruplex comprising at least one RNA motif containing 75% or more guanosine nucleobases. In some embodiments, the tertiary structure comprises an RNA triplex. In some embodiments, a sequence forming the RNA triplex is derived from a human gene selected from the group consisting of: NEAT1 and MALAT1. In some embodiments, a stabilization domain of the one or more stabilization domains is less than 300 bases from the 3′ end of the trans-splicing nucleic acid. In some embodiments, a stabilization domain of the one or more stabilization domains is less than 300 bases from the 5′ end of the trans-splicing nucleic acid. In some embodiments, the nucleic acid comprises 2 or more stabilization domains. In some embodiments, the composition further comprises a 3′ untranslated region that further increases the stability of the nucleic acid. In some embodiments, the composition further comprises a 5′ untranslated region that further increases the stability of the nucleic acid. In some embodiments, the target RNA comprises a mutation that is targeted by the exonic sequence of portion thereof. In some embodiments, the exonic sequence of portion thereof comprises a gene expression-enhancing element. In some embodiments, the stability-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, the composition further comprises an RNA-binding protein that strengthens the interaction among the nucleic acid molecule and a target RNA molecule and increases trans-splicing efficiency. In some embodiments, the nucleic acid further comprises a heterologous promoter. In some embodiments, the composition further comprises an engineered small nuclear RNA that promotes trans-splicing activity of the nucleic acid. In some embodiments, described herein is a vector comprising the composition described herein. In some embodiments, the vector is selected from the group consisting of: adeno-associated virus, retrovirus, lentivirus, adenovirus, nanoparticle, micelle, liposome, lipoplex, polymersome, polyplex, and dendrimer. In some embodiments, described herein is a cell comprising the vector described herein. In some embodiments, described herein is a method for treating a disease comprising administering to a patient in need of a therapeutically effective amount of a treatment comprising the composition described herein, the vector described herein, or the cell described herein. In some embodiments, described herein is a method for correcting a genetic defect in a subject comprising administering to said subject the composition described herein, the vector described herein, or the cell described herein.

In certain aspects, described herein is a method for treating a disease comprising administering a nucleic acid molecule, wherein said nucleic acid molecule encodes (i) an exonic sequence or portion thereof of a target ribonucleic acid (RNA) sequence and (ii) one or more stabilization domains configured to reduce a susceptibility of said exonic sequence or portion thereof to degradation as compared to a degradation of an exonic sequence or portion thereof in a nucleic acid molecule lacking said one or more stabilization domains. In certain aspects, described herein is a method for treating a disease comprising administering a nucleic acid molecule, wherein said nucleic acid molecule encodes (i) an exonic sequence or portion thereof of a target ribonucleic acid (RNA) sequence and (ii) one or more stabilization domains configured to reduce a cellular nuclease activity compared to a nucleic acid molecule that does not comprise said one or more stabilization domains.

### INCORPORATION BY REFERENCE

All publications, patents, and patent applications mentioned in this specification are herein incorporated by reference to the same extent as if each individual publication, patent, or patent application was specifically and individually indicated to be incorporated by reference. Further, to the extent that any meaning or definition of a term in this document conflicts with any meaning or definition of the same term in a document incorporated by reference, the meaning or definition assigned to that term in this document shall govern.

### BRIEF DESCRIPTION OF THE DRAWINGS

The patent or application file contains at least one drawing executed in color. Copies of this patent or patent application publication with color drawing(s) will be provided by the U.S. Patent and Trademark Office upon request and payment of the necessary fee.

FIG. 1 illustrates the unmet need addressed by the systems and methods described herein and provides a schematic of said systems and methods. FIG. 1A illustrates an exemplary concept of human genetic disease where mutated (“defective”) DNA sequences are transcribed into RNA which directly contribute to disease (“RNA pathogenicity”) or are translated into disease-causing protein (“translation of pathogenic protein”). FIG. 1B illustrates an exemplary concept of RNA trans-splicing technology where a mutation-carrying RNA molecule is targeted by a trans-splicing RNA that corrects the mutation. State-of-the-art trans-splicing RNAs generate RNA correction at levels insufficient to halt or reverse progression of disease. FIG. 1C further illustrates exemplary state-of-the-art trans-splicing technology where cellular nucleases degrade the trans-splicing molecule which results in lower trans-splicing efficiency. Specifically, the levels of “corrected RNA” are insufficient to reverse or halt disease progression. FIG. 1D illustrates an exemplary concept of stabilizing sequences in the context of trans-splicing. Specifically, the addition of RNA stabilizing sequences to the trans-splicing RNA may increase the level of trans-splicing RNA, and therefore may increase the efficiency of the trans-splicing reaction and therefore also increase the levels of “corrected RNA”. This efficiency increase may be sufficient to halt or reverse disease progression and/or eliminate key disease phenotypes, thereby providing an effective therapeutic for human genetic disease.

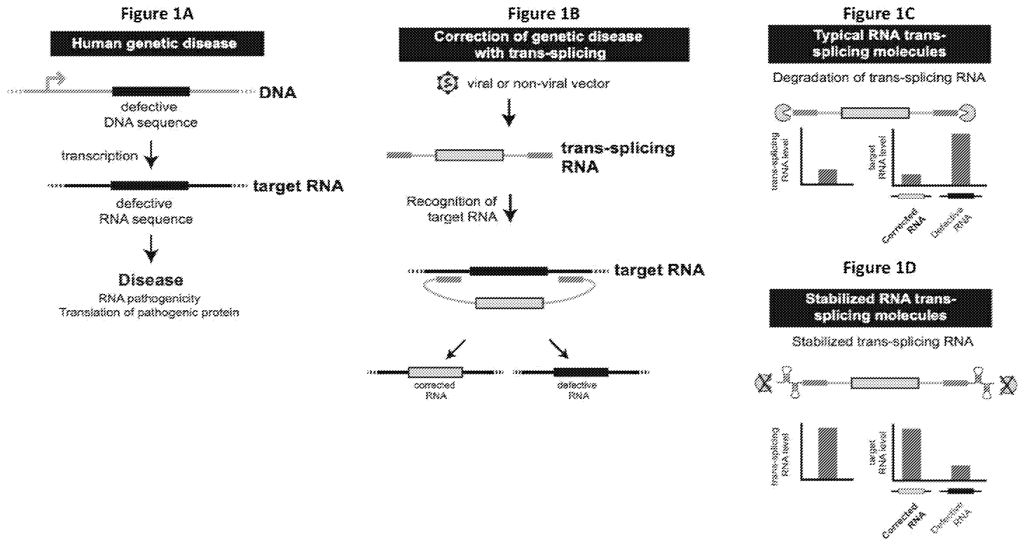

FIG. 2 illustrates three exemplary embodiments of the stabilized trans-splicing RNA. FIG. 2A describes a double trans-splicing molecule which carries two Antisense Domains, one Replacement Domain, two Intronic Domains, and at least one Stabilizing Domain at the 5′ and/or 3′ end of the trans-splicing molecule. This design promotes replacement of an internal sequence within the target RNA while maintaining the adjacent 5′ and 3′ sequences around the replaced sequence. FIGS. 2B and 2C describe terminal trans-splicing molecules that both contain one Antisense Domain, one Replacement Domain, one Intronic Domain, and at least one Stabilizing Domain at the 5′ and/or 3′ end of the trans-splicing molecule. FIG. 2B illustrates the design of a 3′ terminal trans-splicing RNA that will replace a 3′ terminal end of a target RNA while maintaining a 5′ end. FIG. 2C illustrates the design of a 5′ terminal trans-splicing molecule that will replace a 5′ terminal end of a target RNA while maintaining a 3′ end.

FIG. 3 illustrates an experiment designed to reveal the effect of stabilizing sequences in the context of internal trans-splicing via production of green fluorescence protein (GFP). FIG. 3A illustrates the design of a split GFP reporter that carries N- and C-terminal portions of GFP (“N-GFP” and “° C.-GFP”) but lacks an internal GFP sequence required for fluorescence. In the reporter, this internal sequence is replaced by a short exon with a stop codon that is flanked by introns. The internal sequence (“int-GFP”) is the replacement sequence within an RNA trans-splicing molecule that is flanked by two intronic sequences, two antisense sequences, and one or more terminal stabilizing sequences (“3′-end stabilized trans-splicing RNA” and “5′-end stabilized trans-splicing RNA”). FIG. 3B illustrates the activity of the reporter alone so that cis-splicing produces a GFP sequence interrupted by a stop codon therefore producing no GFP signal. FIG. 3C illustrates the activity of the reporter in the presence of the trans-splicing molecule without inclusion of stabilizing sequences in the trans-splicing molecule. Without stabilizing sequences, the exonic sequence to be trans-spliced into the target RNA is prone to degradation, e.g., via cellular exonucleases. As a result, trans-splicing is less likely to be successfully. Resultantly, as in the scenario in FIG. 3A, where no trans-splicing molecule is used, in FIG. 3B, cis-splicing occurs primarily and GFP signal is not efficiently generated. FIG. 3D illustrates the activity of the reporter in the presence of the trans-splicing molecule with inclusion of stabilizing sequences so that trans-splicing occurs primarily and GFP signal is efficiently produced. This is because, with the inclusion of stabilizing sequences, the exonic sequence to be trans-spliced into the target RNA is less prone to degradation, e.g., via cellular exonucleases. Therefore, more of the trans-splicing RNA is preserved, and trans-splicing is likelier to be successful.

FIG. 4 illustrates an experiment designed to reveal the effect of stabilizing sequences in the context of 5′ terminal trans-splicing. FIG. 4A illustrates the design of a split GFP reporter that carries a C-terminal portion of GFP (“C-GFP”) but lacks an N-terminal GFP sequence required for fluorescence. In the reporter, this N-terminal GFP sequence is replaced by a short exon with a stop codon that is flanked by introns. The N-terminal sequence (“N-GFP”) is the replacement sequence within an RNA trans-splicing molecule that is flanked by one intronic sequence, one antisense sequence, and one or more terminal stabilizing sequences (“3′-end stabilized trans-splicing RNA” and “5′-end stabilized trans-splicing RNA”). FIG. 4B illustrates the activity of the reporter alone so that cis-splicing produces a GFP sequence interrupted by a stop codon therefore producing no GFP signal. FIG. 4C illustrates the activity of the reporter in the presence of the trans-splicing molecule without inclusion of stabilizing sequences in the trans-splicing molecule. Without stabilizing sequences, the exonic sequence to be trans-spliced into the target RNA is prone to degradation, e.g., via cellular exonucleases. As a result, trans-splicing is less likely to be successfully. Resultantly, as in the scenario in FIG. 4A, where no trans-splicing molecule is used, in FIG. 4B cis-splicing occurs primarily and GFP signal is not efficiently produced. FIG. 4D illustrates the activity of the reporter in the presence of the trans-splicing molecule with inclusion of stabilizing sequences so that trans-splicing occurs primarily and GFP signal is efficiently produced. This is because, with the inclusion of stabilizing sequences, the exonic sequence to be trans-spliced into the target RNA is less prone to degradation, e.g., via cellular exonucleases. Therefore, more of the trans-splicing RNA is preserved, and trans-splicing is more likely to be successful.

FIG. 5 illustrates an experiment designed to reveal the effect of stabilizing sequences in the context of 3′ terminal trans-splicing. FIGURE SA illustrates the design of a split GFP reporter that carries a N-terminal portion of GFP (“N-GFP”) but lacks an C-terminal GFP sequence required for fluorescence. In the reporter, this C-terminal GFP sequence is replaced by a short exon with a stop codon that is flanked by introns. The C-terminal sequence (“C-GFP”) is the replacement sequence within an RNA trans-splicing molecule that is flanked by one intronic sequence, one antisense sequence, and one or more terminal stabilizing sequences. FIG. 5B illustrates the activity of the reporter alone so that cis-splicing produces a GFP sequence interrupted by a stop codon therefore producing no GFP signal. FIG. 5C illustrates the activity of the reporter in the presence of the trans-splicing molecule without inclusion stabilizing sequences in the trans-splicing molecule. Without stabilizing sequences, the exonic sequence to be trans-spliced into the target RNA is prone to degradation, e.g., via cellular exonucleases. As a result, trans-splicing is less likely to be successfully. Resultantly, as in the scenario in FIG. 3A, where no trans-splicing molecule is used, in FIG. 3B, cis-splicing occurs primarily and GFP signal is not efficiently produced. FIG. 5D illustrates the activity of the reporter in the presence of the trans-splicing molecule with inclusion of stabilizing sequences so that trans-splicing occurs primarily and GFP signal is produced. This is because, with the inclusion of stabilizing sequences, the exonic sequence to be trans-spliced into the target RNA is less prone to degradation, e.g., via cellular exonucleases. Therefore, more of the trans-splicing RNA is preserved, and trans-splicing is likelier to be successful.

FIG. 6 is the result of an experiment conducted to assess the influence of stabilizing sequences on trans-splicing efficiency.

### DETAILED DESCRIPTION

The present disclosure provides a nucleic acid molecule comprising a stabilizing sequence. The nucleic acid molecule may comprise a ribonucleic acid (RNA), a deoxyribonucleic acid (DNA), or any combination thereof. In some embodiments, an RNA molecule carries stabilizing sequences that selectively binds and promotes a trans-splicing reaction with a target RNA molecule. In some embodiments, the RNA molecule carries a Replacement Domain that corresponds to a mutated or missing sequence in a target RNA. In some embodiments, an DNA molecule carries stabilizing sequences. In some embodiments, the stabilizing sequences carried by the DNA molecule encode RNA stabilizing sequences. In some embodiments, the DNA molecule encodes a gene or portion thereof to be transcribed. In some embodiments, the gene or portion thereof encodes a Replacement Domain. In some embodiments, the Replacement Domain corresponds to a mutated or missing sequence in a target RNA. In some embodiments, the DNA molecule is transcribed into a messenger RNA molecule, and the messenger RNA molecule then selectively binds and promotes a trans-splicing reaction with a target RNA. The present disclosure provides vectors, compositions and cells comprising or encoding the trans-splicing nucleic acid molecule. The present disclosure provides methods of using the trans-splicing RNA molecule, vectors, compositions and cells of the present disclosure to treat a disease or disorder.

An aspect of the present disclosure provides a composition comprising a trans-splicing RNA molecule comprising (a) at least one domain that promotes trans-splicing (“Intronic Domain”), (b) at least one binding domain (“Antisense Domain”) that contains or consists of a sequence complementary to a pre-mRNA present in a human cells (“Target RNA”), (c) a coding domain that is inserted into the Target RNA via trans-splicing (“Replacement Domain”), and (d) a stabilizing sequence (“Stabilizing Domain”) that protects the trans-splicing molecule from degradation. The Stabilizing Domain blocks or attenuates that activity of nuclease enzymes to increase the effective level of the trans-splicing molecule in human cells and therefore increase the overall efficiency of RNA trans-splicing. In other embodiments, the present disclosure provides a composition comprising a nucleic acid sequence encoding the trans-splicing RNA molecule.

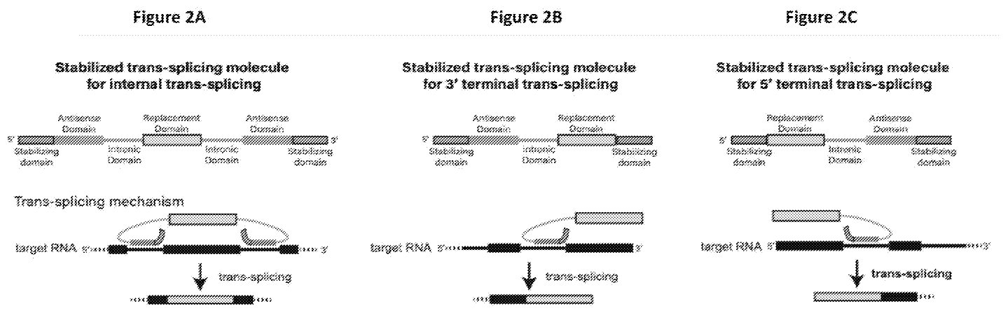

In one aspect, the present disclosure provides a trans-splicing RNA molecule comprising four types of domains (FIG. 2). In a second aspect, the present disclosure provides a trans-splicing DNA molecule comprising four types of domains. In some embodiments, the trans-splicing DNA comprises a gene or portion thereof to be transcribed. In some embodiments, the gene or portion thereof corresponds to a missing sequence in a target RNA. In some embodiments, the DNA molecule is transcribed into a messenger RNA molecule, and the messenger RNA molecule then selectively binds and promotes a trans-splicing reaction with a target RNA. In some embodiments, one of the three domain types is the Replacement Domain which is inserted into a Target RNA molecule via a trans-splicing reaction. In some embodiments, the Replacement Domain comprises an exonic sequence. In some embodiments, a DNA molecule comprises a gene or portion thereof encoding the Replacement Domain described herein. In some embodiments, an RNA molecule comprises the Replacement Domain described herein. In some embodiments, a second domain type is the Antisense Domain which is complementary to a Target RNA. In some embodiments, a DNA molecule comprises an. Antisense Domain described herein. In some embodiments, an RNA molecule comprises an Antisense Domain described herein. In some embodiments, a third domain type is the Intronic Domain which promotes the trans-splicing reaction between the trans-splicing RNA molecule and the Target RNA. The Intronic Domain may comprise RNA. The Intronic Domain may comprise DNA. The Intronic Domain comprising DNA may be transcribed into an Intronic Domain comprising RNA. In some embodiments, an DNA molecule comprises an Intronic Domain described herein. In some embodiments, an RNA molecule comprises an Intronic Domain described herein. In some embodiments, the Intronic Domain promotes the trans-splicing reaction between the trans-splicing DNA molecule and the target RNA. In some embodiments, the fourth domain is a Stabilizing Domain that carries sequences that block the activity of cellular nucleases. In some embodiments, a DNA molecule comprises a Stabilizing Domain described herein. In some embodiments, an RNA molecule comprises a Stabilizing Domain described herein. The Stabilizing Domain may comprise DNA or RNA. A Stabilizing Domain comprising DNA may encode a Stabilizing Domain comprising RNA. Blocking the activity of cellular nucleases may increase the effective level of the trans-splicing molecule in human cells. This combination of trans-splicing domains (Replacement, Intronic, and Antisense Domains) with the Stabilizing Domain promotes RNA trans-splicing in a manner that is sufficient to replace disease-causing RNA sequences in human cells to address disease. Indeed, low efficiency has been a major barrier to many nucleic acid editing approaches including trans-splicing. The present disclosure provides compositions and methods for specifically targeting disease-causing RNA molecules and replacing disease-causing RNA sequences within these RNA molecules with high efficiency. The trans-splicing RNA molecule implementations show utility in a variety of contexts including replacement of disease-causing sequences or insertion of engineered sequences into Target RNAs.

In some embodiments, the nucleic acid described herein comprises a Localization Domain, an Antisense Domain, an Intronic Domain, and a Replacement Domain. In some embodiments, the nucleic acid comprises at least two Localization Domains. In some embodiments, the nucleic acid comprises at least two Antisense Domains. In some embodiments, the nucleic acid comprises at least two Intronic Domains. In some embodiments, the nucleic acid described herein comprises in order from the 5′ end to the 3′ end a Localization Domain, an Antisense Domain, an Intronic Domain, and a Replacement domain. In some embodiments, the nucleic acid described herein comprises in order from the 5′ end to the 3′ end a Replacement Domain, an Intronic Domain, an Antisense Domain, and a Localization Domain. In some embodiments, the nucleic acid described herein comprises in order from the 5′ end to the 3′ end a first Localization Domain, a first Antisense Domain, a first Intronic Domain, a Replacement Domain, a second Intronic Domain, a second Antisense Domain, and a second Localization Domain. In some embodiments, the nucleic acid further comprises at least one Stabilization Domain. In some embodiments, the at least one Stabilization Domain is located at the 5′ end or the 3′ end of the nucleic acid,

The engineered sequences can alter the translation or stability of Target RNAs to increase or decrease protein production or Target RNA levels. The engineered sequences (e.g., polynucleotide sequences) disclosed herein may be codon-optimized. Codon optimization refers to the fact that different cells differ in their usage of particular codons. This codon bias corresponds to a bias in the relative abundance of particular tRNAs in the cell type. By altering the codons in the sequence to match with the relative abundance of corresponding tRNAs, it is possible to increase expression. In some instances, it is also possible to decrease expression by deliberately choosing codons for which the corresponding (RNAs are rare in a particular cell type.

In some instances, DNA encodes an exonic or replacement sequence that can be trans-spliced into RNA in order to modify (e.g., fix) the sequence. In some instances, the modification or fixing of the RNA sequence via trans-splicing increases protein production. This disclosure provides vectors, compositions and cells comprising or encoding the trans-splicing RNA and methods of using the trans-splicing RNA compositions.

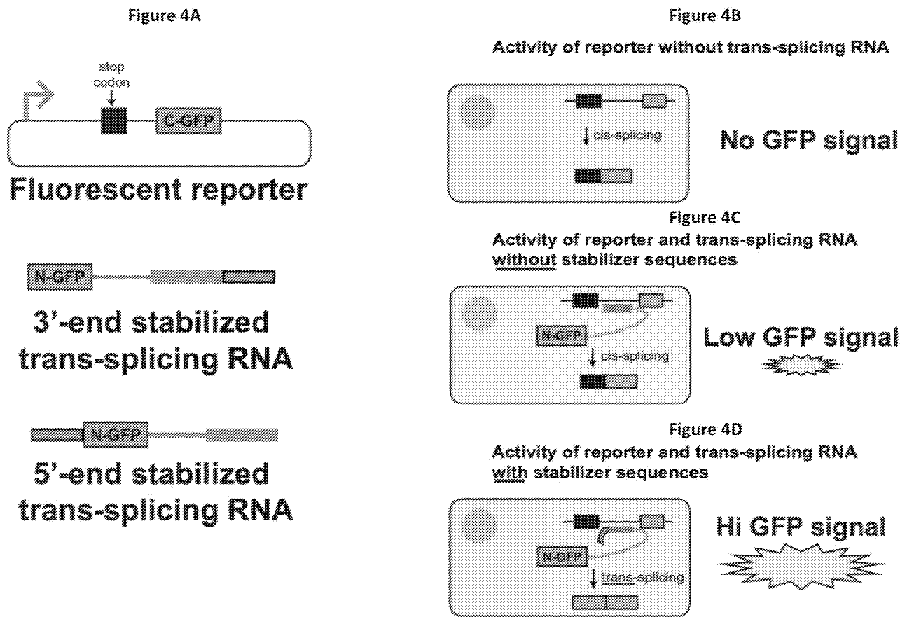

In one aspect, described herein is an RNA technology that enables replacement of arbitrary sequences within specific RNA molecules in living cells. In another aspect, described herein is a DNA technology that enable replacement of arbitration sequences within specific RNA molecules in living cells. In some embodiments, the DNA molecule encodes a gene or portion thereof to be transcribed. In some embodiments, the gene or portion thereof to be transcribed encodes an exonic sequence that corresponds to a missing or mutated portion of a target RNA. The technology, based on RNA trans-splicing, utilizes the naturally-existing spliceosome in human cells to provide the catalytic activity for this trans-splicing process. Without being limited by theory, RNA splicing occurs within RNA molecules where exons are concatenated and introns removed from immature messenger RNA molecules (pre-mRNAs) to form mature messenger RNA molecules (mRNAs). This process is referred to as cis-splicing. RNA trans-splicing is a process by which the spliceosome concatenates exons derived from distinct and separate RNA molecules. This process rarely occurs in human cells and state-of-the-art systems that promote RNA trans-splicing are active at low levels. The present disclosure provides for compositions that increase the efficiency of RNA trans-splicing. These improved RNA trans-splicing compositions can be used to replace mutated sequences within a target RNA molecule to address a human disease. Replacement of arbitrary RNA sequences is a general ability with innumerable specific applications a few of which have been explored as relevant demonstrations. RNA trans-splicing can insert engineered sequences into a target RNA to impart new activities to the target RNA such as altered RNA stability or altered RNA translation. This feature can be used to increase production of protein by a target RNA. In the broadest sense, this RNA trans-splicing technology can impart arbitrary changes to both coding and non-coding regions of target RNAs. In some instances, a trans-splicing molecule provided herein comprises stabilizing sequences. In some instances, a trans-splicing molecule provided herein does not comprise stabilizing sequences. In some instances, when trans-splicing sequences do not comprise stabilizing sequences, the exonic sequence to be trans-spliced into the target RNA is prone to degradation (FIG. 4B). In some instances, degradation of the trans-splicing sequences in the absence of stabilizing sequences is via cellular exonucleases. In some embodiments, as described in Example 1, stabilizing sequences that increase trans-splicing activity also increase the levels of trans-splicing molecule.

In some instances, stabilizing sequences are used to reduce degradation of generalized RNA molecules or trans-splicing molecules. A variety of RNA sequences derived from viruses, human and bacterial genes may block cellular exonuclease activity from the 3′ end (the exosome) or from the 5′ end (XRN1). In some instances, little is known of whether the presence of stabilizing sequences interfere with trans-splicing reactions and which putative stabilizing sequences function in the context of trans-splicing. As the activity of RNA stabilizing sequences may be context-dependent, in some instances a distinct group of stabilizing sequences that would function in the context of trans-splicing is described herein. In some instances, this is confirmed by experiments that indicate that activity of stabilizing sequences in other contexts may not be predictive of activity in trans-splicing.

Compositions comprising stabilizing sequences disclosed herein include any sequences that promote trans-splicing. Examples of stabilizing sequences include sequences derived or isolated from the following flaviviral genomes without limitation: Apoi virus, Aroa virus, Bagaza virus, Banzi virus, Bouboui virus, Bukalasa bat virus, Cacipacore virus, Carey Island virus, Cowbone Ridge virus, Dakar bat virus, Dengue virus, Edge Hill virus, Entebbe bat virus, Gadgets Gully virus, Ilheus virus, Israel turkey meningoencephalomyelitis virus, Japanese encephalitis virus, Jugra virus, Jutiapa virus, Kadam virus, Kedougou virus, Kokobera virus, Koutango virus, Kyasanur Forest disease virus, Langat virus, Louping ill virus, Meaban virus, Modoc virus, Montana myotis leukoencephalitis virus, Murray Valley encephalitis virus, Ntaya virus, Omsk hemorrhagic fever virus, Phnom Penh bat virus, Powassan virus, Rio Bravo virus, Royal Farm virus, Saboya virus, Saint Louis encephalitis virus, Sal Vieja virus, San Perlita virus, Saumarez Reef virus, Sepik virus, Tembusu virus, Tick-borne encephalitis virus, Tyuleniy virus, Uganda S virus, Usutu virus, Wesselsbron virus, West Nile virus, Yaounde virus, Yellow fever virus, Yokose virus, Zika virus,

Examples of stabilizing sequences also include sequences derived or isolated from the following long non-coding RNA genes without limitation: CDKN2B-AS1 [NR_003529]; BANCR [NR_047671]; CASC15 [NR_015410]; CRNDE [NR_034105]; EMX2OS [NR_002791]; EVF2 [NR_015448]; FENDRR [NR_036444]; FTX [NR_028379]; GAS5 [NR_002578]; HOTAIR [NR_003716]; HOTAIRM1 [NR_038366]; HOXA-AS3 [NR_038832]; HOXA11-AS [NR_002795]; JPX [NR_024582]; LHX5-AS1 [NR_126425]; LINC01578 [NR_037600]; LINC00261 [NR_001558]; MALAT1 [NR_002819.4]; MEG3 [NR_046473]; TUNAR [NR_038861]; MIAT [NR_033320]; NEAT1 [NR_028272]; NR2F1-AS1 [NR_021490]; LINC-PINT [NR_015431]; PSMA3-AS1 [NR_029434]; EMX2OS [ENSG00000229847]; PVT1 [NR_003367]; MEG8 [NR_024149]; RMST [NR_024037]; SENCR [NR_038908]; SIX3-AS1 [NR_103786]; SOX21-AS1 [NR_046514]; TERC [NR_001566]; TUG1 [NR_002323]; XIST [NR_001564], malat1 [NR_002847.3], Nfx1 [NM_023739.3], Ogt [NM_139144.4], Nlrp6 [NM_133946.2], Mixip1 [NM_021455.5], Leng8 [NM_001374609.1], Gcgr [NM_008101.2], Gck [NM_001287386.1], Acly [NM_001199296.1], Ccnl1 [NM_001355433.1], Ccnl2 [NM_207678.2], Chkb [NM_007692.6], LINC1609, MEG3, LINCRNA-P21, LXRBSV, SRA, BACE1AS, IPW, MEG3, AIR, KCNQ1OT1, RMST, SNHG5, KCNQ1OT1, LINC1610, ADAPT33, SNHG3, GAS5, NEAT1, NEAT2, BACE1AS, KCNQ1OT1, MALAT1, RIAN, SNHG1, SNHG4, SNHG5, SNHG6, ZFAS1, MENβ, and Sno.

Examples of trans-splicing stabilizing sequences also include sequences derived or isolated from the genomes of the following viruses without limitation: Kaposi's sarcoma-associated herpesvirus, turnip yellow mosaic virus, Plautia stali intestine virus.

Examples of trans-splicing stabilizing sequences also include sequences that form pseudoknots, triplexes, or other tertiary RNA structures. In some embodiments, the Stabilizing Domain forms a structure that blocks cellular nuclease activity. In some embodiments, the structure is a pseudoknot. In some embodiments, the structure is a stem-loop. In some embodiments, the Stabilizing Domain blocks directional cellular exonuclease activity. In some embodiments, the Stabilizing domain forms a triplex that blocks 3′-5′ exonuclease activity. In some embodiments, the Stabilizing domain forms an exonuclease-resistant RNA that blocks 5′-3′ exonuclease activity.

The presently disclosed RNA trans-splicing technology which involves the inclusion of specific stabilizing sequences for trans-splicing molecules is among the first to show RNA-trans-splicing with high efficiency against multiple RNA targets. Highly efficient RNA trans-splicing has at least three primary advantages over other RNA trans-splicing systems. First, this improved efficiency can replace defective RNA sequences at levels sufficient to reconstitute the activity of mutated genes to treat recessive genetic disorders. Indeed, treatment of many recessive gene disorders may require at least 30% efficiency, wherein 100% efficiency denotes complete replacement of a sequence within a Target RNA. Second, this improved efficiency can enable compositions as described herein to replace defective target RNA sequences at levels sufficient to treat dominant genetic disorders. For example, as a single mutated allele is sufficient to cause disease, many diseases in this class require highly-efficient replacement of mutated sequences as the mutated sequences may toxicity. As a result, even higher efficiency is required, e.g., at least about 70%. Thus, compositions as described herein can more effectively target broader classes of genetic disorders, i.e., even those with single mutated allele. Finally, the broad ability of RNA trans-splicing technology to modify multiple Target RNAs demonstrates the first broadly-applicable and efficient version of this technology. This is a very general capability, with this disclosure providing demonstrations of RNA trans-splicing system that can efficiently replace sequences with multiple target RNAs.

The inclusion of stabilizing sequences in trans-splicing molecules to form the present RNA trans-splicing technology is a general capability that may further allow the alteration of non-coding sequences within target RNAs. By replacing the 5′ or 3′ untranslated regions of Target RNAs with high efficiency, the methods and compositions described herein may allow the alteration of RNA behaviors such as translation or turnover. The net result of these effects is increased production of protein from Target RNAs or other downstream effects associated with altered RNA levels.

In some embodiments, the Stabilizing Domain sequence) are directly adjacent to the Antisense Domain. In some embodiments, the Stabilizing Domain sequence(s) are directly adjacent to the Replacement Domain.

In some embodiments, the Stabilizing Domain(s) are adjacent to the 5′ end of the trans-splicing molecule. In some embodiments, the Stabilizing Domain(s) are I nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, 50 nucleotides, 55 nucleotides, 60 nucleotides, 65 nucleotides, 70 nucleotides, 75 nucleotides, 80 nucleotides, 85 nucleotides, 90 nucleotides, 95 nucleotides, 100 nucleotides, 110 nucleotides, 120 nucleotides, 130 nucleotides, 140 nucleotides, 150 nucleotides, 160 nucleotides, 170 nucleotides, 180 nucleotides, 190 nucleotides, 200 nucleotides, 250 nucleotides, 300 nucleotides, 400 nucleotides, 500 nucleotides, more than 500 nucleotides, or any number of nucleotides in between distant from the 5′ end of the trans-splicing molecule.

In some embodiments, the Stabilizing Domain(s) are adjacent to the 3′ end of the trans-splicing molecule. In some embodiments, the Stabilizing Domain(s) are 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, 50 nucleotides, 55 nucleotides, 60 nucleotides, 65 nucleotides, 70 nucleotides, 75 nucleotides, 80 nucleotides, 85 nucleotides, 90 nucleotides, 95 nucleotides, 100 nucleotides, 110 nucleotides, 120 nucleotides, 130 nucleotides, 140 nucleotides, 150 nucleotides, 160 nucleotides, 170 nucleotides, 180 nucleotides, 190 nucleotides, 200 nucleotides, 250 nucleotides, 300 nucleotides, 400 nucleotides, 500 nucleotides, more than 500 nucleotides, or any number of nucleotides in between distant from the 3′ end of the trans-splicing molecule.

In some embodiments, the Stabilizing Domain(s) are 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, 50 nucleotides, 55 nucleotides, 60 nucleotides, 65 nucleotides, 70 nucleotides, 75 nucleotides, 80 nucleotides, 85 nucleotides, 90 nucleotides, 95 nucleotides, 100 nucleotides, 110 nucleotides, 120 nucleotides, 130 nucleotides, 140 nucleotides, 150 nucleotides, 160 nucleotides, 170 nucleotides, 180 nucleotides, 190 nucleotides, 200 nucleotides, 250 nucleotides, 300 nucleotides, 400 nucleotides, 500 nucleotides, more than 500 nucleotides, or any number of nucleotides in between distant from the first nucleotide of the Replacement Domain or Antisense Domain in the 5′ direction.

In some embodiments, Stabilizing Domain(s) are 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, 50 nucleotides, 55 nucleotides, 60 nucleotides, 65 nucleotides, 70 nucleotides, 75 nucleotides, 80 nucleotides, 85 nucleotides, 90 nucleotides, 95 nucleotides, 100 nucleotides, 110 nucleotides, 120 nucleotides, 130 nucleotides, 140 nucleotides, 150 nucleotides, 160 nucleotides, 170 nucleotides, 180 nucleotides, 190 nucleotides, 200 nucleotides, 250 nucleotides, 300 nucleotides, 400 nucleotides, 500 nucleotides, more than 500 nucleotides, or any number of nucleotides in between distant from the last nucleotide of the Replacement Domain or Antisense Domain in the 3′ direction.

In some embodiments, the stabilized RNA trans-splicing molecule comprises one or more Stabilizing Domains. In some embodiments, the Intronic Domain comprises 2 or more Stabilizing Domains. In some embodiments, the stabilized RNA trans-splicing molecule comprises 3, 4, 5, 6, 7, 8, 9, 10, 11, 12, 13, 14, 15, 20, 30, 40, 50, 75, 100, 200, 300 or more Stabilizing Domains.

In some embodiments, the trans-splicing nucleic acid is RNA, DNA, a DNA/RNA hybrid, and/or comprises at least one of a nucleic acid analog, a chemically-modified nucleic acid, or a chimera composed of two or more nucleic acids or nucleic acid analogs. As used herein, the term “nucleic acid analog” refers to a compound having structural similarity to a canonical purine or pyrimidine base occurring in DNA or RNA. The nucleic acid analog may contain a modified sugar and/or a modified nucleobase, as compared to a purine or pyrimidine base occurring naturally in DNA or RNA. In some embodiments, the nucleic acid analog is a 2′-deoxyribonucleoside, 2′-ribonucleoside, 2′-deoxyribonucleotide or a 2′-ribonucleotide, wherein the nucleobase includes a modified base (such as, for example, xanthine, uridine, oxanine (oxanosine), 7-methlguanosine, dihydrouridine, $-methylcytidine, C3 spacer, 5-methyl dC, 5˜ hydroxybutynl-2′-deoxyuridine, 5-nitroindole, 5-methyl iso-deoxycytosine, iso deoxyguanosine, deoxyuradine, iso deoxycytidine, other 0-1 purine analogs, N-6-hydroxylaminopurine, nebularine, 7-deaza hypoxanthine, other 7-deazapurines, and 2-methyl purines). In some embodiments, the nucleic acid analog may be selected from the group consisting of inosine, 7-deaza-2′-deoxyinosine, 2′-aza-2′-deoxyinosine, PNA-inosine, morpholino-inosine, LNA-inosine, phosphoramidate-inosine, 2′-O-methoxyethyl-inosine, and 2′-OMe-inosine. In other embodiments the nucleic acid analog is a nucleic acid mimic (such as, for example, artificial nucleic acids and xeno nucleic acids (XNA).

### Identification of Stabilizing Sequences for Trans-Splicing Molecules

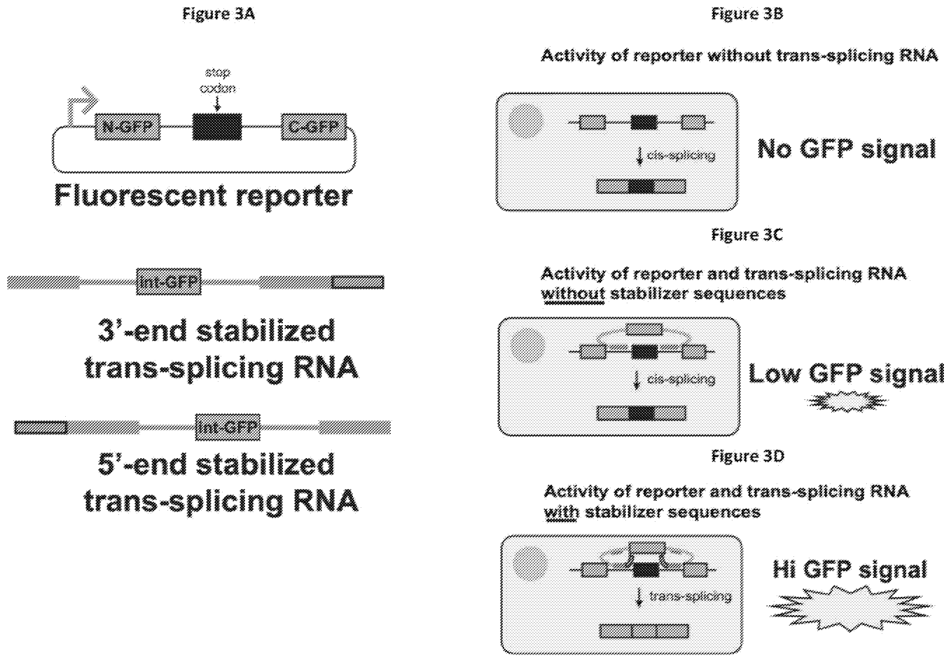

The present disclosure provides compositions comprising a trans-splicing nucleic acid with one or more stabilizing sequences. The stabilizing sequences described herein may increase the efficiency of nucleic acids at replacing sequences in a target RNA. For example, stabilizing sequences described herein may increase the efficiency of RNA-trans-splicing when placed at the 5′ and/or 3′ end of an RNA in a model trans-splicing molecule. The trans-splicing molecule may comprise, e.g., DNA or RNA. The trans-splicing RNA may be transcribed from a DNA molecule comprising Stabilizing Domains. In some embodiments, the DNA molecule comprises a Replacement Domain. In some embodiments, the Replacement Domain is transcribed into an RNA sequence, such as an RNA sequence that corresponds to a missing or mutated portion of a target RNA sequence. In some embodiments, the DNA or RNA trans-splicing molecule comprises an Antisense Domain. In some embodiments, the Antisense Domain of the DNA molecule is transcribed into an Antisense Domain comprising RNA. In some embodiments, the Antisense Domain comprising RNA is complementary to the target RNA or a portion thereof. In some embodiments, the Antisense Domain binds to the target RNA. In some embodiments, the antisense RNA is chosen so that successful trans-splicing causes removal of micro-open reading frames in the target RNA. In some embodiments, the trans-splicing DNA or RNA molecule comprises an Intronic Domain. The Intronic Domain of the DNA molecule can be transcribed into an Intronic Domain comprising RNA. In some embodiments, the Intronic Domain promotes the trans-splicing reaction between a trans-splicing RNA molecule and the target RNA. In some embodiments, the Intronic Domains carry binding sites that are preferentially-targeted by RNA-binding proteins with disease-causing mutations. In some embodiments, the trans-splicing DNA or RNA molecule comprises a Stabilizing Domain. In some embodiments, the trans-splicing DNA or RNA molecule comprises one or more Stabilizing Domains. In some embodiments, the DNA molecule comprising one or more Stabilizing Domains encodes an RNA molecule comprising the one or more Stabilizing Domains. In some embodiments, the DNA molecule comprising one or more Stabilizing Domains can be transcribed into an RNA molecule comprising the one or more Stabilizing Domains. In some embodiments, the Stabilizing Domain carries sequences that block the activity of cellular nucleases. In some instances, blocking the activity of cellular nucleases may increase the effective level of the trans-splicing molecule in human cells. These variant trans-splicing molecules target a split GFP reporter assay that fluoresces only after successful activity of the RNA trans-spicing molecule (FIGS. 3-5). This assay is qualitative, not fully quantitative, but is useful because it is what end-users in cell biology often use when attempting to answer scientific questions about the presence, absence, or general magnitude of a transcript. GFP trans-splicing reporters has, accordingly, been widely used in the study of RNA trans-splicing technologies. A GFP reporter similar to a published system (3) was used to compare the relative influence of different stabilizing sequences on the efficiency of the trans-splicing reaction.

FIGS. 3-5 describe a schematic of the plasmids used in the trans-splicing activity assays. Experiments were conducted with either a transiently-transfected reporter and trans-splicing molecule or systems packaged in lentivirus. The sequences described herein includes sequences that may block the activity of cellular nucleases, stabilize trans-splicing molecules, and enhance transplicing, Not all sequences that block the activity of cellular nucleases stabilize trans-splicing molecules and enhance trans-splicing. As used herein, these trans-splicing-specific stabilizing sequences are termed “stabilizer sequences”.

### Use of Stabilizing Sequences to Increase the Translation of Specific Target RNAs

Compositions as described herein may modulate the level of protein produced. In addition to replacing specific mutated sequences within RNAs with non-mutated sequences, another useful operation of compositions as described herein on target mRNA molecules is increasing the protein produced. There have been many attempts to address this problem of insufficient protein production from specific mRNAs but each approach has major shortcomings. Indeed, small molecule drugs that increase translation by promoting stop codon read-through suffer extensive off-targets. For example, such small molecule drugs may promote read-through on non-target mRNA. Further, pre-mature stop codons are one of many causes of insufficient protein levels. Other technologies, e.g., engineered tRNAs to block pre-mature termination codons, also suffer from this same fundamental issue. An RNA trans-splicing system, by contrast, can replace sequences in any target mRNA with translation-amplifying sequences to increase protein production. Furthermore, compositions as described herein may have greater target specificity to effect therapy to the appropriate target RNA. Described herein are methods of efficient RNA trans-splicing mediated by stabilizing sequences can address this long-felt but unmet need of a method to promote targeted amplification of protein production from specific mRNAs.

Compositions as described herein can treat mutated target RNA, and thereby amplify protein production form the target RNA. For example, Myotonic dystrophy is caused by RNAs that carry repetitive ‘CUG’ tracts that bind the splicing factor MBNL1. Titration of MBNL1 away from its typical targets causes widespread dysfunction of RNA alternative splicing and is responsible for most manifestations of disease in patients. Described herein are methods of increasing MBNL1 protein production with an efficient RNA trans-splicing approach can address this disease via production of sufficient MBNL1 protein to reconstitute its typical activities in alternative splicing regulation.

Described herein is an RNA trans-splicing system carrying various stabilizing sequences such as, a Woodchuck Hepatitis Virus (WHV) post-transcriptional Regulatory Element (WPRE) to assess the ability of an RNA trans-splicing system containing stabilizing sequences to increase protein production from specific mRNAs. Also described herein is a reporter that contains a firefly luciferase coding sequence and the last 2 exons and intervening intron of MBNL1. This assay is qualitative, not fully quantitative, but is useful because it is what end-users in cell biology often use when attempting to answer scientific questions about the presence, absence, or general magnitude of a transcript. Indeed, this reporter is based on the pMIR-GLO luciferase vector that is used to assess the stability and protein production from a model mRNA.

Experiments were conducted with either transiently-transfected reporter and trans-splicing molecule or systems packaged in lentivirus.

Stabilizing Sequences that Protect the 5′ End of Trans-Splicing RNAs

The present disclosure provides a nucleic acid molecule comprising one or more stabilizing sequences to prevent or attenuate degradation of the nucleic acid molecule. In some embodiments, a DNA or RNA molecule provided herein comprises one or more stabilizing sequences to prevent degradation of the nucleic acid molecule. The DNA molecule comprising one or more stabilizing sequences may encode an RNA molecule comprising one or more stabilizing sequences. The DNA molecule comprising one or more stabilizing sequences may be transcribed into an RNA molecule comprising one or more stabilizing sequences. The degradation may be caused by, e.g., the activity of exonucleases. The exonuclease may act in the 5′ to 3′ direction on RNA.

In some embodiments, the stabilizing sequence comprises RNA. In some embodiments, the stabilizing sequence comprises DNA. In some embodiments, the stabilizing sequence comprising DNA encodes a stabilizing sequence comprising RNA. In some embodiments, the DNA molecule is transcribed into a messenger RNA

In some embodiments of the compositions of the present disclosure, the stabilizing sequence is derived from a flavivirus. In some embodiments, the Stabilizing Domain is an exonuclease-resistant RNA (“xrRNA”) that block 5′-3′ exonuclease activity and is derived or isolated from a viral genome selected from the group consisting of: Turnip yellow mosaic virus, Apoi virus, Aroa virus, Bagaza virus, Banzi virus, Bouboui virus, Bukalasa bat virus, Cacipacore virus, Carey Island virus, Dakar bat virus, Cowbone Ridge virus, Dengue virus, Edge Hill virus, Entebbe bat virus, Gadgets Gully virus, Ilheus virus, Israel turkey meningoencephalomyelitis virus, Japanese encephalitis virus, Jugra virus, Jutiapa virus, Kadam virus, Kedougou virus, Kokobera virus, Koutango virus, Kyasanur Forest disease virus, Langat virus, Louping ill virus, Meaban virus, Modoc virus, Montana myotis leukoencephalitis virus, Murray Valley encephalitis virus, Ntaya virus, Omsk hemorrhagic fever virus, Phnom Penh bat virus, Powassan virus, Rio Bravo virus, Royal Farm virus, Saboya virus, Saint Louis encephalitis virus, Sal Vieja virus, San Perlita virus, Saumarez Reef virus, Sepik virus, Tembusu virus, Tick-borne encephalitis virus, Tyuleniy virus, Uganda S virus, Wesselsbron virus, Usutu virus, West Nile virus, Yaounde virus, Yellow fever virus, Yokose virus, Zika virus.

In some embodiments, the stabilizing sequence that protects the trans-splicing molecule from exonucleases that act in the 5′ to 3′ direction comprises or consists of sequences from Kunjun virus. The sequence may be a DNA sequence. The sequence may be an RNA sequence. In some embodiments, the sequences from Kunjin virus comprise or consist of: TTAGTGAGGATGTCAGACCACGGCCATGGCGTGCCACTCTGCGGAGAGTGCAGTCT GCGACAGTGCCCCAGGAGGACTGGG (SEQ ID NO: 1). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 1. In some embodiments, the stabilizing sequence comprises a sequence encoded by SEQ ID NO. 1. The stabilizing sequence may be transcribed into an RNA molecule.

In some embodiments, the stabilizing sequence that protects the trans-splicing molecule from exonucleases that act in the 5′ to 3′ direction comprises or consists of sequences from Cell fusing agent virus. The sequence may be a DNA sequence. The sequence may be an RNA sequence. In some embodiments, the sequences from Cell fusing agent virus comprise or consist of: ACAGGAGCAGGGCATGAAAATGTCGGGCATGACGAACCCGCTCCCCCGAGTCCCCT GGCAACAGGGT (SEQ ID NO: 2). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 2. In some embodiments, the stabilizing sequence comprises a sequence encoded by SEQ ID NO. 2. The stabilizing sequence may be transcribed into an RNA molecule.

In some embodiments, the stabilizing sequence that protects the trans-splicing molecule from exonucleases that act in the 5′ to 3′ direction comprises or consists of sequences from Flavivirus Tick-borne encephalitis virus. The sequence may be a DNA sequence. The sequence may be an RNA sequence. In some embodiments, the sequences from Flavivirus Tick-borne encephalitis virus comprise or consist of: CACAGATCATGGAATGATOCGGCAGCGCGCGAGAGCAACGGGGAAGTGGTGGCAC CCGACGCACCATCCATGAAGCAATACTTCGTGAGACCCCCCCTGACCAGCAAAGGG GGCAGACCGGTCAGGGGTGAGGAATGCCCCCAGAGTGCATTACGGCAGCACGCCAG TGAGAGTGGCGACGGGAAAATGGTCGATCCCGACGTAGGGCACTCTGAAAAATTTT GTGAGACC (SEQ ID NO: 3). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 3. In some embodiments, the stabilizing sequence comprises a sequence encoded by SEQ ID NO. 3. The stabilizing sequence may be transcribed into an RNA molecule.

In some embodiments, the stabilizing sequence that protects the trans-splicing molecule from exonucleases that act in the 5′ to 3′ direction comprises or consists of sequences from Murine leukemia virus. The sequence may be a DNA sequence. The sequence may be an RNA sequence. In some embodiments, the sequences from Murine leukemia virus comprise or consist of: TGGAAAAAATGCGAGTGAGGGCAACTCTGGGATTAGCTCAATGGGTGTGACGACCC TACCCTTCCGCATTTGTAAATAATTGAGCCAGTCATTTCCGTAGGGAAGAGAGTTAT TCGCTCCTCTCGAGATTGAGCGGCCTGCTCCTTGGAGCATGAGATGGGAGGCCCGAA (SEQ ID NO 4). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 4. In some embodiments, the stabilizing sequence comprises a sequence encoded by SEQ ID NO. 4. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number AF346759.1. The sequence may be a DNA sequence. The sequence may be an RNA sequence. In some embodiments, the sequences from this flavivirus with genome accession number AF346759.1 comprise or consist of: GAAAGGCAAGGTACGGATTAGCCGTAGGGGCTTGAGAACCCCCCCTCCCCACTCAT TTTATTTCCTCTATGAGGAAGG (SEQ ID NO: 5). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 5. In some embodiments, the stabilizing sequence comprises a sequence encoded by SEQ ID NO. 5. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number AF346759.1. The sequence may be a DNA sequence. The sequence may be an RNA sequence. In some embodiments, the sequences from this flavivirus with genome accession number AF346759.1 comprise or consist of; TTTGGGCAAGGTGCAGGTTAGCTGCAGGGGCTTGAAAAACCCCCCCCCCCATTCAA GACTTTTAGTGCATTAGTT (SEQ ID NO:6). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 6. In some embodiments, the stabilizing sequence comprises a sequence encoded by SEQ ID NO. 6. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_008604.2. The sequence may be a DNA sequence. The sequence may be an RNA sequence. In some embodiments, the sequences from this flavivirus with genome accession number NC_008604.2 comprise or consist of: ACCCCGTAAGGAAGGACAAGGCTGTCCTTGACTACTAACGACACTCCGGCCCCAGT TCCCAGAGCCAGGGTTTTAGCTCC (SEQ ID NO: 7). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 7. In some embodiments, the stabilizing sequence comprises a sequence encoded by SEQ ID NO. 7. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_008604.2. The sequence may be a DNA sequence. The sequence may be an RNA sequence. In some embodiments, the sequences from this flavivirus with genome accession number NC_008604.2 comprise or consist of: CGCGCGCAAGGAAGGACATGGCTGTCCTTGGGTACGAACGACACCCCGCCCCCAGT TCTCAAGGTTAGAGTTATAACCTC (SEQ ID NO: 8). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 8. In some embodiments, the stabilizing sequence comprises a sequence encoded by SEQ ID NO. 8. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_008604.2. The sequence may be a DNA sequence. The sequence may be an RNA sequence. In some embodiments, the sequences from this flavivirus with genome accession number NC_008604.2 comprise or consist of: CCATCGCAAGGGAGGATTTTCCTCGGGTACTGACCATACCCCGACCCCAGTCCGATA GGTCATGGAATGACCCC (SEQ ID NO: 9). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 9. In some embodiments, the stabilizing sequence comprises a sequence encoded by SEQ ID NO. 9. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_008604.2. The sequence may be a DNA sequence. The sequence may be an RNA sequence. In some embodiments, the sequences from this flavivirus with genome accession number NC_008604.2 comprise or consist of: CTCCCGTAAGGAAAGCGCAAGCTTTGAGCATTGACAACGCTCCGGCCCCAGTCCCCC AGGTTATGGGAGAATAACCC (SEQ ID NO: 10). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 10. In some embodiments, the stabilizing sequence comprises a sequence encoded by SEQ ID NO. 10. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number HE574574.1. The sequence may be a DNA sequence. The sequence may be an RNA sequence. In some embodiments, the sequences from this flavivirus with genome accession number HE574574.1 comprise or consist of: CTAGCGCAAGGAAGGAAAGTCGCAGACTACCTTGGGTGTTGACGACACTCCGCCCC CAGTCACCTTGGCCGAAGGTTAAACGGCAT (SEQ ID NO: 11). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 11. In some embodiments, the stabilizing sequence comprises a sequence encoded by SEQ ID NO. 11. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number HE574574.1. The sequence may be a DNA sequence. The stabilizing sequence may be an RNA sequence. In some embodiments, the sequences from this flavivirus with genome accession number HE574574.1 comprise or consist of: CCACGCAATGGGAGGCATCATTTCGCCTCCGGGTGCTGACTACTCCCCGTCCCCGTC CCTAGGTCAAGTGAATGACCCCGTGATGTTGTGATGACATCATACCAGGCTTOGCAT CCTGGCAACTGCCACCCGCAAGGGGAGGGTTTTCTAACTCTCCGGGTGTTGACGACA CCCCGGCCCCAGTCCCCAAGGTCTTGGGAAAAAGACCCCGAAGTGTTGCAAGGACA CTAATCACCGAAAGGTGAGGGCGCACAGGATC (SEQ ID NO: 12). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 12. In some embodiments, the stabilizing sequence comprises a sequence encoded by SEQ ID NO. 12. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number HE574574.1. The sequence may be a DNA sequence. The sequence may be an RNA sequence. In some embodiments, the sequences from this flavivirus with genome accession number HE574574.1 comprise or consist of: CACCCGCAAGGGGAGGGTTTTCTAACTCTCCGGGTGTTGACGACACCCCGGCCCCAG TCCCCAAGGTCTTGGGAAAAAGACCCC (SEQ ID NO: 13). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 13. In some embodiments, the stabilizing sequence comprises a sequence encoded by SEQ ID NO. 13. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_012671.1. The sequence may be a DNA sequence. The sequence may be an RNA sequence. In some embodiments, the sequences from this flavivirus with genome accession number NC_012671.1 comprise or consist of: TCAGCGCAAGGAAGGGAAGCTTGAGGCTACCTTAGGTGGTGACGACACCTCGCCCC CAGTCTCCCGGGTTGGGGATAATACAACCTC (SEQ ID NO: 14). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 14. In some embodiments, the stabilizing sequence comprises a sequence encoded by SEQ ID NO. 14. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_012671.1. The sequence may be a DNA sequence. The sequence may be an RNA sequence. In some embodiments, the sequences from this flavivirus with genome accession number NC_012671.1 comprise or consist of: CCCCCGCAAGGAGGGGTGTGTGTTTCACGCCCCTGGGAGTTAACGATTCTCCGGCCC CAGTTCCTAGGTCCAGGGAGGGCCCC (SEQ ID NO: 15). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 15. In some embodiments, the stabilizing sequence comprises a sequence encoded by SEQ ID NO. 15. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_012671.1. The sequence may be a DNA sequence. The sequence may be an RNA sequence. In some embodiments, the sequences from this flavivirus with genome accession number NC_012671.1 comprise or consist of: CCCGCGCAAGGAAGGACACGTAAATCACGTGTTCTTGGGAGTTGACGACTCTCCGC CCCCAGTCCCCAGGTCAGGGTATGACTCC (SEQ ID NO: 16). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 16. In some embodiments, the stabilizing sequence comprises a sequence encoded by SEQ ID NO. 16. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_012671.1. The sequence may be a DNA sequence. The sequence may be an RNA sequence. In some embodiments, the sequences from this flavivirus with genome accession number NC_012671.1 comprise or consist of: CCACCGCAAGGGAGAGGGATCCCCTCTCGGGTTTGGACGACACCCCGGCCCCAGTC CCCCAGGTCATGGGAAAAACTGACCCC (SEQ ID NO: 17). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 17. In some embodiments, the stabilizing sequence comprises a sequence encoded by SEQ ID NO. 17. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_021069.1. The sequence may be a DNA sequence. The sequence may be an RNA sequence. In some embodiments, the sequences from this flavivirus with genome accession number NC_021069.1 comprise or consist of: TCAGCGCAAGGAAGGAAAGCTGGACGCTACCTTAGGTGGTGACGACACCTOGCCCC CAGTCTCCCAGGTTGGGGATCGTACAACTTC (SEQ ID NO: 18). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 18. In some embodiments, the stabilizing sequence comprises a sequence encoded by SEQ ID NO. 18. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_021069.1. The sequence may be a DNA sequence. The sequence may be an RNA sequence. In some embodiments, the sequences from this flavivirus with genome accession number NC_021069.1 comprise or consist of: CCCCCGCAAGGAGGGACGTGTGCATCACGTTTCTGGGAGTTAACGGCTCTCCGGCCC CAGTTCCTAGGTCCAGGTAGGATCCC (SEQ ID NO: 19). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 19. In some embodiments, the stabilizing sequence comprises a sequence encoded by SEQ ID NO. 19. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_021069.1. The sequence may be a DNA sequence. The sequence may be an RNA sequence. In some embodiments, the sequences from this flavivirus with genome accession number NC_021069.1 comprise or consist of: CCCGCGCAAGGAAGGATGCGATGAAAACGCTTGTCCTTGGGAGTTGACGACTCTCC GCCCCCAGTCCCCAGGTCAGGGTATGACCCC (SEQ ID NO: 20). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 20. In some embodiments, the stabilizing sequence comprises a sequence encoded by SEQ ID NO. 20. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_021069.1. The sequence may be a DNA sequence. The sequence may be an RNA sequence. In some embodiments, the sequences from this flavivirus with genome accession number NC_021069.1 comprise or consist of: CCACCGCAAGGGAGAGGGATTCCCTCTCGGGTGTGGACGACACCCCGGCCCCAGTC CCCTAGGTCATGGGAAAAACTGACCCC (SEQ ID NO: 21). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 21. In some embodiments, the stabilizing sequence comprises a sequence encoded by SEQ ID NO. 21. The stabilizing sequence may be transcribed into an RNA molecule,

In some embodiments, the stabilizing sequence that protects the trans-splicing molecule from exonucleases that act in the 5′ to 3′ direction comprises or consists of sequences from a flavivirus with genome accession number KX652378.1. The sequence may be a DNA sequence. The sequence may be an RNA sequence. In some embodiments, the sequences from this flavivirus with genome accession number KX652378.1 comprise or consist of: CTAGCGCAAGGAAGGAAAGTCGCAGACTACCTTGGGTGTTGACGACACTCCGCCCC CAGTCACCTTGGCCAAAGGTTAAATGGCAT (SEQ ID NO: 22). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 22. In some embodiments, the stabilizing sequence comprises a sequence encoded by SEQ ID NO. 22. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number KX652378.1. The sequence may be a DNA sequence. The sequence may be an RNA sequence. In some embodiments, the sequences from this flavivirus with genome accession number KX652378.1 comprise or consist of: CCCACGCAAGGGGAGGCATCATATTGCCTCCGGGTGCTGACGACACCCCGTCCCCA GTCCCTAGGTCAAGTGAATGACCCC (SEQ ID NO: 23). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 23. In some embodiments, the stabilizing sequence comprises a sequence encoded by SEQ ID NO. 23. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number KX652378.1. The sequence may be a DNA sequence. The sequence may be an RNA sequence. In some embodiments, the sequences from this flavivirus with genome accession number KX652378.1 comprise or consist of: CACCCGCAAGGGGGGAGTTTTCTAACTCCCCGGGTGTTGACGACACCCCGGCCCCA GTCCCCAAGGTCTTGGGAAAAAGACCCC (SEQ ID NO: 24). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 24. In some embodiments, the stabilizing sequence comprises a sequence encoded by SEQ ID NO. 24. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_001564.2. The sequence may be a DNA sequence. The sequence may be an RNA sequence. In some embodiments, the sequences from this flavivirus with genome accession number NC_001564.2 comprise or consist of: ACAGGAGCAGGGCATGAAAATGTCGGGCATGACGAACCCGCTCCCCCGAGTCCCCT GGCAACAGGGTGTGTTCC (SEQ ID NO: 25). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 25. In some embodiments, the stabilizing sequence comprises a sequence encoded by SEQ ID NO. 25. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_001564.2. The sequence may be a DNA sequence. The sequence may be an RNA sequence. In some embodiments, the sequences from this flavivirus with genome accession number NC_001564.2 comprise or consist of: TTCGAGCAGGGCACATTAGTGTCOGGCGTGACGCACCCGCTCCCCTCAGTCCCCTGT GCAACAGGGAGGGCACTT (SEQ ID NO: 26). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 26. In some embodiments, the stabilizing sequence comprises a sequence encoded by SEQ ID NO. 26. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_001564.2. The sequence may be a DNA sequence. The sequence may be an RNA sequence. In some embodiments, the sequences from this flavivirus with genome accession number NC_001564.2 comprise or consist of: ACGGGGCAACAGGGAGAAATCCCGGGGTAGCGAACCTCCTCCGTTAATGTGAAAAA GTATGGGGAAAGAACTCATCT (SEQ ID NO: 27). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 27. In some embodiments, the stabilizing sequence comprises a sequence encoded by SEQ ID NO. 27. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_012932.1. The sequence may be a DNA sequence. The sequence may be an RNA sequence. In some embodiments, the sequences from this flavivirus with genome accession number NC_012932.1 comprise or consist of: TCAGAGCAGGGCACAATAGTGTCOGGCCTGACGACCCCGCTCCCCCGAGTCGOCCA ACGGAGTTTGGCTCAACTCTAA (SEQ ID NO: 28). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 28. In some embodiments, the stabilizing sequence comprises a sequence encoded by SEQ ID NO. 28. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_012932.1. The sequence may be a DNA sequence. The sequence may be an RNA sequence. In some embodiments, the sequences from this flavivirus with genome accession number NC_012932.1 comprise or consist of: GCAGCGCAGGGCATGAAAATGTCGGGCCTGACGAACCCGCOCACCCGAGTCCCCCA GTTGGGGAAGGGATCCTTGCAT (SEQ ID NO: 29). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 29. In some embodiments, the stabilizing sequence comprises a sequence encoded by SEQ ID NO. 29. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_012932.1. The sequence may be a DNA sequence. The sequence may be an RNA sequence. In some embodiments, the sequences from this flavivirus with genome accession number NC_012932.1 comprise or consist of: GCGGAGCAACAGGGAGAAATCCCGGGGAATTGCGAACCCCCTCCGAAATGTGAAA AATTATGGGGAAAAGTACCCATCT (SEQ ID NO: 30). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 30. In some embodiments, the stabilizing sequence comprises a sequence encoded by SEQ ID NO. 30. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number KJ741266.1. The sequence may be a DNA sequence. The sequence may be an RNA sequence. In some embodiments, the sequences from this flavivirus with genome accession number KJ741266.1 comprise or consist of: GAGCAGGGCACAACAGTGTCGGGCCTGACGACCCCGCTCCCCCGAGTCACCAACTG GAGTTTGGCTCAACTCCAA (SEQ ID NO: 31). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 31. In some embodiments, the stabilizing sequence comprises a sequence encoded by SEQ ID NO. 31. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_031327.1. The sequence may be a DNA sequence. The sequence may be an RNA sequence. In some embodiments, the sequences from this flavivirus with genome accession number NC_031327.1 comprise or consist of: GGAAGGCAACACACCAGTAATCTGGTGGGGTGAGTTGCGACACCCCCTTGTGAGCA ACCGACCTAGCCGGTCTATTGACCGGCTTG (SEQ ID NO: 32). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 32. In some embodiments, the stabilizing sequence comprises a sequence encoded by SEQ ID NO. 32. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_024299.2. The sequence may be a DNA sequence. The sequence may be an RNA sequence. In some embodiments, the sequences from this flavivirus with genome accession number NC_024299.2 comprise or consist of: ACAGGGCAACAGGGATCGCCAACATCAGATCCCGGGTGAGTGACGACACCCCCCAT GTGAATCGTCAACTTAGGAACACATTCAAATAGAGGA (SEQ ID NO: 33). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 33. In some embodiments, the stabilizing sequence comprises a sequence encoded by SEQ ID NO. 33. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_024299.2. The sequence may be a DNA sequence. The sequence may be an RNA sequence. In some embodiments, the sequences from this flavivirus with genome accession number NC_024299.2 comprise or consist of: ACAGGGCAACAGGGATCACCAACATCAGATCCCGGGTGAGTGACGACACCCCCCAT GTGAATCGTCGATACAAAAACACGATAGG (SEQ ID NO: 34). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 34. In some embodiments, the stabilizing sequence comprises a sequence encoded by SEQ ID NO. 34. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_024299.2. The sequence may be a DNA sequence. The sequence may be an RNA sequence. In some embodiments, the sequences from this flavivirus with genome accession number NC_024299.2 comprise or consist of: ACACCGCAAGGAAGAGAAATCTTGGGTGGTAACAACACCCCGGCCCCAGTTCTCGC GTOCCACGAGTCATTGGCACAA (SEQ ID NO: 35). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 35. In some embodiments, the stabilizing sequence comprises a sequence encoded by SEQ ID NO. 35. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_024299.2. The sequence may be a DNA sequence. The sequence may be an RNA sequence. In some embodiments, the sequences from this flavivirus with genome accession number NC_024299.2 comprise or consist of: AAACCGCAAGGAAGGAGCAATCCTTGGGTATTAACGACACCCCGGCCCCAGTTCCC GAAGTCAAGGGGACCCTTGACCC (SEQ ID NO: 36). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 36. In some embodiments, the stabilizing sequence comprises a sequence encoded by SEQ ID NO. 36. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_034242.1. The sequence may be a DNA sequence. The sequence may be an RNA sequence. In some embodiments, the sequences from this flavivirus with genome accession number NC_034242.1 comprise or consist of: CATTGCAGCAGAGATTTATCTCGGGGGAGTTACGCCCCTCCATTGCCAGTAGAGTTT GCATGTCTCTATAAACATGACGTT (SEQ ID NO: 37). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 37. In some embodiments, the stabilizing sequence comprises a sequence encoded by SEQ ID NO. 37. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_027817.1. The sequence may be a DNA sequence. The sequence may be an RNA sequence. In some embodiments, the sequences from this flavivirus with genome accession number NC_027817.1 comprise or consist of: CCATTGCCAGTAGAGTTTGCATGTCTCTATAAACATGACGTTCTGACTGACTA (SEQ ID NO: 102). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 102. In some embodiments, the stabilizing sequence comprises a sequence encoded by SEQ ID NO, 102. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_027817.1. The sequence may be a DNA sequence. The sequence may be an RNA sequence. In some embodiments, the sequences from this flavivirus with genome accession number NC_027817.1 comprise or consist of: AATATGGCAGCAGAGCTTGTCTCGGGGATTCACGCTCCCCCCATTGTGAGTGTGTCG AACTGGTTTCGAAGGACGTCTAGAA (SEQ ID NO: 38). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 38. In some embodiments, the stabilizing sequence comprises a sequence encoded by SEQ ID NO. 38. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_027817.1. The sequence may be a DNA sequence. The sequence may be an RNA sequence. In some embodiments, the sequences from this flavivirus with genome accession number NC_027817.1 comprise or consist of: ATCCGGCAACAGAGAGTTTATGTCTCGGGGCCTCACGCACCCCCCGTTGTGAGTGAA GTCCTTTCTGGCCATTTAGTGGTCAGGAAGGG (SEQ ID NO: 39). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 39. In some embodiments, the stabilizing sequence comprises a sequence encoded by SEQ ID NO. 39. The stabilizing sequence may be transcribed into an RNA molecule.

In some embodiments, the stabilizing sequence that protects the trans-splicing molecule from exonucleases that act in the 5° to 3′ direction comprises or consists of sequences from a flavivirus with genome accession number NC_027817.1. The sequence may be a DNA sequence. The sequence may be an RNA sequence. In some embodiments, the sequences from this flavivirus with genome accession number NC_027817.1 comprise or consist of: GCAGGGCAACAAAGTTCTAACGAACTAGGGTGAGTAGCGTCACCCCCCGGTTGTGA AAACGATTGCGACTAGAACTAAAGTCGAGAGTCTC (SEQ ID NO: 40). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 40. In some embodiments, the stabilizing sequence comprises a sequence encoded by SEQ ID NO. 40. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_005064.1. The sequence may be a DNA sequence. The sequence may be an RNA sequence. In some embodiments, the sequences from this flavivirus with genome accession number NC_005064.1 comprise or consist of: TTAGAGCAGGGCACGAAAGTGTCGGGCATGACGCACCCGCTCCCCCGAGTCCCCTG AAAATAGGGTGGGCAATGCACTCCT (SEQ ID NO: 41). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 41. In some embodiments, the stabilizing sequence comprises a sequence encoded by SEQ ID NO. 41. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_005064.1. The sequence may be a DNA sequence. The sequence may be an RNA sequence. In some embodiments, the sequences from this flavivirus with genome accession number NC_005064.1 comprise or consist of: TTTGAGCAGGGCACGAAAGTGTCGGGCCTGACGCACCCGCTCCCCCGAGTCCCCTG GAAACAGGGTGGGCCTCGAAAAATCCACCGT (SEQ ID NO: 42). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 42. In some embodiments, the stabilizing sequence comprises a sequence encoded by SEQ ID NO. 42. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_027819.1. The sequence may be a DNA sequence. The sequence may be an RNA sequence. In some embodiments, the sequences from this flavivirus with genome accession number NC_027819.1 comprise or consist of: GCAGGGCAACAGGAAGAAATTCCGGGTGATTAGCCACACCCCCCGAAACGTGATTT ATATGATGACAAGAATCAGA (SEQ ID NO: 43). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 43. In some embodiments, the stabilizing sequence comprises a sequence encoded by SEQ ID NO. 43. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_034017.1. The sequence may be a DNA sequence. The sequence may be an RNA sequence. In some embodiments, the sequences from this flavivirus with genome accession number NC_034017.1 comprise or consist of: TGGGGGCAGCCGGGGGAAACCCTGGGGCTTGGCGACCTCCCCCCACAAGCCATCAT GCGAAATTAAGGCAGCCGCGAG (SEQ ID NO: 44). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 44. In some embodiments, the stabilizing sequence comprises a sequence encoded by SEQ ID NO. 44. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_034204.1. The sequence may be a DNA sequence. The sequence may be an RNA sequence. In some embodiments, the sequences from this flavivirus with genome accession number NC_034204.1 comprise or consist of: TGGGGGCAGCCGGGAGTCAAACTCCCGGGGCCTGGCGACCCCCCCCTTCCGCCTCC AAAAATTAAGGCAGCCCCGAGGGAGCTCTCCTCGGTGTGA (SEQ ID NO:45). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 45. In some embodiments, the stabilizing sequence comprises a sequence encoded by SEQ ID NO. 45. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_020902.1. The sequence may be a DNA sequence. The sequence may be an RNA sequence. In some embodiments, the sequences from this flavivirus with genome accession number NC_020902.1 comprise or consist of: CAAGGGCAGGTTGAAAGGGGCTTGGCGACCCCCCCCTAACCAGCCACGCGCACTGT GCGTGCGC (SEQ ID NO: 46). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 46. In some embodiments, the stabilizing sequence comprises a sequence encoded by SEQ ID NO. 46. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_020902.1. The sequence may be a DNA sequence. The sequence may be an RNA sequence. In some embodiments, the sequences from this flavivirus with genome accession number NC_020902.1 comprise or consist of: CCGGGGCAGGTTGAAAGGGGTTTCACGCACCCCCCCCTACCAGTGCCGTCGTTGGTA ACGCG (SEQ ID NO: 47). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 47. In some embodiments, the stabilizing sequence comprises a sequence encoded by SEQ ID NO. 47. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number MF438044.1. The sequence may be a DNA sequence. The sequence may be an RNA sequence. In some embodiments, the sequences from this flavivirus with genome accession number MF438044.1 comprise or consist of: CAAGGGCAGGTTGAAAGGGGCTTGGCGACCCCCCCCTAACTAGCCACGCGCACTGA TGTGCGCGG (SEQ ID NO: 48). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 48. In some embodiments, the stabilizing sequence comprises a sequence encoded by SEQ ID NO. 48. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number MF438044.1. The sequence may be a DNA sequence. The sequence may be an RNA sequence. In some embodiments, the sequences from this flavivirus with genome accession number MF438044.1 comprise or consist of: CCGGGGCAGGTTGAAAGGGGCATCACGCACCCCCCCCTACCAGTGCCGTCGTTGGT AACGCG (SEQ ID NO: 49). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 49. In some embodiments, the stabilizing sequence comprises a sequence encoded by SEQ ID NO. 49. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number AF070476.1. The sequence may be a DNA sequence. The sequence may be an RNA sequence. In some embodiments, the sequences from this flavivirus with genome accession number AF070476.1 comprise or consist of: ACGGGCAAGGAGCGCAAGCTGGGGCTTTCCGACCCCCCCCCCCAGGACGATTCCCC GCTTGGTAAAAAGGGCCAGGCCA (SEQ ID NO: 50). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 50. In some embodiments, the stabilizing sequence comprises a sequence encoded by SEQ ID NO. 50. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_001837.1. The sequence may be a DNA sequence. The sequence may be an RNA sequence. In some embodiments, the sequences from this flavivirus with genome accession number NC_001837.1 comprise or consist of: CTCCAGGCAGCAGCAGACGCAAGTCTGGGGGAAACGATCGCTCCTCCCTCTGCAGA TCTCTAGCTCGGATAGAGCGG (SEQ ID NO: 51). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 51. In some embodiments, the stabilizing sequence comprises a sequence encoded by SEQ ID NO. 51. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number KF234530.1. The sequence may be a DNA sequence. The sequence may be an RNA sequence. In some embodiments, the sequences from this flavivirus with genome accession number KF234530.1 comprise or consist of: ATGTGGCAAGGGGCCTGCTAACACAGGCCGGGGCTTCCTGACCCCCCCACCCCAAG GCTGTTCCCCGCTCGGTAAAAAGGGCCGGGCCA (SEQ ID NO: 52). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 52. In some embodiments, the stabilizing sequence comprises a sequence encoded by SEQ ID NO. 52. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number KT166442.1. The sequence may be a DNA sequence. The sequence may be an RNA sequence. In some embodiments, the sequences from this flavivirus with genome accession number KT166442.1 comprise or consist of: ATGAGGCAAGGAGCCTGCTAACACAGGCTGGGGCTTCCTGACCCCCCCTCCCCAAG GCGGTTCCCCGCTCGGTAAAAAGGGCCGGG (SEQ ID NO: 53). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 53. In some embodiments, the stabilizing sequence comprises a sequence encoded by SEQ ID NO. 53. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_024377.1. The sequence may be a DNA sequence. The sequence may be an RNA sequence. In some embodiments, the sequences from this flavivirus with genome accession number NC_024377.1 comprise or consist of: GTGAGGCAAGGAGACATTCCAGGAATGTCTGGGGCTTTCCGACCCCCCCTCCCCAG GACGGTTCCCCGCTGAGTAAAAAGGGCT (SEQ ID NO: 54). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 54. In some embodiments, the stabilizing sequence comprises a sequence encoded by SEQ ID NO. 54. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number KF234529.1. The sequence may be a DNA sequence. The sequence may be an RNA sequence. In some embodiments, the sequences from this flavivirus with genome accession number KF234529.1 comprise or consist of: AATGTGGCAAGGGGCCTGTCCAAGACAGGCCGGGGCTTTCCGACCCCCCACCCCCA GGACGGTTCCCCGCTCGGTAAAAAGGGCCGGGCTA (SEQ ID NO: 55). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 55. In some embodiments, the stabilizing sequence comprises a sequence encoded by SEQ ID NO. 55. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number AB008335.1. The sequence may be a DNA sequence. The sequence may be an RNA sequence. In some embodiments, the sequences from this flavivirus with genome accession number AB008335.1 comprise or consist of: TTGCGGCAAGGTCGGCCGACTGATCATCGGCTGAGGAGGTTCCCGCCCTCCCCGCCC CAGGGGTCTCCCCGCTGGGTAAAAAGGGCCCGGCCT (SEQ ID NO: 56). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 56. In some embodiments, the stabilizing sequence comprises a sequence encoded by SEQ ID NO. 56. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_001710.1. The sequence may be a DNA sequence. The sequence may be an RNA sequence. In some embodiments, the sequences from this flavivirus with genome accession number NC_001710.1 comprise or consist of: TTGCGGCAAGGTCTGGTGACTGATCATCACCGGAGGAGGTTCCCGCCCTCCCCGCCC CAGGGGTCTCCCCGCTGGGTAAAAAGGGCCCGGCCT (SEQ ID NO: 57). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 57. In some embodiments, the stabilizing sequence comprises a sequence encoded by SEQ ID NO. 57. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number AB018667.1. The sequence may be a DNA sequence. The sequence may be an RNA sequence. In some embodiments, the sequences from this flavivirus with genome accession number AB018667.1 comprise or consist of: TTGCGGCAAGGTCGGGCGACTGATCATCGCCTGAGGAGGTTCCCGCCCTCCCCGCCC CAGGGGTCTCCCCGCTGGGTAAAAAGGGCCCGGCCT (SEQ ID NO: 58). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 58. In some embodiments, the stabilizing sequence comprises a sequence encoded by SEQ ID NO. 58. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_027998.2. The sequence may be a DNA sequence. The sequence may be an RNA sequence. In some embodiments, the sequences from this flavivirus with genome accession number NC_027998.2 comprise or consist of: AGGCAGGAGGTGAAGTCAGCTGTACCCACGGCTGGCTGAAACCGGGGCTTGACGAC CCCCCCTATCCGAGTTGGGCAAGGTAACATCACGGGTGTGACGACCCC (SEQ ID NO: 59). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 59. In some embodiments, the stabilizing sequence comprises a sequence encoded by SEQ ID NO. 59. The stabilizing sequence may be transcribed into an RNA molecule. In some embodiments, the stabilizing sequence that protects the trans-splicing molecule from exonucleases that act in the 5′ to 3′ direction comprises or consists of sequences from a flavivirus with genome accession number KC796093.1. The sequence may be a DNA sequence. The sequence may be an RNA sequence. In some embodiments, the sequences from this flavivirus with genome accession number KC796093.1 comprise or consist of: TTCCGGCAAGGTGCGCCGGGGGGGCCTTCACGGGCCCTTCTAGCGCAGGGGTTTGA GACACCCCCCGCCCCACTOCTTOCCAGGGTTGGCAACCTGGGTC (SEQ ID NO: 60). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 60. In some embodiments, the stabilizing sequence comprises a sequence encoded by SEQ ID NO. 60. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number KC796084.1. The sequence may be a DNA sequence. The sequence may be an RNA sequence. In some embodiments, the sequences from this flavivirus with genome accession number KC796084.1 comprise or consist of: GTAAGGCAAGGTGTGCACTGGCAGCCTTAACGGGCTGTTGGGGCGCAGGGGCTTGA GCACCCCCCTTCCCCACTCCCAGCGGGGCTTGGCAACCCTG (SEQ ID NO: 61). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 61. In some embodiments, the stabilizing sequence comprises a sequence encoded by SEQ ID NO. 61. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number KC796084.1. The sequence may be a DNA sequence. The sequence may be an RNA sequence. In some embodiments, the sequences from this flavivirus with genome accession number KC796084.1 comprise or consist of: GTTAGCAAGGCGCTGTCATGGTGCTTCTAACGAGGCATGAGGCAGCGGGGGCGTGA GAACCCCCCTTCCCCACTCCGGCGTGTAGATTGGCAATCTTGCGCT (SEQ ID NO: 62). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 62. In some embodiments, the stabilizing sequence comprises a sequence encoded by SEQ ID NO. 62. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number KC796084.1. The sequence may be a DNA sequence. The sequence may be an RNA sequence. In some embodiments, the sequences from this flavivirus with genome accession number KC796084.1 comprise or consist of: ACTTGGCAAGGCACCCCCTGGCTAGTGGCTGCACTTAACTGAGTGGCCGGCTAGTCG GTGGGTGGGGGCACGTGACTCCCCCTTCCCCACACCGGCGGCGGCCGTAAAACGCC CC (SEQ ID NO: 63). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 63. In some embodiments, the stabilizing sequence comprises a sequence encoded by SEQ ID NO. 63. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_038435.1. The sequence may be a DNA sequence. The sequence may be an RNA sequence. In some embodiments, the sequences from this flavivirus with genome accession number NC_038435.1 comprise or consist of: TCCTGGCAAGGGCTACTCTGAAAGCTGCTAACGTGGTGATCGGCGTAGCGGGGCGT GAGGAACCCCCCACCCCACTCCTGGGTCAGCTTGGTAACTGGCCCA (SEQ ID NO: 64). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 64. In some embodiments, the stabilizing sequence comprises a sequence encoded by SEQ ID NO. 64. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_038434.1. The sequence may be a DNA sequence. The sequence may be an RNA sequence. In some embodiments, the sequences from this flavivirus with genome accession number NC_038434.1 comprise or consist of: GCGGGCAGAGACTGGGCCTCGCTGGCGCGTGAGCTCCTGTGGGATCCGGCCTGTTCC ATGCCACCTGTTCTCGATCAGGGGGAGGGGATCTTACCCCCTGAACTGT (SEQ ID NO: 65). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 65. In some embodiments, the stabilizing sequence comprises a sequence encoded by SEQ ID NO. 65. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number KC796079.1. The sequence may be a DNA sequence. The sequence may be an RNA sequence. In some embodiments, the sequences from this flavivirus with genome accession number KC796079.1 comprise or consist of: GTAAGGCAAGGTGTGCACTGGCAGCCTTAACGGGCTGTTGGGACACAGGGGCTTGA GCACCCCCCTTCCCCACTCCCAGCGGGGCTTGGCAACCCCCC (SEQ ID NO: 66). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 66. In some embodiments, the stabilizing sequence comprises a sequence encoded by SEQ ID NO. 66. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_021154.1. The sequence may be a DNA sequence. The sequence may be an RNA sequence. In some embodiments, the sequences from this flavivirus with genome accession number NC_021154.1 comprise or consist of: TTGGGGCAGGCACGCTTGCGTGGGGGAGTTGCGCCCCCCCCCAGCCAGCACTCGTC ATGATGTGTCGGATGCCAGTGATAGGCAGCCTC (SEQ ID NO: 67). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 67. In some embodiments, the stabilizing sequence comprises a sequence encoded by SEQ ID NO. 67. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_038433.1. The sequence may be a DNA sequence. The sequence may be an RNA sequence. In some embodiments, the sequences from this flavivirus with genome accession number NC_038433.1 comprise or consist of: GCCCGGCAAGGTTAACAGGGGGAGTAGTGCCCCCCCCGCCCCAACTCGGGTAGCGC GTACGCTC (SEQ ID NO: 68). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by

SEQ ID NO: 68. In some embodiments, the stabilizing sequence comprises a sequence encoded by SEQ ID NO. 68. The stabilizing sequence may be transcribed into an RNA molecule.

In some embodiments, the stabilizing sequence that protects the trans-splicing molecule from exonucleases that act in the 5° to 3′ direction comprises or consists of sequences from a flavivirus with genome accession number NC_038433.1. The sequence may be a DNA sequence. The sequence may be an RNA sequence. In some embodiments, the sequences from this flavivirus with genome accession number NC_038433.1 comprise or consist of: TCCAAGGCAACAGGCTTCGGCCGGGGGAGTAGCGCCCCCCCCTTTGTGAGCTCGTA ACCCCCTTTTGGGGCT (SEQ ID NO: 69). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 69. In some embodiments, the stabilizing sequence comprises a sequence encoded by SEQ ID NO. 69. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_030291.1. The sequence may be a DNA sequence. The sequence may be an RNA sequence. In some embodiments, the sequences from this flavivirus with genome accession number NC_030291.1 comprise or consist of; ACATGGCAAGGGTGCTTCGGCATGGGGGAGTAGCGTCCCTCCCACCCCAGCGAGGC TGCAAGCCTAT (SEQ ID NO: 70). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 70. In some embodiments, the stabilizing sequence comprises a sequence encoded by SEQ ID NO. 70. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number HM047196.1. The sequence may be a DNA sequence. The sequence may be an RNA sequence. In some embodiments, the sequences from this flavivirus with genome accession number HM047196.1 comprise or consist of: ACCTGGCAACAGCCCCGTCGGGGCGGGGGAGTAGCGCCCCCCCCAGTGTGAGOGAG GTGGGAAACCACCTA (SEQ ID NO: 71). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 71. In some embodiments, the stabilizing sequence comprises a sequence encoded by SEQ ID NO. 71. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_038437.1. The sequence may be a DNA sequence. The sequence may be an RNA sequence. In some embodiments, the sequences from this flavivirus with genome accession number NC_038437.1 comprise or consist of: CAAGGGCAAGGTGTCTTGCGAGACAGGGGCTTAACGCACCCCCCCCCCCAGTGAGG GGGGCTGATCCCCCA (SEQ ID NO: 72). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 72. In some embodiments, the stabilizing sequence comprises a sequence encoded by SEQ ID NO. 72. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_021154.1. The sequence may be a DNA sequence. The sequence may be an RNA sequence. In some embodiments, the sequences from this flavivirus with genome accession number NC_021154.1 comprise or consist of: ACCGGGCAAGGGCTCACGCGGAGTGTGACAAGCTCCCCCCCCCAGTCCATGGCCGT GGATCGGCTC (SEQ ID NO: 73). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 73. In some embodiments, the stabilizing sequence comprises a sequence encoded by SEQ ID NO. 73. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_025677.1. The sequence may be a DNA sequence. The sequence may be an RNA sequence. In some embodiments, the sequences from this flavivirus with genome accession number NC_025677.1 comprise or consist of: GAATAGGCAGGGAGGAGTCCAAGAACCGTCTCGGGGACTCTTTGGGGCTTGACGAA CCCCCCTACCCGAGTCTATATTCAGTGGCTGGAACC (SEQ ID NO: 74). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 74. In some embodiments, the stabilizing sequence comprises a sequence encoded by SEQ ID NO. 74. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number KY370101.1. The sequence may be a DNA sequence. The sequence may be an RNA sequence. In some embodiments, the sequences from this flavivirus with genome accession number KY370101.1 comprise or consist of: AAAGGCAGGGAGAGGCTTAAGAACCGTCTCGGGAGCCTCTTGGGGCTTGACGAACC CCCCAACCCGAGTCAAGTCCTTCAACAGTACCGTTTCGAG (SEQ ID NO: 75). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 75. In some embodiments, the stabilizing sequence comprises a sequence encoded by SEQ ID NO. 75. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_038964.1. The sequence may be a DNA sequence. The sequence may be an RNA sequence. In some embodiments, the sequences from this flavivirus with genome accession number NC_038964.1 comprise or consist of: TGAAAGGGGCAAGTGGCCGTATAGGCTGGGGCGATCGCCGTACCCCCCCITTACCA GGCGCCTCAACCCCATGTACCATGGGGTT (SEQ ID NO: 76). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 76. In some embodiments, the stabilizing sequence comprises a sequence encoded by SEQ ID NO. 76. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number KY370100.1. The sequence may be a DNA sequence. The sequence may be an RNA sequence. In some embodiments, the sequences from this flavivirus with genome accession number KY370100.1 comprise or consist of: ACACGGCAAGGTGCTAGAAGCTGAAACCGACTCGGAGCTCTAGCAGGGGGACTGGC GACCCTCCCGCCCCAGCTGGCCTCTGGCAGAAACGACTCGTGCCATT (SEQ ID NO: 77). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 77. In some embodiments, the stabilizing sequence comprises a sequence encoded by SEQ ID NO. 77. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number MH282908.1. The sequence may be a DNA sequence. The sequence may be an RNA sequence. In some embodiments, the sequences from this flavivirus with genome accession number MH282908.1 comprise or consist of: AAGAGGCAAGGGGAGGCAGCTAGCCTCCGGGGCCTGACGACCCCCCTTCCCCAGTC ATTGAAGGCAAGGGGCTGC (SEQ ID NO: 78). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 78. In some embodiments, the stabilizing sequence comprises a sequence encoded by SEQ ID NO. 78. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number MH282908.1. The sequence may be a DNA sequence. The sequence may be an RNA sequence. In some embodiments, the sequences from this flavivirus with genome accession number MH282908.1 comprise or consist of: AAAGGGCAAGGCACCACACGGCTAGTGTGGTGAGGGATTGACAACCCCTCCTCCCC AGTCGGCATGAACTT (SEQ ID NO: 79). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 79. In some embodiments, the stabilizing sequence comprises a sequence encoded by SEQ ID NO. 79. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number MH282908.1. The sequence may be a DNA sequence. The sequence may be an RNA sequence. In some embodiments, the sequences from this flavivirus with genome accession number MH282908.1 comprise or consist of: AAAGGGCAAGGCACCACATGGAAATGTGGTGAGGGTTTGACCTCCCCTCCCCCCCA GTCAACCATACATAAAACTTGAAAAACACATATTGGTACT (SEQ ID NO: 80). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 80. In some embodiments, the stabilizing sequence comprises a sequence encoded by SEQ ID NO. 80. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_001655.1. The sequence may be a DNA sequence. The sequence may be an RNA sequence. In some embodiments, the sequences from this flavivirus with genome accession number NC_001655.1 comprise or consist of: CAGCGGCAACAGGGGAGACCCCGGGCTTAACGACCCCGCCGATGTGAGTTTGGCGA CCATGGTGGATCAG (SEQ ID NO: 81). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 81. In some embodiments, the stabilizing sequence comprises a sequence encoded by SEQ ID NO. 81. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_031950.1. The sequence may be a DNA sequence. The sequence may be an RNA sequence. In some embodiments, the sequences from this flavivirus with genome accession number NC_031950.1 comprise or consist of: CAATGGCAACAGCACTCTCTTAGGTGCGGGGTATGGCGAACCCCCCAATGTGAGCT CCTCCCCGGATGGGGCG (SEQ ID NO: 82). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 82. In some embodiments, the stabilizing sequence comprises a sequence encoded by SEQ ID NO. 82. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_038430.1. The sequence may be a DNA sequence. The sequence may be an RNA sequence. In some embodiments, the sequences from this flavivirus with genome accession number NC_038430.1 comprise or consist of: TTCGGGCAAGGCATGCGAGATAAAAAGGGTCTCGTATGAGGGCGTGGCAACCCCTC CCCCCCAGCTGCGGCGGCACAAAAGCGTCTCGCGTGTCGTC (SEQ ID NO: 83). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 83. In some embodiments, the stabilizing sequence comprises a sequence encoded by SEQ ID NO. 83. The stabilizing sequence may be transcribed into an RNA molecule.

In some embodiments, the stabilizing sequence that protects the trans-splicing molecule from exonucleases that act in the 5′ to 3° direction comprises or consists of sequences from a flavivirus with genome accession number NC_040815.1. The sequence may be a DNA sequence. The sequence may be an RNA sequence. In some embodiments, the sequences from this flavivirus with genome accession number NC_040815.1 comprise or consist of: TAGCGGCAGGGAGCAGGGTAGACCAACCTGCAGGGGCTTGACGACCCCCCCGTCCC GAGTCAGCCAGGAGGCAGAAGCGACTCGC (SEQ ID NO: 84). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 84. In some embodiments, the stabilizing sequence comprises a sequence encoded by SEQ ID NO. 84. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number KY370094.1. The sequence may be a DNA sequence. The sequence may be an RNA sequence. In some embodiments, the sequences from this flavivirus with genome accession number KY370094.1 comprise or consist of: TGAGGGCAGGATAGGCCAAGAATCGTCTCGAGGCTGATTGGGGCTTCACGCACCCC CCCATCCGAGTGCCTCTCATCTTCCAAAACCGTCTCGGGGGAGATGAGAC (SEQ ID NO: 85). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 85. In some embodiments, the stabilizing sequence comprises a sequence encoded by SEQ ID NO. 85. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number KX905133.1. The sequence may be a DNA sequence. The sequence may be an RNA sequence. In some embodiments, the sequences from this flavivirus with genome accession number KX905133.1 comprise or consist of; AAAGGGCAGCGGTGCCGGACGGCATTGGGGCTTGGCGACCCCCCCCACGCGAGCTA CCCACCATTGGTGGGTTC (SEQ ID NO: 86). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 86. In some embodiments, the stabilizing sequence comprises a sequence encoded by SEQ ID NO. 86. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_021153.1. The sequence may be a DNA sequence. The sequence may be an RNA sequence. In some embodiments, the sequences from this flavivirus with genome accession number NC_021153.1 comprise or consist of: CAGGGGCAGAGGCCGGTTGATTATCATTGCAGCCGTAGGGGCTTGGCGACCCCCCC CCCTCGAGCCAGCCTTTCAACAAAACCGTCTCGGGTTGGAAGG (SEQ ID NO: 87), In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 87. In some embodiments, the stabilizing sequence comprises a sequence encoded by SEQ ID NO. 87. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number NC_038428.1. The sequence may be a DNA sequence. The sequence may be an RNA sequence. In some embodiments, the sequences from this flavivirus with genome accession number NC_038428.1 comprise or consist of: ACACTGCAACAGGGGAAACCCGGGGATTTCCGATCCCCCAGATGTGAGGAGGCTGG TTGCCTAACAACCTG (SEQ ID NO: 88). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 88. In some embodiments, the stabilizing sequence comprises a sequence encoded by SEQ ID NO. 88. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number KJ412989. The sequence may be a DNA sequence. The sequence may be an RNA sequence. In some embodiments, the sequences from this flavivirus with genome accession number KJ412989 comprise or consist of: CTCCCGTAAGGAAAGCGCAAGCTTTGAGCATTGACAACGCTCCGGCCCCAGTCCCCC AGGTTATGGAGGAATAACCC (SEQ ID NO: 89). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 89. In some embodiments, the stabilizing sequence comprises a sequence encoded by SEQ ID NO. 89. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number MN242370. The sequence may be a DNA sequence. The sequence may be an RNA sequence. In some embodiments, the sequences from this flavivirus with genome accession number MN242370 comprise or consist of: AAGGGGCAGGATATGGACAGAACCGTCTCGGGTCCAGAAGGGGCTTGGCGACCCCC CCCATCCGAGCCACCCCTCTAGGAAGACCGTCTCGGCCTAGAGG (SEQ ID NO: 90). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 90. In some embodiments, the stabilizing sequence comprises a sequence encoded by SEQ ID NO. 90. The stabilizing sequence may be transcribed into an RNA molecule.

In some embodiments, the stabilizing sequence that protects the trans-splicing molecule from exonucleases that act in the 5′ to 3′ direction comprises or consists of sequences from a flavivirus with genome accession number MH824541. The sequence may be a DNA sequence. The sequence may be an RNA sequence. In some embodiments, the sequences from this flavivirus with genome accession number MH824541 comprise or consist of: AAGGGGCAGCCATGCCGCAGAACCGTCTCGGGGGGCAAGGGGCTTAGCGACCCCCC CCTGGCGAGCTGTATGAGTGTGATAAGGGCGACATAGC (SEQ ID NO: 91). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 91. In some embodiments, the stabilizing sequence comprises a sequence encoded by SEQ ID NO. 91. The stabilizing sequence may be transcribed into an RNA molecule.

In some embodiments, the stabilizing sequence that protects the trans-splicing molecule from exonucleases that act in the 5′ to 3′ direction comprises or consists of sequences from Tamana Bat virus (AF346759.1). The sequence may be a DNA sequence. The sequence may be an RNA sequence. In some embodiments, the sequences from Tamana Bat virus comprise or consist of: AF346759.1, pos: 10305-10380: TTTGGGCAAGGTGCAGGTTAGCTGCAGGGGCTTGAAAAACCCCCCCCCCCATTCAA GACTTTTAGTGCATTAGTT (SEQ ID NO: 103). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 103. In some embodiments, the stabilizing sequence comprises a sequence encoded by SEQ ID NO. 103. The stabilizing sequence may be transcribed into an RNA molecule.

Stabilizing Sequences that Protect the 3′ End of Trans-Splicing RNAs

The present disclosure provides a nucleic acid molecule comprising one or more stabilizing sequences to prevent or attenuate degradation of the nucleic acid molecule. In some embodiments, a DNA or RNA molecule provided herein comprises one or more stabilizing sequences to prevent degradation of the nucleic acid molecule. The DNA molecule comprising one or more stabilizing sequences may encode an RNA molecule comprising one or more stabilizing sequences. The DNA molecule comprising one or more stabilizing sequences may be transcribed into an RNA molecule comprising one or more stabilizing sequences. The degradation may be caused by, e.g., the activity of exonucleases. In some embodiments of the compositions of the present disclosure, there may be a stabilizing sequence that prevents or attenuates the activity of exonucleases that act in the 3′ to 5′ direction on RNA. In some instances, the prevention or attenuation of the activity of exonucleases increases the effectiveness of the trans-splicing molecule,

In some embodiments of the compositions of the present disclosure, the stabilizing sequence forms a tertiary structure. In some embodiments of the compositions of the present disclosure, the tertiary structure is a triplex.

In some embodiments, the stabilizing sequence is DNA. In some embodiments, the stabilizing sequence is RNA. In some embodiments, the DNA molecule encodes a gene or portion thereof to be transcribed. In some embodiments, the gene or portion thereof corresponds to a missing sequence in a target RNA. In some embodiments, the DNA molecule is transcribed into a messenger RNA molecule, and the messenger RNA molecule then selectively binds and promotes a trans-splicing reaction with a target RNA.

In some embodiments, the Stabilizing Domain forms an RNA triplex that blocks 3′-5′ exonuclease activity and is derived or isolated from a vertebrate gene or microbial genome selected from the group consisting of: MALAT1 [ENSG00000251562], NEAT1 [ENSG00000245532], Turnip yellow mosaic virus genome, Kaposi's sarcoma-associated herpesvirus genome, TER telomerase-associated RNA [ENSG00000270141], SAM-II bacterial riboswitch.

In some embodiments, the stabilizing sequence that protects the trans-splicing molecule from exonucleases that act in the 3′ to 5′ direction comprises or consists of sequences from the MALAT1 gene. The sequence may be a DNA sequence. The sequence may be an RNA sequence. In some embodiments, the sequences from the MALAT1 gene comprise or consist of: AAGCTGATCTCCAATGCTCTTCAGTAGGGTCATGAAGGTTTTTCTTTTCCTGAGAAA ACAACACGTATTGTTTTCTCAGGTTTTGCTTTTTGGCCTTTTTCTAGCTTAAAAAAAA AAAAAGCAAAAGATGCTGGTGGTTGGCACTCCTGGTTTCCAGGACGGGGTTCAAAT (SEQ ID NO: 92). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 92. In some embodiments, the stabilizing sequence comprises a sequence encoded by SEQ ID NO. 92. The sequence may be transcribed into an RNA molecule.

In some embodiments, the stabilizing sequence that protects the trans-splicing molecule from exonucleases that act in the 3′ to 5′ direction comprises or consists of sequences from rhesus rhadinovirus. The sequence may be a DNA sequence. The sequence may be an RNA sequence. In some embodiments, the sequences from rhesus rhadinovirus comprise or consist of: CGTTTGTGTTGGTTTTTATGACCAGCTTGGTACAAAACCTGCTGGTGATTTTTTACCC AACAAATATTA (SEQ ID NO: 93). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 93. In some embodiments, the stabilizing sequence comprises a sequence encoded by SEQ ID NO. 93. The stabilizing sequence may be transcribed into an RNA molecule.

In some embodiments, the stabilizing sequence that protects the trans-splicing molecule from exonucleases that act in the 3′ to 5′ direction comprises or consists of sequences from Equine Herpesvirus 2. The sequence may be a DNA sequence. The sequence may be an RNA sequence. In some embodiments, the sequences from Equine Herpesvirus 2 comprise or consist of: AAGAATATTITTAAAGACTTTTTTCCCCAACCTCTGGGTTGGGTTTTTTCTCITTAAA ATATTCAATA (SEQ ID NO: 94). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 94. In some embodiments, the stabilizing sequence comprises a sequence encoded by SEQ ID NO. 94. The stabilizing sequence may be transcribed into an RNA molecule.

In some embodiments, the stabilizing sequence that protects the trans-splicing molecule from exonucleases that act in the 3′ to 5′ direction comprises or consists of sequences from Kaposi's sarcoma-associated herpesvirus. The sequence may be a DNA sequence. The sequence may be an RNA sequence. In some embodiments, the sequences from Kaposi's sarcoma-associated herpesvirus comprise or consist of: TGTTTTGTGTTTTGGCTGGGTTTTTCCTTOTTCGCACCGGACACCTCCAGTGACCAGA CGGCAAGGTTTTTATCCCAGTGTATATT (SEQ ID NO: 95). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 95. In some embodiments, the stabilizing sequence comprises a sequence encoded by SEQ ID NO. 95. The stabilizing sequence may be transcribed into an RNA molecule.

In some embodiments, the stabilizing sequence that protects the trans-splicing molecule from exonucleases that act in the 3′ to 5′ direction comprises or consists of sequences from Plautia stali intestine virus. The sequence may be a DNA sequence. The sequence may be an RNA sequence. In some embodiments, the sequences from Plautia stali intestine virus comprise or consist of: ATTGOCAGTAGAGTTTTTCCCCAGGGAGCTTCACTGTCTGGGTTTTCTCTACT (SEQ ID NO: 96). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 96. In some embodiments, the stabilizing sequence comprises a sequence encoded by SEQ ID NO. 96. The stabilizing sequence may be transcribed into an RNA molecule.

In some embodiments, the stabilizing sequence that protects the trans-splicing molecule from exonucleases that act in the 3′ to 5′ direction comprises or consists of sequences from Cotesia congregata bracovirus. The sequence may be a DNA sequence. The sequence may be an RNA sequence. In some embodiments, the sequences from Cotesia congregata bracoviruscomprise or consist of: TTCATCAAGGAGGTTTTTTCCCAGCCTAGCTGGGTTTTCCTCCTTTGGGGACA (SEQ ID NO: 97). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 97. In some embodiments, the stabilizing sequence comprises a sequence encoded by SEQ ID NO. 97. The stabilizing sequence may be transcribed into an RNA molecule.

In some embodiments, the stabilizing sequence that protects the trans-splicing molecule from exonucleases that act in the 3′ to 5′ direction comprises or consists of sequences from Cotesia sesamiae bracoviruses. The sequence may be a DNA sequence. The sequence may be an RNA sequence. In some embodiments, the sequences from Cotesia sesamiae bracoviruses comprise or consist of: TTTTTCGAGGAGGTTTTTTCCTAGCACCACTAGGTTTTCCTCCTCTGGGAAC (SEQ ID NO: 98). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 98. In some embodiments, the stabilizing sequence comprises a sequence encoded by SEQ ID NO. 98. The stabilizing sequence may be transcribed into an RNA molecule.

In some embodiments, the stabilizing sequence that protects the trans-splicing molecule from exonucleases that act in the 3′ to 5′ direction comprises or consists of sequences from Acanthamoeba polyphaga mimivirus. The sequence may be a DNA sequence. The sequence may be an RNA sequence. In some embodiments, the sequences from Acanthamoeba polyphaga mimivirus comprise or consist of: ATTTACTGTTGGTTTTCTTCTCTGATTTTCATAAGAACTTTTCCCAACA (SEQ ID NO: 99). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 99. In some embodiments, the stabilizing sequence comprises a sequence encoded by SEQ ID NO. 99. The stabilizing sequence may be transcribed into an RNA molecule.

Stabilizing Sequences Derived from Pseudoknots that Protect the 3′ End of Trans-Splicing RNAs

The present disclosure provides a nucleic acid molecule comprising one or more stabilizing sequences to prevent or attenuate degradation of the nucleic acid molecule. In some embodiments, a DNA or RNA molecule provided herein comprises one or more stabilizing sequences to prevent degradation of the nucleic acid molecule. The degradation may be caused by, e.g., the activity of exonucleases. The exonuclease may act in the 3′ to 5′ direction on RNA. In some embodiments, the stabilizing sequence is DNA. In some embodiments, the stabilizing sequence is RNA. In some embodiments, the DNA molecule encodes a gene or portion thereof to be transcribed. In some embodiments, the gene or portion thereof corresponds to a missing sequence in a target RNA. In some embodiments, the DNA molecule is transcribed into a messenger RNA molecule, and the messenger RNA molecule then selectively binds and promotes a trans-splicing reaction with a target RNA.

In some embodiments of the compositions of the present disclosure, the stabilizing sequence forms a tertiary structure. In some embodiments of the compositions of the present disclosure, the tertiary structure is a pseudoknot.

In some embodiments, the stabilizing sequence that protects the trans-splicing molecule from exonucleases that act in the 3′ to 5′ direction comprises or consists of sequences that form a pseudoknot derived or isolated from the list consisting of: group 1 self-splicing introns from Azoarcus or Tetrahymena or Twort, drosophila sytl pre-mRNA, human CPEB3 ribozyme, E. coli RydC gene, prokaryotic plasmids I-complex or IncL/M or ColIB/P9, Mycobacterium bovis leuA mRNA, GlmS riboswitch ribozyme, Agrobacterium tumefa-ciens metA gene, L- and c-myc genes, Human interferon gamma mRNA, Ornithine decarboxylase antizyme, Prion mRNAs (human, cattle, yeast), Human and Tetrahymena telomerase, 16S rR.NA, 16S rRNA, 18S V4 region, 23S rRNA, M1 RNA component of bacterial RNase P, Neurospora VS ribozyme, Pyrimidine nucleotide synthase ribozyme, Alcohol dehydrogenase ribozyme (1-ribox02), a ribozyme, an aptanter, foot and mouse disease virus genome, Mengovirus genome, paraechovirus 1 genome, Aichivirus genome, hepatoviridae genomes, HCV, Classical swine fever virus genome, Bovine Viral Diarrhea virus genome, Porcine teschovirus, Cricket paralysis virus-like virus genomes, Giardia lamblia virus genome, Tobacco etch virus genome, retroviridae genomes, Nidovirales genomes, Totiviridae genomes, Luteoviridae genomes, Myoviridae genomes, Listeria monocytogenes phage genome, Marine leukemia virus genome, Hepatitis C virus genome, Influenza A and B genomes, Turnip yellow mosaic virus genomes, Tobacco mosaic virus-like virus genomes, Bamboo mosaic virus genome, Strawberry chlorotic fleck-associated virus genome; potato yellow vein virus genome, Tomato bushy stunt virus genome, Turnip crinkle virus genome, Encephalomyocarditis virus genome, Enterovirus genomes, Dengue virus genome, yellow fever virus genome, Japanese encephalitis virus genomes, tick-borne encephalitis virus genome, Cauliflower mosaic virus genome, Barley yellow dwarf virus genome, Bacteriophage Qβ genome, Avian leukosis virus genome, Peach latent mosaic viroid genome, Large pospiviroidae genome, Sat C satellite RNA of Turnip crinkle virus genome, Hepatitis delta virus genome, and Marek's disease virus genome.

In some embodiments, the stabilizing sequence that protects the trans-splicing molecule from exonucleases that act in the 3′ to 5′ direction comprises or consists of sequences that a form pseudoknot from Murine leukemia virus. The sequence may be a DNA sequence. The sequence may be an RNA sequence. In some embodiments, the sequences from Marine leukemia virus comprise or consist of: GGGTCAGGAGCCCCCCCCCTGAACCCAGGATAACCCTCAAAGTCGGGGGGCAACCC (SEQ ID NO: 100). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 100. In some embodiments, the stabilizing sequence comprises a sequence encoded by SEQ ID NO. 100. The stabilizing sequence may be transcribed into an RNA molecule.

In some embodiments, the stabilizing sequence that protects the trans-splicing molecule from exonucleases that act in the 3′ to 5′ direction comprises or consists of sequences that a form pseudoknot from the evopreQ1 riboswitch aptamer. The sequence may be a DNA sequence. The sequence may be an RNA sequence. In some embodiments, the sequences from the evopreQ1 riboswitch aptamer comprise or consist of: TTGACGCGGTTCTATCTAGTTACGCGTTAAACCAACTAGAAA (SEQ ID NO: 101). In some embodiments, the stabilizing sequence comprises at least about 60%, about 65%, about 70%, about 75%, about 80%, about 85%, about 90%, about 95%, about 97.5%, about 98%, about 99%, or about 100% identity with a sequence encoded by SEQ ID NO: 101. In some embodiments, the stabilizing sequence comprises a sequence encoded by SEQ ID NO. 101. The stabilizing sequence may be transcribed into an RNA sequence.

### Intronic Domains

The present composition comprises nucleic acid comprising one or more Intronic Domains. The intronic domain may promote RNA splicing of the Replacement Domain. In some embodiments, the Intronic Domains carry binding sites that are preferentially targeted by RNA-binding proteins with disease-causing mutations. In some embodiments, the dissociation constant of these mutated RNA-binding proteins and the Intronic Domain is lower than the dissociation constant of the non-mutated RNA-binding protein and the Intronic Domain.

In some embodiments, the Intronic Domains carries binding sites that are preferentially targeted by an engineered small nuclear RNA. In some embodiments, the engineered small nuclear RNA is a modified version of U1 snRNA. In some embodiments, this modified U1 snRNA increases the trans-splicing efficiency of the trans-splicing RNA.

### Replacement Domains

The present disclosure provides compositions comprising one or more Replacement Domains. The Replacement Domain may comprise DNA or RNA. The Replacement Domain may correspond to an exonic sequence of a target RNA. The exonic sequence of the target RNA may comprise a sequence that is missing or mutated. The Replacement Domain may comprise RNA. The Replacement Domain may comprise DNA. The DNA Replacement Domain may encode an RNA Replacement Domain comprising an exonic sequence or portion thereof. Th DNA Replacement Domain may be transcribed into an RNA Replacement Domain comprising an exonic sequence or portion thereof. The exonic sequence or portion thereof may be targeted to a target RNA to treat a mutation, e.g., a miscoded or missing sequence. The Replacement Domain may comprise RNA. The RNA molecule comprising an exonic sequence or portion thereof. The exonic sequence or portion thereof may be targeted to a target RNA to treat a mutation, e.g., a miscoded or missing sequence. Compositions comprising Replacement Domains disclosed herein include any strategies where replacement or insertion of RNA sequences can be an effective therapy. Replacement Domains include, without limitation, sequences derived or isolated from the following genes (with gene accession IDs in brackets and associated diseases in parentheses) such as TNFRSF13B [ENSG00000240505] (common variable immune deficiency); ADA, CECR1 [ENSG00000196839, ENSG00000093072] (Adenosine deaminase deficiency); IL2RG [ENSG00000147168] (X-linked severe combined immunodeficiency); HBB [ENSG00000244734] (Beta-thassalemia); HBA1, HBA2 [ENSG00000206172, ENSG00000188536] (alpha-thassalemia); U2AF1 [ENSG00000160201] (myelodysplastic syndrome); SOD1, TARDBP, FUS, MATR3, SOD1, C9ORF72 [ENSG00000142168, ENSG00000120948, ENSG00000089280, ENSG00000015479, ENSG00000142168, ENSG00000147894] (Amyotrophic lateral sclerosis); MAPT, PGRN [ENSG00000186868, ENSG00000030582] (Frontotemporal dementia with parkinsonism); CDH23, MYO7A, USH2A [ENSG00000107736, ENSG00000137474, ENSG00000042781] (Usher's syndrome); GALC [ENSG00000054983] (Krabbe disease); SMPD1, NPC1, NPC2 [ENSG00000166311, ENSG00000141458, ENSG00000119655] (Niemann Pick disease); PRNP [ENSG00000171867] (prion disease); SCN1A [ENSG00000144285] (Dravet syndrome); PINK1, ATPGAP2 [ENSG00000158828] (early-onset Parkinson's disease); ATXN1, ATXN2, ATXN3, PLEKHG4, SPTBN2, CACNA1A, ATXN7, TTBK2, PPP2R2B, KCNC3, PRKCG, ITPR1, TBP, KCND1, FGF14 [ENSG00000124788, ENSG00000204842, ENSG00000066427, ENSG00000196155, ENSG00000173898, ENSG00000141837, ENSG00000163635, ENSG00000128881, ENSG00000156475, ENSG00000131398, ENSG00000126583, ENSG00000150995, ENSG00000112592, ENSG00000102057, ENSG00000102466] (spinocerebellar ataxias); SCN1A, SCN2A, CACNA1A, GRIN2B, GRIN2A, MECP2, FOXG1, SLC6A1, PRRT2, PTEN, KCNQ2, KCNQ3, STARD7, CLRN1 [ENSG00000144285, ENSG00000136531, ENSG00000141837, ENSG00000273079, ENSG00000183454, ENSG00000169057, ENSG00000176165, ENSG00000157103, ENSG00000167371, ENSG00000171862, ENSG00000075043, ENSG00000184156, ENSG00000084090, ENSG00000163646] (genetic epilepsy disorders); ATM [ENSG00000149311] (Ataxia-telangiectasia); GLB1 [ENSG00000170266] (GM1 gangliosidosis); GBA [ENSG00000177628] (Gaucher disease); GM2A [ENSG00000196743] (GM2 gangliosidosis); UBE3A [ENSG00000114062] (Angelman syndrome); SLC2A1 [ENSG00000117394] (glucose transporter deficiency type 1); LAMP2 [ENSG00000005893] (Danon disease); GLA [ENSG00000102393] (Fabry disease); PKD1, PKD2 [ENSG00000008710, ENSG00000118762] (Autosomal dominant polycystic kidney disease); GAA [ENSG00000171298] (Pompe disease); PCSK9, LDLR, APOB, APOE [ENSG00000169174, ENSG00000130164, ENSG00000084674, ENSG00000130203] (Familial hypercholesterolemia); MYOC, OPTN, TBK1, WDR36, CYPIB1 [ENSG00000034971, ENSG00000123240, ENSG00000183735, ENSG00000134987, ENSG00000138061] (Open Angle Glaucoma); IDUA [ENSG00000127415] (Hurler syndrome or Mucopolysaccharidosis 1); IDS [ENSG00000010404] (Hunter syndrome or Mucopolysaccharidosis 2); CLN3 [ENSG00000188603] (Batten disease); DMD [ENS000000198947] (Duchenne muscular dystrophy); LMNA [ENSG00000160789] (Limb-girdle muscular dystrophy type 1B); DYSF [ENSG00000135636] (Limb-girdle muscular dystrophy type 2B); SGCA [ENSG00000108823] (Limb-girdle muscular dystrophy type 2D); SGCB [ENSG00000163069] (Limb-girdle muscular dystrophy type 2E); SGCG [ENSG00000102683] (Limb-girdle muscular dystrophy type 2C); SGCD [ENSG00000170624] (Limb-girdle muscular dystrophy type 2F); DUX4 [ENSG00000260596] (Facioscapulohumeral muscular dystrophy); F9 [ENSG00000101981] (Hemophilia B); F8 [ENSG00000185010] (Hemophilia A); USHA2A, RPGR, RP2, RHO, PRPF31, USH1F, PRPF3, PRPF6 [ENSG00000156313, ENSG00000102218, ENSG00000163914, ENSG00000105618, ENSG00000150275, ENSG00000117360, ENSG00000101161] (Retinitis pigmentosa); CFTR [ENSG00000001626] (cystic fibrosis); GJB2, GJB6, STRC, DENA1, WFS1 [ENSG00000165474, ENSG00000121742, ENSG00000242866, ENSG00000131504, ENSG00000109501] (autosomal dominant hearing impairment); POU3F3 [ENSG00000198914] (nonsyndromic hearing loss).

In some embodiments, the Replacement Domain is codon optimized.

In addition to sequences derived from human genes, Replacement Domains can comprise sequences derived from other organisms in order to alter the stability, translation, processing, or localization of a target RNA. Replacement Domains derived from non-human sources include, without limitation, sequences that increase protein production such as those derived or isolated from Woodchuck Hepatitis Virus (WHV) Post-transcriptional Regulatory Element (WPRE), triplex from MALAT1, the PRE of Hepatitis B virus (HPRE), and an iron response element of the form CAGYCX (Y=U or A; X=U, C, or A). In some embodiments, the Replacement Domain is derived or isolated from the Target RNA.

In some embodiments, the Replacement Domain is comprised of sequence derived or isolated from a human gene. In some embodiments of the compositions of the present disclosure, the sequence comprising the Replacement Domain has at least 50%, 55%, 60%, 65%, 70%, 75%, 80%, 87%, 90%, 95%, 97%, 99% or any percentage in between of identity with a human gene. In some embodiments, the Replacement Domain has 100% identity with a sequence derived or isolated from a human gene. In some embodiments, the Replacement Domain comprises or consists of 2 nucleotides, 5 nucleotides, 10 nucleotides, 20 nucleotides, 30 nucleotides, 40 nucleotides, 50 nucleotides, 60 nucleotides, 70 nucleotides, 80 nucleotides, 90 nucleotides, 100 nucleotides, 110 nucleotides, 120 nucleotides, 130 nucleotides, 140 nucleotides, 150 nucleotides, 160 nucleotides, 170 nucleotides, 180 nucleotides, 190 nucleotides, 200 nucleotides, 210 nucleotides, 220 nucleotides, 230 nucleotides, 240 nucleotides, 250 nucleotides, 260 nucleotides, 270 nucleotides, more than 270 nucleotides, or any number of nucleotides in between.

### Antisense Domains

The present disclosure provides nucleic acid molecules comprising one or more Antisense Domains. The Antisense Domain may comprise DNA. In some embodiments, the Antisense Domain is complementary to the target RNA. In some embodiments, the Antisense Domain binds to the target RNA. The Antisense Domain may comprise DNA. The DNA comprising an Antisense Domain may encode or be transcribed into an RNA molecule comprising an Antisense Domain. In some embodiments, the RNA molecule comprising an Antisense Domain is complementary to the target RNA. In some embodiments, the Antisense Domain binds to the target RNA. In some embodiments of the compositions of the present disclosure, a pathogenic RNA molecule is a target RNA. In some embodiments, the target RNA comprises a target sequence that is complementary to an Antisense Domain of the trans-splicing RNA of the present disclosure.

In some embodiments of the compositions and methods of the present disclosure, the target sequence comprises or consists of between 5 and 500 nucleotides. In some embodiments, the target sequence comprises or consists of between 50 and 250 nucleotides. In some embodiments, the target sequence comprises or consists of between 5 and 50 nucleotides.

In some embodiments of the compositions and methods of the present disclosure, a target sequence is contained within a single contiguous stretch of the target RNA. In some embodiments, the target sequence may consist of comprise of one or more nucleotides that are not spread among a single contiguous stretch of the target RNA.

In some embodiments of the present disclosure, an Antisense Domain of the present disclosure binds to a target sequence. In some embodiments of the present disclosure, an Antisense Domain of the present disclosure binds to a target RNA.

In some embodiments of the present disclosure, the Antisense Domain is chosen so that successful trans-splicing causes removal of micro open reading frames in the Target RNA. In this manner, the trans-splicing system removes micro open reading frames and increases the production of protein from the target RNA.

In some embodiments, the Antisense Domain is complementary to a gene (corresponding accession numbers in brackets, associated illness in parentheses) and is selected from the group consisting of: TNFRSF13B [ENSG00000240505] (common variable immune deficiency); ADA, CECR1 [ENSG00000196839, ENSG00000093072] (Adenosine deaminase deficiency); IL2RG [ENSG00000147168] (X-linked severe combined immunodeficiency); HBB [ENSG00000244734] (Beta-thassalemia); HBA1, HBA2 [ENSG00000206172, ENSG00000188536] (alpha-thassalemia); U2AF1 [ENSG00000160201] (myelodysplastic syndrome); SOD1, TARDBP, FUS, MATR3, SOD1, C9ORF72 [ENSG00000142168, ENSG00000120948, ENSG00000089280, ENSG00000015479, ENSG00000142168, ENSG00000147894] (Amyotrophic lateral sclerosis); MAPT, PGRN [ENSG00000186868, ENSG00000030582] (Frontotemporal dementia with parkinsonism); CDH23, MYO7A, USH2A [ENSG00000107736, ENSG00000137474, ENSG00000042781] (Usher's syndrome); GALC [ENSG00000054983] (Krabbe disease); SMPD1, NPC1, NPC2 [ENSG00000166311, ENSG00000141458, ENSG00000119655] (Niemann Pick disease); PRNP [ENSG00000171867] (prion disease); SCN1A [ENSG00000144285] (Dravet syndrome); PINK1, ATPGAP2 [ENSG00000158828] (early-onset Parkinson's disease); ATXN1, ATXN2, ATXN3, PLEKHG4, SPTBN2, CACNA1A, ATXN7, TTBK2, PPP2R2B, KCNC3, PRKCG, ITPR1, TBP, KCND1, FGF14 [ENSG00000124788, ENSG00000204842, ENSG00000066427, ENSG00000196155, ENSG00000173898, ENSG00000141837, ENSG00000163635, ENSG00000128881, ENSG00000156475, ENSG00000131398, ENSG00000126583, ENSG00000150995, ENSG00000112592, ENSG00000102057, ENSG00000102466] (spinocerebellar ataxias); SCN1A, SCN2A, CACNA1A, GRIN2B, GRIN2A, MECP2, FOXG1, SLC6A1, PRRT2, PTEN, KCNQ2, KCNQ3, STARD7, CLRN1 [ENSG00000144285, ENSG00000136531, ENSG00000141837, ENSG00000273079, ENSG00000183454, ENSG00000169057, ENSG00000176165, ENSG00000157103, ENSG00000167371, ENSG00000171862, ENSG00000075043, ENSG00000184156, ENSG00000084090, ENSG00000163646] (genetic epilepsy disorders); ATM [ENSG00000149311] (Ataxia-telangiectasia); GLB1 [ENSG00000170266] (GM) gangliosidosis); GBA [ENSG00000177628] (Gaucher disease); GM2A [ENSG00000196743] (GM2 gangliosidosis); UBE3A [ENSG00000114062] (Angelman syndrome); SLC2A1 [ENSG00000117394] (glucose transporter deficiency type 1); LAMP2 [ENSG00000005893] (Danon disease); GLA [ENSG00000102393] (Fabry disease); PKD1, PKD2 [ENSG00000008710, ENSG00000118762] (Autosomal dominant polycystic kidney disease); GAA [ENSG00000171298] (Pompe disease); PCSK9, LDLR, APOB, APOE [ENSG00000169174, ENSG00000130164, ENSG00000084674, ENSG00000130203] (Familial hypercholesterolemia); MYOC, OPTN, TBK1, WDR36, CYPIB1 [ENSG00000034971, ENSG00000123240, ENSG00000183735, ENSG00000134987, ENSG00000138061] (Open Angle Glaucoma); IDUA [ENSG00000127415] (Hurler syndrome or Mucopolysaccharidosis 1); IDS [ENSG00000010404] (Hunter syndrome or Mucopolysaccharidosis 2); CLN3 [ENSG00000188603] (Batten disease); DMD [ENSG00000198947] (Duchenne muscular dystrophy); LMNA [ENSG00000160789] (Limb-girdle muscular dystrophy type 1B); DYSF [ENSG00000135636] (Limb-girdle muscular dystrophy type 2B); SGCA [ENSG00000108823] (Limb-girdle muscular dystrophy type 2D); SGCB [ENSG00000163069] (Limb-girdle muscular dystrophy type 2E); SGCG [ENSG00000102683] (Limb-girdle muscular dystrophy type 2C); SGCD [ENSG00000170624] (Limb-girdle muscular dystrophy type 2F); DUX4 [ENSG00000260596] (Facioscapulohumeral muscular dystrophy); F9 [ENSG00000101981] (Hemophilia B); F8 [ENSG00000185010] (Hemophilia A); USHA2A, RPGR, RP2, RHO, PRPF31, USH1F, PRPF3, PRPF6 [ENSG00000156313, ENSG00000102218, ENSG00000163914, ENSG00000105618, ENSG00000150275, ENSG00000117360, ENSG00000101161] (Retinitis pigmentosa); CFTR [ENSG00000001626] (cystic fibrosis); GJB2, GJB6, STRC, DFNA1, WFS1 [ENSG00000165474, ENSG00000121742, ENSG00000242866, ENSG00000131504, ENSG00000109501] (autosomal dominant hearing impairment); POU3F3 [ENSG00000198914] (nonsyndromic hearing loss).

In some embodiments of the compositions of the present disclosure, the sequence comprising the Antisense Domain has at least 50%, 55%, 60%, 65%, 70%, 75%, 80%, 87%, 90%, 91%, 92%, 93%, 94%, 95%, 96%, 97%, 98%, 99% or any percentage in between of complementarity to the Target RNA sequence. In some embodiments, the Antisense Domain has 100% complementarity to the Target RNA sequence. In some embodiments, the Antisense Domain comprises or consists of 20 nucleotides, 30 nucleotides, 40 nucleotides, 50 nucleotides, 60 nucleotides, 70 nucleotides, 80 nucleotides, 90 nucleotides, 100 nucleotides, 110 nucleotides, 120 nucleotides, 130 nucleotides, 140 nucleotides, 150 nucleotides, 160 nucleotides, 170 nucleotides, 180 nucleotides, 190 nucleotides, 200 nucleotides, 210 nucleotides, 220 nucleotides, 230 nucleotides, 240 nucleotides, 250 nucleotides, 260 nucleotides, 270 nucleotides, more than 270 nucleotides, or any number of nucleotides in between the complementary to the Target RNA sequence.

### Methods of Use

The present disclosure provides a method of modifying the sequence of an RNA molecule or a protein encoded by the RNA molecule comprising contacting the composition and the RNA molecule under conditions suitable for binding and trans-splicing of one or more of the trans-splicing RNAs (or a portion thereof) to the RNA molecule. As described elsewhere herein, the efficiency of RNA trans-splicing may be defined as the fraction of a target RNA molecule that experiences a specific change in sequence composition that is mediated by trans-splicing. This efficiency measurement is a significant metric of therapeutic efficacy. In some embodiments, the efficiency of trans-splicing of the nucleic acid is increased relative to the efficiency of trans-splicing of a nucleic acid that does not comprise a stabilization domain. In some embodiments, the trans-splicing efficiency of the exonic sequence or portion thereof is increased relative to said exonic sequence of a target RNA that is not administered a stabilization domain.

The present disclosure provides a method of modifying an activity of a protein encoded by an RNA molecule comprising contacting the composition and the RNA molecule under conditions suitable for binding and trans-splicing of one or more of the trans-splicing RNAs (or a portion thereof) to the RNA molecule.

The present disclosure provides a method of modifying the sequence of an RNA molecule or a protein encoded by the RNA molecule with 15% or more efficiency, wherein the method comprises contacting the composition and the RNA molecule under conditions suitable for binding and trans-splicing of one or more of the trans-splicing RNAs (or a portion thereof) to the RNA molecule.

The present disclosure provides a method of modifying the sequence of an RNA molecule or a protein encoded by the RNA molecule with 20% or more efficiency, wherein the method comprises contacting the composition and the RNA molecule under conditions suitable for binding and trans-splicing of one or more of the trans-splicing RNAs (or a portion thereof) to the RNA molecule.

The present disclosure provides a method of modifying the sequence of an RNA molecule or a protein encoded by the RNA molecule with 30% or more efficiency, wherein the method comprises contacting the composition and the RNA molecule under conditions suitable for binding and trans-splicing of one or more of the trans-splicing RNAs (or a portion thereof) to the RNA molecule.

The present disclosure provides a method of modifying the sequence of an RNA molecule or a protein encoded by the RNA molecule with 40% or more efficiency, wherein the method comprises contacting the composition and the RNA molecule under conditions suitable for binding and trans-splicing of one or more of the trans-splicing RNAs (or a portion thereof) to the RNA molecule.

The present disclosure provides a method of modifying the sequence of an RNA molecule or a protein encoded by the RNA molecule with 50% or more efficiency, wherein the method comprises contacting the composition and the RNA molecule under conditions suitable for binding and trans-splicing of one or more of the trans-splicing RNAs (or a portion thereof) to the RNA molecule.

The present disclosure provides a method of modifying the sequence of an RNA molecule or a protein encoded by the RNA molecule with 60% or more efficiency, wherein the method comprises contacting the composition and the RNA molecule under conditions suitable for binding and trans-splicing of one or more of the trans-splicing RNAs (or a portion thereof) to the RNA molecule.

The present disclosure provides a method of modifying the sequence of an RNA molecule or a protein encoded by the RNA molecule with 70% or more efficiency, wherein the method comprises contacting the composition and the RNA molecule under conditions suitable for binding and trans-splicing of one or more of the trans-splicing RNAs (or a portion thereof) to the RNA molecule.

The present disclosure provides a method of modifying the sequence of an RNA molecule or a protein encoded by the RNA molecule with 80% or more efficiency, wherein the method comprises contacting the composition and the RNA molecule under conditions suitable for binding and trans-splicing of one or more of the trans-splicing RNAs (or a portion thereof) to the RNA molecule.

The present disclosure provides a method of modifying the sequence of an RNA molecule or a protein encoded by the RNA molecule with 90% or more efficiency, wherein the method comprises contacting the composition and the RNA molecule under conditions suitable for binding and trans-splicing of one or more of the trans-splicing RNAs (or a portion thereof) to the RNA molecule.

The present disclosure provides a method of modifying the sequence of an untranslated region of an RNA molecule, wherein the method comprises contacting the composition and the RNA molecule under conditions suitable for binding and trans-splicing of one or more of the trans-splicing RNAs (or a portion thereof) to the RNA molecule.

The present disclosure provides a method of increasing the expression of an RNA by insertion of WPRE or sequences with similar activity, wherein the method comprises contacting the composition and the RNA molecule under conditions suitable for binding and trans-splicing of one or more of the trans-splicing RNAs (or a portion thereof) to the RNA molecule.

The present disclosure provides a method of modifying the composition of a protein encoded by a target RNA, wherein the method comprises contacting the composition and a cell comprising the target RNA under conditions suitable for trans-splicing among the composition and the target RNA.

The present disclosure provides a method of modifying the composition of a target RNA with efficiency exceeding 20%, where 100% constitutes complete replacement of a chosen sequence within the target RNA, wherein the method comprises contacting the composition and a cell comprising the target RNA under conditions suitable for trans-splicing among the composition and the target RNA.

The present disclosure provides a method of modifying the composition of a protein encoded by a target RNA with efficiency at or about 20%, where 100% constitutes complete replacement of a chosen sequence within the Target RNA, wherein the method comprises contacting the composition and a cell comprising the target RNA under conditions suitable for trans-splicing among the composition and the target RNA.

The present disclosure provides a method of modifying the composition of a target RNA with efficiency at or about 60%, where 100% constitutes complete replacement of a chosen sequence within the Target RNA, wherein the method comprises contacting the composition and a cell comprising the target RNA under conditions suitable for trans-splicing among the composition and the target RNA.

The present disclosure provides a method of modifying the composition of a protein encoded by a target RNA with efficiency at or about 60% where 100% constitutes complete replacement of a chosen sequence within the Target RNA, wherein the method comprises contacting the composition and a cell comprising the target RNA under conditions suitable for trans-splicing among the composition and the target RNA.

The present disclosure provides a method of modifying the composition of a target RNA with efficiency at or about 70% where 100% constitutes complete replacement of a chosen sequence within the Target RNA, wherein the method comprises contacting the composition and a cell comprising the target RNA under conditions suitable for trans-splicing among the composition and the target RNA.

The present disclosure provides a method of modifying the composition of a protein encoded by a target RNA with efficiency at or about 70% where 100% constitutes complete replacement of a chosen sequence within the Target RNA, wherein the method comprises contacting the composition and a cell comprising the target RNA under conditions suitable for trans-splicing among the composition and the target RNA.

The present disclosure provides a method of modifying the composition of a target RNA with efficiency at or about 80% where 100% constitutes complete replacement of a chosen sequence within the Target RNA, wherein the method comprises contacting the composition and a cell comprising the target RNA under conditions suitable for trans-splicing among the composition and the target RNA.

The present disclosure provides a method of modifying the composition of a protein encoded by a target RNA with efficiency at or about 80% where 100% constitutes complete replacement of a chosen sequence within the Target RNA, wherein the method comprises contacting the composition and a cell comprising the target RNA under conditions suitable for trans-splicing among the composition and the target RNA.

The present disclosure provides a method of modifying the composition of a target RNA with efficiency at or about 90% where 100% constitutes complete replacement of a chosen sequence within the Target RNA, wherein the method comprises contacting the composition and a cell comprising the target RNA under conditions suitable for trans-splicing among the composition and the target RNA.

The present disclosure provides a method of modifying the composition of a protein encoded by a target RNA with efficiency at or about 90% where 100% constitutes complete replacement of a chosen sequence within the Target RNA, wherein the method comprises contacting the composition and a cell comprising the target RNA under conditions suitable for trans-splicing among the composition and the target RNA.

The present disclosure provides a method of modifying the composition of a target RNA with high efficiency, wherein the method comprises contacting the composition and a cell comprising the target RNA under conditions suitable for trans-splicing among the composition and the target RNA. In some embodiments, the cell is in vivo, in vitro, ex vivo or in situ. In some embodiments, the composition comprises a vector comprising or encoding a trans-splicing RNA molecule of the present disclosure. In some embodiments, the vector is an AAV.

The present disclosure provides a method of modifying the composition of a protein encoded by a target RNA with high efficiency, wherein the method comprises contacting the composition and a cell comprising the target RNA under conditions suitable for trans-splicing among the composition and the target RNA. In some embodiments, the cell is in vivo, in vitro, ex vivo or in situ. In some embodiments, the composition comprises a vector comprising or encoding a trans-splicing RNA molecule of the present disclosure. In some embodiments, the vector is an AAV.

The present disclosure provides a method of treating a disease or disorder, wherein the method comprises administering to a subject a therapeutically effective amount of a composition of the present disclosure.

The present disclosure provides a method of treating a disease or disorder, wherein the method comprises administering to a subject a therapeutically effective amount of a composition of the present disclosure, wherein the composition comprises a vector comprising or encoding a trans-splicing RNA molecule of the present disclosure, and wherein the composition modifies a level of expression of an RNA molecule of the present disclosure or a protein encoded by the RNA molecule.

The present disclosure provides a method of treating a disease or disorder, wherein the method comprises administering to a subject a therapeutically effective amount of a composition of the present disclosure, wherein the composition comprises a vector comprising or encoding a trans-splicing RNA molecule of the present disclosure and wherein the composition modifies an activity of a protein encoded by an RNA molecule.

In some embodiments of the compositions and methods of the present disclosure, a disease or disorder of the present disclosure includes, but is not limited to, a genetic disease or disorder. In some embodiments, the genetic disease or disorder is a single-gene disease or disorder. In some embodiments, the single-gene disease or disorder is an autosomal dominant disease or disorder, an autosomal recessive disease or disorder, an X-chromosome linked (X-linked) disease or disorder, an X-linked dominant disease or disorder, an X-linked recessive disease or disorder, a Y-linked disease or disorder or a mitochondrial disease or disorder. In some embodiments, the single-gene disease or disorder is, but not limited to, common variable immune deficiency, Adenosine deaminase deficiency, X-linked severe combined immunodeficiency, Beta-thassalemia, alpha-thassalemia, myelodysplastic syndrome, Amyotrophic lateral sclerosis, Frontotemporal dementia with parkinsonism, Usher's syndrome, Krabbe disease, Niemann Pick disease, prion disease, Dravet syndrome, early-onset Parkinson's disease, spinocerebellar ataxias, genetic epilepsy disorders, Ataxia-telangiectasia, GM1 gangliosidosis, Gaucher disease, GM2 gangliosidosis, Angelman syndrome, glucose transporter deficiency type 1, Danon disease, Fabry disease, Autosomal dominant polycystic kidney disease, Pompe disease, Familial hypercholesterolemia, Open Angle Glaucoma, Hurler syndrome or Mucopolysaccharidosis 1, Hunter syndrome or Mucopolysaccharidosis 2, Batten disease, Duchenne muscular dystrophy, Limb-girdle muscular dystrophy type 1B, Limb-girdle muscular dystrophy type 2B, Limb-girdle muscular dystrophy type 2D, Limb-girdle muscular dystrophy type 2E, Limb-girdle muscular dystrophy type 2C, Limb-girdle muscular dystrophy type 2F, Facioscapulohumeral muscular dystrophy, Hemophilia B, Hemophilia A, Retinitis pigmentosa, cystic fibrosis, autosomal dominant hearing impairment, and non-syndromic hearing loss. In some embodiments, the genetic disease or disorder is a multiple-gene disease or disorder. In some embodiments, the genetic disease or disorder is a multiple-gene disease or disorder. In some embodiments, the single-gene disease or disorder is an autosomal dominant disease or disorder including, but not limited to, Huntington's disease, neurofibromatosis type 1, neurofibromatosis type 2, Marfan syndrome, hereditary nonpolyposis colorectal cancer, hereditary multiple exostoses, Von Willebrand disease, and acute intermittent porphyria. In some embodiments, the single-gene disease or disorder is an autosomal recessive disease or disorder including, but not limited to, Albinism, Medium-chain acyl-CoA dehydrogenase deficiency, cystic fibrosis, sickle-cell disease, Tay-Sachs disease, Niemann-Pick disease, spinal muscular atrophy, and Roberts syndrome. In some embodiments, the single-gene disease or disorder is X-linked disease or disorder including, but not limited to, muscular dystrophy, Duchenne muscular dystrophy, Hemophilia, Adrenoleukodystrophy (ALD), Rett syndrome, and Hemophilia A. In some embodiments, the single-gene disease or disorder is a mitochondrial disorder including, but not limited to, Leber's hereditary optic neuropathy.

In some embodiments of the compositions and methods of the present disclosure, a disease or disorder of the present disclosure includes, but is not limited to, an immune disease or disorder. In some embodiments, the immune disease or disorder is an immunodeficiency disease or disorder including, but not limited to, B-cell deficiency, T-cell deficiency, neutropenia, asplenia, complement deficiency, acquired immunodeficiency syndrome (AIDS) and immunodeficiency due to medical intervention (immunosuppression as an intended or adverse effect of a medical therapy). In some embodiments, the immune disease or disorder is an autoimmune disease or disorder including, but not limited to, Achalasia, Addison's disease, Adult Still's disease, Agammaglobulinemia, Alopecia areata, Amyloidosis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenia purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenia purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, or Wegener's granulomatosis.

In some embodiments of the compositions and methods of the present disclosure, a disease or disorder of the present disclosure includes, but is not limited to, an inflammatory disease or disorder.

In some embodiments of the compositions and methods of the present disclosure, a disease or disorder of the present disclosure includes, but is not limited to, a metabolic disease or disorder.

In some embodiments of the compositions and methods of the present disclosure, a disease or disorder of the present disclosure includes, but is not limited to, a degenerative or a progressive disease or disorder. In some embodiments, the degenerative or a progressive disease or disorder includes, but is not limited to, amyotrophic lateral sclerosis (ALS), Huntington's disease, Alzheimer's disease, and aging.

In some embodiments of the compositions and methods of the present disclosure, a disease or disorder of the present disclosure includes, but is not limited to, an infectious disease or disorder.

In some embodiments of the compositions and methods of the present disclosure, a disease or disorder of the present disclosure includes, but is not limited to, a pediatric or a developmental disease or disorder.

In some embodiments of the compositions and methods of the present disclosure, a disease or disorder of the present disclosure includes, but is not limited to, a cardiovascular disease or disorder.

In some embodiments of the compositions and methods of the present disclosure, a disease or disorder of the present disclosure includes, but is not limited to, a proliferative disease or disorder. In some embodiments, the proliferative disease or disorder is a cancer. In some embodiments, the cancer includes, but is not limited to, Acute Lymphoblastic Leukemia (ALL), Acute Myeloid Leukemia (AML), Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Appendix Cancer, Gastrointestinal Carcinoid Tumors, Astrocytomas, Atypical Teratoid/Rhabdoid Tumor, Central Nervous System (Brain Cancer), Basal Cell Carcinoma, Bile Duct Cancer, Bladder Cancer, Bone Cancer, Ewing Sarcoma, Osteosarcoma, Malignant Fibrous Histiocytoma, Brain Tumors, Breast Cancer, Burkitt Lymphoma, Carcinoid Tumor, Carcinoma, Cardiac (Heart) Tumors, Embryonal Tumors, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Ductal Carcinoma In Situ, Embryonal Tumors, Endometrial Cancer (Uterine Cancer), Ependymoma, Esophageal Cancer, Esthesioneuroblastoma (Head and Neck Cancer), Ewing Sarcoma (Bone Cancer), Extracranial Germ Cell Tumor, Extragonadal Germ Cell Tumor, Eye Cancer, Childhood Intraocular Melanoma, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST) (Soft Tissue Sarcoma), Childhood Gastrointestinal Stromal Tumors, Germ Cell Tumors, Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Heart Tumors, Hepatocellular (Liver) Cancer, Histiocytosis, Hodgkin Lymphoma, Hypopharyngeal Cancer (Head and Neck Cancer), Intraocular Melanoma, Islet Cell Tumors, Pancreatic Neuroendocrine Tumors, Kaposi Sarcoma (Soft Tissue Sarcoma), Kidney (Renal Cell) Cancer, Langerhans Cell Histiocytosis, Laryngeal Cancer (Head and Neck Cancer), Leukemia, Lip and Oral Cavity Cancer (Head and Neck Cancer), Liver Cancer, Lung Cancer (Non-Small Cell and Small Cell), Childhood Lung Cancer, Lymphoma, Male Breast Cancer, Malignant Fibrous Histiocytoma of Bone and Osteosarcoma, Melanoma, Merkel Cell Carcinoma (Skin Cancer), Mesothelioma, Metastatic Squamous Neck Cancer with Occult Primary (Head and Neck Cancer), Midline Tract Carcinoma With NUT Gene Changes, Mouth Cancer (Head and Neck Cancer), Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Nasal Cavity and Paranasal Sinus Cancer (Head and Neck Cancer), Nasopharyngeal Cancer (Head and Neck Cancer), Neuroblastoma, Non-Hodgkin Lymphoma, Non-Small Cell Lung Cancer, Oral Cancer, Lip and Oral Cavity Cancer and Oropharyngeal Cancer, Osteosarcoma and Malignant Fibrous Histiocytoma of Bone, Ovarian Cancer, Pancreatic Cancer, Pancreatic Neuroendocrine Tumors (Islet Cell Tumors), Papillomatosis, Paraganglioma, Parathyroid Cancer, Penile Cancer, Pharyngeal Cancer (Head and Neck Cancer), Pheochromocytoma, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Rhabdomyosarcoma, Childhood (Soft Tissue Sarcoma), Salivary Gland Cancer (Head and Neck Cancer), Sarcoma, Childhood Rhabdomyosarcoma (Soft Tissue Sarcoma), Childhood Vascular Tumors (Soft Tissue Sarcoma), Ewing Sarcoma (Bone Cancer), Kaposi Sarcoma (Soft Tissue Sarcoma), Osteosarcoma (Bone Cancer), Uterine Sarcoma, Sezary Syndrome, Lymphoma, Skin Cancer, Small Cell Lung Cancer, Small Intestine Cancer, Soft Tissue Sarcoma, Squamous Cell Carcinoma of the Skin, Squamous Neck Cancer, Stomach (Gastric) Cancer, T-Cell Lymphoma, Testicular Cancer, Throat Cancer (Head and Neck Cancer), Nasopharyngeal Cancer, Oropharyngeal Cancer, Hypopharyngeal Cancer, Thymoma and Thymic Carcinoma, Thyroid Cancer, Transitional Cell Cancer of the Renal Pelvis and Ureter, Renal Cell Cancer, Urethral Cancer, Uterine Sarcoma, Vaginal Cancer, Vascular Tumors (Soft Tissue Sarcoma), Vulvar Cancer, Wilms Tumor and Other Childhood Kidney Tumors.

In some embodiments of the compositions and methods of the present disclosure, a disease or disorder of the present disclosure includes, but is not limited to, a proliferative disease or disorder. In some embodiments, the proliferative disease or disorder is cancer. In some embodiments, the cancer involves the presence of a gene fusion that produces a chimeric RNA with sequences derived from two genes due to a deletion or translocation of DNA. Gene fusions pairs include but are not limited to: MAN2A1 and FER, DNAJB1 and PRKACA, BCR-ABL1, TMPRSS2 and ERG, EWSR1 and FLI1, PML and RARA, EML4 and ALK, KIAA1549 and BRAF, CCDC6 and RET, SS18 and SSX1, RUNX1 and RUNX1T1, PAX3 and FOXO1, NCOA4 and RET, ETV6 and RUNX1, FUS and DDIT3, SS18 and SSX2, NPM1 and ALK, KMT2A and AFF1, TCF3 and PBX1, STIL and TAL1, COL1A1 and PDGFB, CRTC1 and MAML2, NAB2 and STAT6, EWSR1 and ATF1, ETV6 and NTRK3, EWSR1 and ERG, EWSR1 and WT1, DNAJB1 and PRKACA, PAX7 and FOXO1, FUS and CREB3L2, CBFA2T3 and GLIS2, PAX8 and PPARG, KMT2A and MLLT1, EWSR1 and NR4A3, KMT2A and MLLT3, ASPSCR1 and TFE3, HMGA2 and LPP, JAZF1 and SUZ12, KIF5B and RET, FUS and ERG, SLC45A3 and ERG, NUP214 and ABL1, SET and NUP214, CD74 and ROS1, ETV6 and ABL1, TPM3 and NTRK1, PRKAR1A and RET, EWSR1 and CREB1, KMT2A and AFDN, EWSR1 and DDIT3, CLTC and ALK, ETV6 and PDGFRB, TPM3 and ALK, KMT2A and MLLT10, TMPRSS2 and ETV1, BRD4 and NUTM1, NUP98 and KDM5A, RANBP2 and ALK, CTNNB1 and PLAG1, KMT2A and ELL, TAF15 and NR4A3, FGFR3 and TACC3, PCM1 and JAK2, YWHAE and NUTM2B, STRN and ALK, CRTC3 and MAML2, CDH11 and USP6, CDKN2D and WDFY2, CIC and DUX4, SLC34A2 and ROS1, ATIC and ALK, CD74 and NRG1, MYB and NFIB, PRCC and TFE3, KIFSB and ALK, TMPRSS2 and ETV4, KMT2A and SEPT9, EWSR1 and POU5F1, FGFR1 and PLAG1, MN1 and ETV6, TBL1XR1 and TP63, KMT2A and EPS15, SLC45A3 and ELK4, DHH and RHEBL1, HEY1 and NCOA2, EZR and ROS1, GOPC and ROS1, HMGA2 and WIF1, KMT2A and CREBBP, SS18 and SSX4B, FAM131B and BRAF, EWSR1 and FEV, EWSR1 and PBX1, TPM4 and ALK, SND1 and BRAF, ACTB and GLI1, KMT2A and KNL1, KMT2A and SEPT6, SDC4 and ROS1, TFG and ALK, HNRNPA2B1 and ETV1, PTPRK and RSPO3, JAZF1 and PHF1, HMGA2 and RAD51B, KMT2A and MLLT11, TPR and NTRK1, AKAP9 and BRAF, FUS and CREB3L1, ETV6 and JAK2, HMGA2 and NFIB, KMT2A and AFF3, CHCHD7 and PLAG1, VTI1A and TCF71.2, LIFR and PLAG1, EWSR1 and ETV1, SRGAP3 and RAF1, KMT2A and AFF4, MEAF6 and PHF1, PAX3 and NCOA1, HAS2 and PLAG1, EWSR1 and NFATC2, HIP1 and ALK, GOLGA5 and RET, BCR and JAK2, EWSR1 and ETV4, DCTN1 and ALK, MBTD1 and CXorf67, NDRG1 and ERG, CARS and ALK, SFPQ and TFE3, KMT2A and ARHGAP26, KMT2A and EP300, KMT2A and TET1, PAX5 and JAK2, PPFIBP1 and ALK, YWHAE and NUTM2A, LRIG3 and ROS1, TFG and NTRK1, TPM3 and ROS1, SLC45A3 and ETV1, ERC1 and RET, SEC16A and NOTCH1, KTN1 and RET, SEC31A and JAK2, TCEA1 and PLAG1, QKI and NTRK2, RNF130 and BRAF, EIF3E and RSPO2, EWSR1 and ZNF444, LMNA and NTRK1, PPFIBP1 and ROS1, PWWP2A and ROS1, EWSR1 and YY1, FUS and ATF1, PAX3 and NCOA2, ZC3H7B and BCOR, BRD3 and NUTM1, CANT1 and ETV4, CIC and FOXO4, COL1A1 and USP6, EWSR1 and ZNF384, KMT2A and ABI1, KMT2A and ACTN4, KMT2A and CEP170B, KMT2A and FOXO3, KMT2A and GAS7, KMT2A and MLLT6, KMT2A and SEPT2, KMT2A and SEPT5, MSN and ALK, VCL and ALK, EZR and ERBB4, RELCH and RET, SLC3A2 and NRG1, TRIM24 and BRAF, KLC1 and ALK, ARID1A and MAST2, GPBP1L1 and MAST2, NFIX and MAST1, NOTCH1 and GABBR2, TADA2A and MAST1, ZNF700 and MAST1, TRIM24 and RET, TRIM33 and RET, SSBP2 and JAK2, KMT2A and EEFSEC, CLCN6 and BRAF, GNAIl and BRAF, MKRN1 and BRAF, NACC2 and NTRK2, FGFR1 and TACC1, TRIM27 and RET, HMGA2 and FHIT, HOOK3 and RET, PCM1 and RET, CEP89 and BRAF, CLIP1 and ROS1, ERC1 and ROS1, HLA and A and ROS1, LSM 14A and BRAF, MYOSA and ROS1, SHTN1 and ROS1, TP53 and NTRK1, TPM3 and ROS1, ZCCHC8 and ROS1, FGFR3 and BAIAP2L1, KLK2 and ETV1, ACSL3 and ETV1, NUP107 and LGR5, HMGA2 and CCNBIIP1, HMGA2 and COX6C, GATM and BRAF, HACL1 and RAF1, HERPUD1 and BRAF, ZSCAN30 and BRAF, SLC45A3 and BRAF, HMGA2 and LHFPL6, COL1A2 and PLAG1, ESRP1 and RAF1, IRF2BP2 and CDX1, TFG and NR4A3, CLTC and TFE3, EWSR1 and MYB, NONO and TFE3, FCHSD1 and BRAF, HMGA2 and EBF1, ACBD6 and RRP15, AGPATS and MCPH1, AGTRAP and BRAF, ARFIP1 and FHDC1, ATG4C and FBXO38, BBS9 and PKD1L1, CENPK and KMT2A, CNBP and USP6, DDX5 and ETV4, EIF3K and CYP39A1, EPC1 and PHF1, ERO1A and FERMT2, ETV6 and ITPR2, EWSR1 and NFATC1, EWSR1 and PATZ1, EWSR1 and SMARCA5, EWSR1 and SP3, FBXL18 and RNF216, FGFR1 and ZNF703, FN1 and ALK, FUS and FEV, GMDS and PDE8B, HMGA2 and ALDH2, IL6R and ATP8B2, INTS4 and GAB2, JPT1 and USHIG, KLK2 and ETV4, KMT2A and ABI2, KMT2A and ARHGEF12, KMT2A and BTBD18, KMT2A and CASP8AP2, KMT2A and CBL, KMT2A and CIP2A, KMT2A and CT45A2, KMT2A and DAB2IP, KMT2A and FOX04, KMT2A and FRYL, KMT2A and GMPS, KMT2A and GPHN, KMT2A and LASP1, KMT2A and LPP, KMT2A and MAPRE1, KMT2A and MYOIF, KMT2A and NCKIPSD, KMT2A and NRIP3, KMT2A and PDS5A, KMT2A and PICALM, KMT2A and PRRC1, KMT2A and SARNP, KMT2A and SH3GL1, KMT2A and SORBS2, KMT2A and TOP3A, KMT2A and ZFYVE19, MBOAT2 and PRKCE, MIA2 and GEMIN2, NF1 and AS1C2, NFIA and EHF, NTN1 and ACLY, OMD and USP6, PLA2R1 and RBMS1, PLXND1 and TMCC1, RAF1 and DAZL, RBM14 and PACS1, RGS22 and SYCP1, SEC31A and ALK, SEPT8 and AFF4, SLC22Al and CUTA, SLC26A6 and PRKAR2A, SLC45A3 and ETV5, SQSTM1 and ALK, SS18L1 and SSX1, SSH2 and SUZ12, SUSD1 and PTBP3, TCF12 and NR4A3, TECTA and TBCEL, THRAP3 and USP6, TMPRSS2 and ETV5, TPR and ALK, UBE2L3 and KRAS, WDCP and ALK, SS18 and USP6

In some embodiments of the methods of the present disclosure, a subject of the present disclosure has been diagnosed with the disease or disorder. In some embodiments, the subject of the present disclosure presents at least one sign or symptom of the disease or disorder. In some embodiments, the subject has a biomarker predictive of a risk of developing the disease or disorder. In some embodiments, the biomarker is a genetic mutation.

In some embodiments of the methods of the present disclosure, a subject of the present disclosure is female. In some embodiments of the methods of the present disclosure, a subject of the present disclosure is male. In some embodiments, a subject of the present disclosure has two XX or XY chromosomes. In some embodiments, a subject of the present disclosure has two XX or XY chromosomes and a third chromosome, either an X or a Y.

In some embodiments of the methods of the present disclosure, a subject of the present disclosure is a neonate, an infant, a child, an adult, a senior adult, or an elderly adult. In some embodiments of the methods of the present disclosure, a subject of the present disclosure is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days old. In some embodiments of the methods of the present disclosure, a subject of the present disclosure is at least 1, 2, 3, 4, 5, 6, 7, 8, 9, 10, 11 or 12 months old. In some embodiments of the methods of the present disclosure, a subject of the present disclosure is at least 1, 2, 3, 4, 5, 6, 7, 8, 9, 10, 15, 20, 25, 30, 35, 40, 45, 50, 55, 60, 65, 70, 75, 80, 85, 90, 95, 100 or any number of years or partial years in between of age.

In some embodiments of the methods of the present disclosure, a subject of the present disclosure is a mammal. In some embodiments, a subject of the present disclosure is a non-human mammal.

In some embodiments of the methods of the present disclosure, a subject of the present disclosure is a human.

In some embodiments of the methods of the present disclosure, a therapeutically effective amount comprises a single dose of a composition of the present disclosure. In some embodiments, a therapeutically effective amount comprises a therapeutically effective amount comprises at least one dose of a composition of the present disclosure. In some embodiments, a therapeutically effective amount comprises a therapeutically effective amount comprises one or more dose(s) of a composition of the present disclosure,

In some embodiments of the methods of the present disclosure, a therapeutically effective amount eliminates a sign or symptom of the disease or disorder. In some embodiments, a therapeutically effective amount reduces a severity of a sign or symptom of the disease or disorder.

In some embodiments of the methods of the present disclosure, a therapeutically effective amount eliminates the disease or disorder.

In some embodiments of the methods of the present disclosure, a therapeutically effective amount prevents an onset of a disease or disorder. In some embodiments, a therapeutically effective amount delays the onset of a disease or disorder. In some embodiments, a therapeutically effective amount reduces the severity of a sign or symptom of the disease or disorder. In some embodiments, a therapeutically effective amount improves a prognosis for the subject.

In some embodiments of the methods of the present disclosure, a composition of the present disclosure is administered to the subject systemically. In some embodiments, the composition of the present disclosure is administered to the subject by an intravenous route. In some embodiments, the composition of the present disclosure is administered to the subject by an injection or an infusion.

In some embodiments of the methods of the present disclosure, a composition of the present disclosure is administered to the subject locally. In some embodiments, the composition of the present disclosure is administered to the subject by an intraosseous, intraocular, intracerebrospinal or intraspinal route. In some embodiments, the composition of the present disclosure is administered directly to the cerebral spinal fluid of the central nervous system. In some embodiments, the composition of the present disclosure is administered directly to a tissue or fluid of the eye and does not have bioavailability outside of ocular structures. In some embodiments, the composition of the present disclosure is administered to the subject by an injection or an infusion,

In some embodiments, the compositions comprising the trans-splicing RNAs disclosed herein are formulated as pharmaceutical compositions. Briefly, pharmaceutical compositions for use as disclosed herein may comprise a fusion protein(s) or a polynucleotide encoding the fusion protein(s), optionally comprised in an AAV, which is optionally also immune orthogonal, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for oral, intravenous, topical, enteral, intraocular, and/or parenteral administration. In certain embodiments, the compositions of the present disclosure are formulated for intravenous administration.

### Nucleic Acids

Also provided herein are nucleic acid sequences encoding the trans-splicing nucleic acids disclosed herein for use in gene transfer and expression techniques described herein. It should be understood, although not always explicitly stated that the sequences provided herein can be used to provide the expression product as well as substantially identical sequences that produce a protein that has the same biological properties. These “biologically equivalent” or “biologically active” or “equivalent” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical nucleic acid sequence to the reference nucleic acid sequence when compared using sequence identity methods run under default conditions. Specific sequences are provided as examples of particular embodiments. Additionally, an equivalent polynucleotide is one that hybridizes under stringent conditions to the reference polynucleotide or its complement.

In some embodiments, the nucleic acid sequence encoding the trans-splicing nucleic acids comprise a DNA sequence comprising at least about 60%, about 65%, about 70%, about 75%, about 80%, about 85%, about 90%, about 95%, about 97.5%, about 98%, about 99%, or 100% sequence identity to any one of SEQ ID NO: 1-103. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 1. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 2. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 3. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 4. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 5. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 6. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 7. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 8. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 9. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 10. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 11. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 12. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 13. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 14. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 15. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 16. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 17. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 18. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 19. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 20. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 21. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 22. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 23. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 24. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 25. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 26. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 27. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 28. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 29. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 30. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 31. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 32. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 33. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 34. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 35. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 36. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 37. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 38. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 39. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 40. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 41. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 42. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 43. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 44. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 45. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 46. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 47. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 48. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 49. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 50. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 51. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 52. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 53. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 54. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 55. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 56. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 57. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 58. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 59. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 60. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 61. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 62. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 63. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 64. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 65. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 66. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 67. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 68. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 69. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 70. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 71. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 72. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 73. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 74. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 75. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 76. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 77. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 78. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 79. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 80. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 81. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 82. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 83. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 84. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 85. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 86. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 87. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 88. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 89. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 90. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 91. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 92. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 93. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 94. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 95. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 96. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 97. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 98. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 99. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 100. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 101. In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 102 . . . . In some embodiments, the nucleic acid sequence comprises at least about 60%, about 65%, about 70%, about 75%, about 80%, about 85%, about 90%, about 95%, about 97.5%, about 98%, about 99%, or 100% sequence identity with SEQ ID NO: 103.

The nucleic acid sequences (e.g., polynucleotide sequences) disclosed herein may be codon-optimized. Codon optimization refers to the fact that different cells differ in their usage of particular codons. This codon bias corresponds to a bias in the relative abundance of particular (RNAs in the cell type, By altering the codons in the sequence to match with the relative abundance of corresponding tRNAs, it is possible to increase expression. It is also possible to decrease expression by deliberately choosing codons for which the corresponding tRNAs are rare in a particular cell type. Codon usage tables may be used for mammalian cells, as well as for a variety of other organisms. Based on the genetic code, nucleic acid sequences coding for various Replacement Domains can be generated. In some embodiments, such a sequence is optimized for expression in a host or target cell, such as a host cell used to express the trans-splicing RNA containing a Replacement Domain in which the disclosed methods are practiced (such as in a mammalian cell, e.g., a human cell). Codon preferences and codon usage tables for a particular species can be used to engineer isolated nucleic acid molecules encoding a Replacement Domain (such as one encoding a protein having at least 80%, at least 85%, at least 90%, at least 91%, at least 92%, at least 93%, at least 94%, at least 95%, at least 96%, at least 97%, at least 98%, at least 99%, or 100% sequence identity to its corresponding wild-type protein) that takes advantage of the codon usage preferences of that particular species. For example, the Replacement Domains disclosed herein can be designed to have codons that are preferentially used by a particular organism of interest. In one example, a Replacement Domain nucleic acid sequence is optimized for expression in human cells, such as one having at least 70%, at least 80%, at least 85%, at least 90%, at least 91%, at least 92%, at least 93%, at least 94%, at least 95%, at least 96%, at least 97%, at least 98%, at least 99%, or 100% sequence identity to its corresponding wild-type or originating nucleic acid sequence. In some embodiments, an isolated trans-splicing nucleic acid molecule encoding at least one Replacement Domain (which can be part of a vector) includes at least one Replacement Domain coding sequence that is codon optimized for expression in a eukaryotic cell, or at least one Replacement Domain coding sequence codon optimized for expression in a human cell. In one embodiment, such a codon optimized Replacement Domain coding sequence has at least 80%, at least 85%, at least 90%, at least 91%, at least 92%, at least 93%, at least 94%, at least 95%, at least 96%, at least 97%, at least 98%, at least 99%, or 100% sequence identity to its corresponding wild-type or originating sequence. In another embodiment, a eukaryotic cell codon optimized nucleic acid sequence encodes a Replacement Domain having at least 80%, at least 85%, at least 90%, at least 91%, at least 92%, at least 93%, at least 94%, at least 95%, at least 96%, at least 97%, at least 98%, at least 99%, or 100% sequence identity to its corresponding wild-type or originating protein. In another embodiment, a variety of clones containing functionally equivalent nucleic acids may be routinely generated, such as nucleic acids which differ in sequence but which encode the same Replacement Domain protein sequence. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3.sup.rd Edition, W.H.5 Freeman and Co., NY).

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about Ix SSC to about 0.1×SSC;

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences described herein.

In some embodiments, the trans-splicing RNA further comprises a 5′ untranslated region. In some embodiments, the 5′ untranslated region increases the stability of the trans-splicing nucleic acid. In some embodiments, the 5′ untranslated region alters the localization of the trans-splicing nucleic acid. In some embodiments, the 5′ untranslated region alters the processing of the trans-splicing nucleic acid.

In some embodiments, the trans-splicing RNA further comprises a 3′ untranslated region. In some embodiments, the 3′ untranslated region increases the stability of the trans-splicing nucleic acid. In some embodiments, the 3′ untranslated region alters the localization of the trans-splicing nucleic acid. In some embodiments, the 3′ untranslated region alters the processing of the trans-splicing nucleic acid.

In some embodiments of the compositions of the present disclosure, the sequence encoding the trans-splicing RNA further comprises a sequence encoding a promoter capable of expressing the trans-splicing RNA in a eukaryotic cell.

### Vectors

The present disclosure provides vectors comprising or encoding nucleic acids as described herein. In some embodiments of the compositions and methods of the present disclosure, a vector comprises or encodes a trans-splicing nucleic acid of the present disclosure. In some embodiments, the vector encodes or comprises a DNA sequence. In some embodiments, the vector encodes or comprises an RNA sequence. In some embodiments, the vector comprises or encodes at least one trans-splicing nucleic acid of the present disclosure. In some embodiments, the vector comprises or encodes one or more trans-splicing nucleic acid(s) of the present disclosure. In some embodiments, the vector comprises or encodes two or more trans-splicing nucleic acids of the present disclosure.

In some embodiments of the compositions and methods of the present disclosure, a vector of the present disclosure is a viral vector. In some embodiments, the viral vector comprises a sequence isolated or derived from a retrovirus. In some embodiments, the viral vector comprises a sequence isolated or derived from a lentivirus. In some embodiments, the viral vector comprises a sequence isolated or derived from an adenovirus. In some embodiments, the viral vector comprises a sequence isolated or derived from an adeno-associated virus (AAV). In some embodiments, the viral vector is replication incompetent. In some embodiments, the viral vector is isolated or recombinant. In some embodiments, the viral vector is self-complementary.

In some embodiments of the compositions and methods of the present disclosure, the viral vector comprises a sequence isolated or derived from an adeno-associated virus (AAV). In some embodiments, the viral vector comprises an inverted terminal repeat sequence or a capsid sequence that is isolated or derived from an AAV of serotype AAV1, AAV2, AAV3, AAV4, AAV5, AAV6, AAV7, AAV8, AAV9, AAV10, AAV11 or AAV12. In some embodiments, the viral vector is replication incompetent. In some embodiments, the viral vector is isolated or recombinant (rAAV). In some embodiments, the viral vector is self-complementary (scAAV).

In some embodiments of the compositions and methods of the present disclosure, a vector of the present disclosure is a non-viral vector. In some embodiments, the vector comprises or consists of a nanoparticle, a micelle, a liposome or lipoplex, a polymersome, a polyplex or a dendrimer. In some embodiments, the vector is an expression vector or recombinant expression system. As used herein, the term “recombinant expression system” refers to a genetic construct for the expression of certain genetic material formed by recombination.

In some embodiments, the liposome, lipoplex, or nanoparticle can further comprise a non-cationic lipid, a PEG conjugated lipid, a sterol, or any combination thereof.

In some embodiments, the liposome, lipoplex, or nanoparticle further comprises a non-cationic lipid, wherein the non-ionic lipid is selected from the group consisting of distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O)-dimethyl PE), 18-1-trans PE, I-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine and non-cationic lipids described, for example, in WO2017/099823 or US2018/0028664.

In some embodiments, the liposome, lipoplex, or nanoparticle further comprises a conjugated lipid, wherein the conjugated lipid, wherein the conjugated-lipid is selected from the group consisting of PEG-diacylglycerol (DAG) (such as I-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di (tetradecanoyloxy) propyl-1-0-(w-methoxy (polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt.

In some embodiments, the liposome, lipoplex, or nanoparticle further comprises cholesterol or a cholesterol derivative.

In some embodiments, the liposome, lipoplex, or nanoparticle further comprises an ionizable lipid, a non-cationic lipid, a conjugated lipid that inhibits aggregation of particles, and a sterol. The amount of the ionizable lipid, the non-cationic lipid, the conjugated lipid that inhibits aggregation of particles, and the sterol can be varied independently. In some embodiments, the lipid nanoparticle comprises an ionizable lipid in an amount from about 20 mol % to about 90 mol % of the total lipid present in the particle, a non-cationic lipid in an amount from about 5 mol % to about 30 mol % of the total lipid present in the particle, a conjugated lipid that inhibits aggregation of particles in an amount from about 0.5 mol % to about 20 mol % of the total lipid present in the particle, and a sterol in an amount from about 20 mol % to about 50 mol % of the total lipid present in the particle.

The ratio of total lipid to DNA vector can be varied. For example, the total lipid to DNA vector (mass or weight) ratio can be from about 10:1 to about 30:1.

In some embodiments of the compositions and methods of the present disclosure, an expression vector, viral vector or non-viral vector provided herein, includes without limitation, an expression control element. An “expression control element” as used herein refers to any sequence that regulates the expression of a coding sequence, such as a gene. Examples of expression control elements include, but are not limited to, promoters, enhancers, microRNAs, post-transcriptional regulatory elements, polyadenylation signal sequences, and introns. Expression control elements may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. In some embodiments, expression control by a promoter is tissue-specific. Non-limiting examples of promoters include CMV, CBA, CAG, Cbh, EF-la, PGK, UBC, GUSB, UCOE, hAAT, TBG, Desmin, MCK, C5-12, NSE, Synapsin, PDGF, MecP2, CaMKII, mGluR2, NFL, NFH, nβ2, PPE, ENK, EAAT2, GFAP, MBP, and U6 promoters. An “enhancer” is a region of DNA that can be bound by activating proteins to increase the likelihood or frequency of transcription. Non-limiting examples of enhancers and posttranscriptional regulatory elements include the CMV enhancer and WPRE.

In some embodiments of the compositions and methods of the present disclosure, an expression vector, viral vector or non-viral vector provided herein, includes without limitation, an expression control element. An “expression control element” as used herein refers to any sequence that regulates the expression of a coding sequence, such as a gene. Examples of expression control elements include, but are not limited to, promoters, enhancers, microRNAs, post-transcriptional regulatory elements, polyadenylation signal sequences, 5′ or 3′ untranslated regions, and introns.

In some embodiments of the compositions and methods of the present disclosure, an expression vector, viral vector or non-viral vector provided herein, includes without limitation, vector elements such as an IRES or 2A peptide sites for configuration of “multicistronic” or “polycistronic” or “bicistronic” or tricistronic” constructs, i.e., having double or triple or multiple coding areas or exons, and as such will have the capability to express from mRNA two or more proteins from a single construct. Multicistronic vectors simultaneously express two or more separate proteins from the same mRNA. The two strategies most widely used for constructing multicistronic configurations are through the use of an IRES or a 2A self-cleaving site. An “IRES” refers to an internal ribosome entry site or portion thereof of viral, prokaryotic, or eukaryotic origin which are used within polycistronic vector constructs. In some embodiments, an IRES is an RNA element that allows for translation initiation in a cap-independent manner. The term “self-cleaving peptides” or “sequences encoding self-cleaving peptides” or “2A self-cleaving site” refer to linking sequences which are used within vector constructs to incorporate sites to promote ribosomal skipping and thus to generate two polypeptides from a single promoter, such self-cleaving peptides include without limitation, T2A, and P2A peptides or sequences encoding the self-cleaving peptides.

Expression control elements may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. In some embodiments, expression control by a promoter is tissue-specific, Non-limiting examples of promoters include CMV, CBA, CAG, Cbh, EF-1a, PGK, UBC, GUSB, UCOE, hAAT, TBG, Desmin, MCK, C5-12, NSE, Synapsin, PDGF, MecP2, CaMKII, mGluR2, NFL, NFH, nβ2, PPE, ENK, EAAT2, GFAP, MBP, H1 and U6 promoters. In some embodiments, the promoter is a sequence isolated or derived from a promoter capable of driving expression of a transfer RNA (tRNA). In some embodiments, the promoter is isolated or derived from an alanine tRNA promoter, an arginine tRNA promoter, an asparagine tRNA promoter, an aspartic acid tRNA promoter, a cysteine tRNA promoter, a glutamine tRNA promoter, a glutamic acid tRNA promoter, a glycine tRNA promoter, a histidine tRNA promoter, an isoleucine tRNA promoter, a leucine tRNA promoter, a lysine (RNA promoter, a methionine (RNA promoter, a phenylalanine tRNA promoter, a proline tRNA promoter, a serine tRNA promoter, a threonine tRNA promoter, a tryptophan tRNA promoter, a tyrosine tRNA promoter, or a valine tRNA promoter. In some embodiments, the promoter is isolated or derived from a valine tRNA promoter.

An “enhancer” is a region of DNA that can be bound by activating proteins to increase the likelihood or frequency of transcription. Non-limiting examples of enhancers and post-transcriptional regulatory elements include the CMV enhancer and WPRE.

In some embodiments, the vector is a viral vector. In some embodiments, the vector is an adenoviral vector, an adeno-associated viral (AAV) vector, or a lentiviral vector. In some embodiments, the vector is a retroviral vector, an adenoviral/retroviral chimera vector, a herpes simplex viral I or II vector, a parvoviral vector, a reticuloendotheliosis viral vector, a polioviral vector, a papillomaviral vector, a vaccinia viral vector, or any hybrid or chimeric vector incorporating favorable aspects of two or more viral vectors. In some embodiments, the vector further comprises one or more expression control elements operably linked to the polynucleotide. In some embodiments, the vector further comprises one or more selectable markers. In some embodiments, the AAV vector has low toxicity. In some embodiments, the AAV vector does not incorporate into the host genome, thereby having a low probability of causing insertional mutagenesis. In some embodiments, the AAV vector can encode a range of total polynucleotides from 0.3 kb to 4.75 kb. In some embodiments, examples of AAV vectors that may be used in any of the herein described compositions, systems, methods, and kits can include an AAV1 vector, a modified AAV1 vector, an AAV2 vector, a modified AAV2 vector, an AAV3 vector, a modified AAV3 vector, an AAV4 vector, a modified AAV4 vector, an AAV5 vector, a modified AAV5 vector, an AAV6 vector, a modified AAV6 vector, an AAV7 vector, a modified AAV7 vector, an AAV8 vector, an AAV9 vector, an AAV.rh10 vector, a modified AAV.rh10 vector, an AAV.rh32/33 vector, a modified AAV.rh32/33 vector, an AAV.rh43 vector, a modified AAV.rh43 vector, an AAV.rh74 vector, a modified AAV.rh74 vector, an AAV.rh64R1 vector, and a modified AAV.rh64RI vector and any combinations or equivalents thereof. In some embodiments, the lentiviral vector is an integrase-competent lentiviral vector (ICLV). In some embodiments, the lentiviral vector can refer to the transgene plasmid vector as well as the transgene plasmid vector in conjunction with related plasmids (e.g., a packaging plasmid, a rev expressing plasmid, an envelope plasmid) as well as a lentiviral-based particle capable of introducing exogenous nucleic acid into a cell through a viral or viral-like entry mechanism. In some embodiments, examples of lentiviral vectors that may be used in any of the herein described compositions, systems, methods, and kits can include a human immunodeficiency virus (HIV) I vector, a modified human immunodeficiency virus (HIV) 1 vector, a human immunodeficiency virus (HIV) 2 vector, a modified human immunodeficiency virus (HIV) 2 vector, a sooty mangabey simian immunodeficiency virus (SIVSM) vector, a modified sooty mangabey simian immunodeficiency virus (SIVSM) vector, a African green monkey simian immunodeficiency virus (SIVAGM) vector, a modified African green monkey simian immunodeficiency virus (SIVAGM) vector, an equine infectious anemia virus (EIAV) vector, a modified equine infections anemia virus (EIAV) vector, a feline immunodeficiency virus (FIV) vector, a modified feline immunodeficiency virus (FIV) vector, a Visna/maedi virus (VNV/VMV) vector, a modified Visna/maedi virus (VNV/VMV) vector, a caprine arthritis-encephalitis virus (CAEV) vector, a modified caprine arthritis-encephalitis virus (CAEV) vector, a bovine immunodeficiency virus (BIV), or a modified bovine immunodeficiency virus (BIV).

### Cells And Tissues

In some embodiments, the nucleic acids provided herein enable replacement of arbitrary, missing, or incorrect sequences in a target RNA molecule. The target RNA molecule may be in a cell, a tissue, an organ, or in an organism. The cell, tissue, or organ may be provided in vitro or in vivo. In some embodiments, DNA molecules provided herein enable replacement of arbitrary, missing, or incorrect sequences in RNA molecules of living cells. In some instances, the DNA molecule comprises an exonic or replacement sequence that can be trans-spliced into RNA in order to modify (e.g., fix) the sequence. In some instances, modification or fixing of the RNA via trans-splicing increases or decreases protein production. In some embodiments of the compositions and methods of the present disclosure, a cell of the present disclosure is a eukaryotic cell. In some embodiments, the cell is a mammalian cell. In some embodiments, the cell is a bovine, murine, feline, equine, porcine, canine, simian, or human cell. In some embodiments, the cell is a human cell. In some embodiments, the cell is a non-human mammalian cell such as a non-human primate cell. In some embodiments, a cell of the present disclosure is a somatic cell. In some embodiments, a cell of the present disclosure is a germline cell. In some embodiments, a germline cell of the present disclosure is not a human cell.

In some embodiments of the compositions and methods of the present disclosure, a cell of the present disclosure is a stem cell. In some embodiments, a cell of the present disclosure is an embryonic stem cell. In some embodiments, an embryonic stem cell of the present disclosure is not a human cell. In some embodiments, a cell of the present disclosure is a multipotent stem cell or a pluripotent stem cell. In some embodiments, a cell of the present disclosure is an adult stem cell. In some embodiments, a cell of the present disclosure is an induced pluripotent stem cell (iPSC). In some embodiments, a cell of the present disclosure is a hematopoietic stem cell (HSC).

In some embodiments of the compositions and methods of the present disclosure, an immune cell of the present disclosure is a lymphocyte. In some embodiments, an immune cell of the present disclosure is a T lymphocyte (also referred to herein as a T-cell). Examples of T-cells of the present disclosure include, but are not limited to, naïve T cells, effector T cells, helper T cells, memory T cells, regulatory T cells (Tregs) and Gamma delta T cells. In some embodiments, an immune cell of the present disclosure is a B lymphocyte. In some embodiments, an immune cell of the present disclosure is a natural killer cell. In some embodiments, an immune cell of the present disclosure is an antigen-presenting cell.

In some embodiments of the compositions and methods of the present disclosure, a muscle cell of the present disclosure is a myoblast or a myocyte. In some embodiments, a muscle cell of the present disclosure is a cardiac muscle cell, skeletal muscle cell or smooth muscle cell. In some embodiments, a muscle cell of the present disclosure is a striated cell.

In some embodiments of the compositions and methods of the present disclosure, a somatic cell of the present disclosure is an epithelial cell. In some embodiments, an epithelial cell of the present disclosure forms a squamous cell epithelium, a cuboidal cell epithelium, a columnar cell epithelium, a stratified cell epithelium, a pseudostratified columnar cell epithelium or a transitional cell epithelium. In some embodiments, an epithelial cell of the present disclosure forms a gland including, but not limited to, a pineal gland, a thymus gland, a pituitary gland, a thyroid gland, an adrenal gland, an apocrine gland, a holocrine gland, a merocrine gland, a serous gland, a mucous gland and a sebaceous gland. In some embodiments, an epithelial cell of the present disclosure contacts an outer surface of an organ including, but not limited to, a lung, a spleen, a stomach, a pancreas, a bladder, an intestine, a kidney, a gallbladder, a liver, a larynx or a pharynx. In some embodiments, an epithelial cell of the present disclosure contacts an outer surface of a blood vessel or a vein.

In some embodiments of the compositions and methods of the present disclosure, a brain cell of the present disclosure is a neuronal cell. In some embodiments, a neuron cell of the present disclosure is a neuron of the central nervous system. In some embodiments, a neuron cell of the present disclosure is a neuron of the brain or the spinal cord. In some embodiments, a neuron cell of the present disclosure is a neuron of a cranial nerve or an optic nerve. In some embodiments, a neuron cell of the present disclosure is a neuron of the peripheral nervous system. In some embodiments, a neuron cell of the present disclosure is a neuroglial or a glial cell. In some embodiments, a glial of the present disclosure is a glial cell of the central nervous system including, but not limited to, oligodendrocytes, astrocytes, ependymal cells, and microglia. In some embodiments, a glial of the present disclosure is a glial cell of the peripheral nervous system including, but not limited to, Schwann cells and satellite cells.

In some embodiments of the compositions and methods of the present disclosure, a liver cell of the present disclosure is a hepatocytes. In some embodiments, a liver cell of the present disclosure is a hepatic stellate cell. In some embodiments, a liver cell of the present disclosure is Kupffer cell. In some embodiments, a liver cell of the present disclosure is a sinusoidal endothelial cells,

In some embodiments of the compositions and methods of the present disclosure, a retinal cell of the present disclosure is a photoreceptor. In some embodiments, a photoreceptor cell of the present disclosure is a rod. In some embodiments, a retinal cell of the present disclosure is cone. In some embodiments, a retinal cell of the present disclosure is a bipolar cell. In some embodiments, a retinal cell of the present disclosure is a ganglion cell. In some embodiments, a retinal cell of the present disclosure is a horizontal cell. In some embodiments, a retinal cell of the present disclosure is an amacrine cell.

In some embodiments of the compositions and methods of the present disclosure, a heart cell of the present disclosure is a cardiomyocyte. In some embodiments, a heart cell of the present disclosure is a cardiac pacemaker cell.

In some embodiments of the compositions and methods of the present disclosure, a somatic cell of the present disclosure is a primary cell.

In some embodiments of the compositions and methods of the present disclosure, a somatic cell of the present disclosure is a cultured cell.

In some embodiments of the compositions and methods of the present disclosure, a somatic cell of the present disclosure is in vivo, in vitro, ex vivo or in situ.

In some embodiments of the compositions and methods of the present disclosure, a somatic cell of the present disclosure is autologous or allogeneic.

### Numbered Embodiments

Embodiment 1: A composition comprising a trans-splicing nucleic acid, comprising: (a) one or more Replacement Domains that encode a therapeutic sequence operably linked to; (b) one or more Intronic Domains that promote RNA splicing of the Replacement Domain comprising intronic trans-splicing enhancing sequence(s); (c) one or more Antisense Domains that promote binding to a target RNA molecule; and (c) one or more Stabilizing Domains that protect the trans-splicing nucleic acid from degradation by nucleases.

Embodiment 2: The composition of embodiment 1, wherein the Stabilizing Domains comprise sequences derived or isolated from the genome of a virus.

Embodiment 3: The composition of embodiment 1, wherein the Stabilizing Domains comprise sequences that form pseudoknots.

Embodiment 4: The composition of embodiment 1, wherein the Stabilizing Domains comprise sequences that form a triplex.

Embodiment 5: The composition of embodiment 1, wherein the Stabilizing Domains comprise sequences that promote nuclear localization of the trans-splicing nucleic acid.

Embodiment 6: The composition of embodiments 2, wherein the Stabilizing Domain is derived or isolated from a viral genome selected from the group consisting of: Kunjin virus, cell-fusing agent virus, tobacco etch virus, Montana myotis leukoencephalitis virus, Kaposi's sarcoma-associated herpesvirus, rhesus rhadinovirus, andequine herpesvirus 2, Apoi virus, Aroa virus, Bagaza virus, Banzi virus, Bouboui virus, Bukalasa bat virus, Cacipacore virus, Carey Island virus, Dakar bat virus, Cowbone Ridge virus, Dengue virus, Edge Hill virus, Entebbe bat virus, Gadgets Gully virus, Ilheus virus, Israel turkey meningoencephalomyelitis virus, Japanese encephalitis virus, Jugra virus, Jutiapa virus, Kadam virus, Kedougou virus, Kokobera virus, Koutango virus, Kyasanur Forest disease virus, Langat virus, Louping ill virus, Meaban virus, Modoc virus, Montana myotis leukoencephalitis virus, Murray Valley encephalitis virus, Ntaya virus, Omsk hemorrhagic fever virus, Phnom Penh bat virus, Powassan virus, Rio Bravo virus, Royal Farm virus, Saboya virus, Saint Louis encephalitis virus, Sal Vieja virus, San Perlita virus, Saumarez Reef virus, Sepik virus, Tembusu virus, Tick-borne encephalitis virus, Tyuleniy vírus, Uganda S virus, Wesselsbron virus, Usutu virus, West Nile virus, Yaounde virus, Yellow fever virus, Yokose virus, and Zika virus.

Embodiment 7: The composition of embodiments 3, wherein the Stabilizing Domain is derived or isolated from pseudoknot-forming sequence selected from the group consisting of: group 1 self-splicing introns from Azoarcus or Tetrahymena or Twort, drosophila sytl pre-mRNA, human CPEB3 ribozyme, E. coli RydC gene, prokaryotic plasmids I-complex or IncL/M or ColIB/P9, Mycobacterium bovis leuA mRNA, GlmS riboswitch ribozyme, Agrobacterium tumefa-ciens metA gene, L- and e-myc genes, Human interferon gamma mRNA, Ornithine decarboxylase antizyme, Prion mRNAs (human, cattle, yeast), Human and Tetrahymena telomerase, 16S rRNA, 16S rRNA, 18S V4 region, 23S rRNA, MI RNA component of bacterial RNase P, Neurospora VS ribozyme, Pyrimidine nucleotide synthase ribozyme, Alcohol dehydrogenase ribozyme (1-ribox02), a ribozyme, an aptamer, foot and mouse disease virus genome, Mengovirus genome, paraechovirus 1 genome, Aichivirus genome, hepatoviridae genomes, HCV, Classical swine fever virus genome, Bovine Viral Diarrhea virus genome, Porcine teschovirus, Cricket paralysis virus-like virus genomes, Giardia lamblia virus genome, Tobacco etch virus genome, retroviridae genomes, Nidovirales genomes, Totiviridae genomes, Luteoviridae genomes, Myoviridae genomes, Listeria monocytogenes phage genome, Murine leukemia virus genome, Hepatitis C virus genome, Influenza A and B genomes, Turnip yellow mosaic virus genomes, Tobacco mosaic virus-like virus genomes, Bamboo mosaic virus genome, Strawberry chlorotic fleck-associated virus genome; potato yellow vein virus genome, Tomato bushy stunt virus genome, Turnip crinkle virus genome, Encephalomyocarditis virus genome, Enterovirus genomes, Dengue virus genome, yellow fever virus genome, Japanese encephalitis virus genome, tick-borne encephalitis virus genome, Cauliflower mosaic virus genome, Barley yellow dwarf virus genome, Bacteriophage Qβ genome, Avian leukosis virus genome, Peach latent mosaic viroid genome, Large pospiviroidae genome, Sat C satellite RNA of Turnip crinkle virus genome, Hepatitis delta virus genome, and Marek's disease virus genome.

Embodiment 8: The composition of embodiments 4, wherein the Stabilizing Domain is derived or isolated from a sequences that forms triplexes selected from the group consisting of: MALAT1, NEAT1.

Embodiment 9: The composition of embodiments 5, wherein the Stabilizing Domain is derived or isolated from a gene that contains a sequence that promotes nuclear localization of the trans-splicing molecule and therefore protects the trans-splicing molecule from cytoplasmic RNA nucleases.

Embodiment 10: The composition of embodiments 1-9, wherein the Replacement Domain is derived or isolated from a human gene selected from the group consisting of: GLB1 (GM1 gangliosidosis); GBA (Gaucher disease); GM2A (GM2 gangliosidosis); PCSK9, LDLR, APOB, APOE (Familial hypercholesterolemia); GAA (Pompe disease); MYOC, OPTN, TBK1, WDR36, CYPIB1 (Open Angle Glaucoma); IDS (Hunter syndrome or Mucopolysaccharidosis 2); IDUA (Hurler syndrome or Mucopolysaccharidosis 1); CLN3 (Batten disease); F9 (Hemophilia B); F8 (Hemophilia A), LAMP2 (Danon disease); GLA (Fabry disease); SLC2A1 (glucose transporter deficiency type 1); UBE3A (Angelman syndrome); MYOC, OPTN, TBK1, WDR36, CYPIB1 (Open Angle Glaucoma); IDUA (Hurler syndrome or Mucopolysaccharidosis 1); IDS (Hunter syndrome or Mucopolysaccharidosis 2); CLN3 (Batten disease); LMNA (Limb-girdle muscular dystrophy type 1B); DMD (Duchenne muscular dystrophy); DYSF (Limb-girdle muscular dystrophy type 2B); SGCB (Limb-girdle muscular dystrophy type 2E); SGCG (Limb-girdle muscular dystrophy type 2C); SGCA (Limb-girdle muscular dystrophy type 2D); SOCD (Limb-girdle muscular dystrophy type 2F); DUX4, D4Z4 (Facioscapulohumeral muscular dystrophy); USHA2A, RPGR, RP2, RHO, PRPF31, USH1F, PRPF3, PRPF6 (Retinitis pigmentosa).

Embodiment 11: The composition of embodiments 1-9, wherein the Replacement Domain is derived or isolated from an expression-enhancing sequence selected from the group consisting of: Woodchuck Hepatitis Virus (WHV) Post-transcriptional Regulatory Element (WPRE), triplex from MALAT1, the PRE of Hepatitis B virus (HPRE), and an iron response element.

Embodiment 12: The composition of any one of embodiments 1-9, wherein the Antisense Domain is complementary to sequences derived or isolated from a human gene selected from the group consisting of: TNFRSF13B (common variable immune deficiency), ADA, CECR1 (Adenosine deaminase deficiency), IL2RG (X-linked severe combined immunodeficiency), HBB (Beta-thassalemia), HBA1, HBA2 (alpha-thassalemia), U2AF1 (myelodysplastic syndrome), SOD1, TARDBP, FUS, MATR3, SOD1, C9ORF72 (Amyotrophic lateral sclerosis), MAPT, PORN (Frontotemporal dementia with parkinsonism), CDH23, MYO7A, USH2A (Usher's syndrome), GALC (Krabbe disease), SMPD1, NPC1, NPC2 (Niemann Pick disease), PRNP (prion disease), SCN1A (Dravet syndrome), PINK1, ATPGAP2 (early-onset Parkinson's disease), ATXN1, ATXN2, ATXN3, PLEKHG4, SPTBN2, CACNA1A, ATXN7, TTBK2, PPP2R2B, KCNC3, PRKCG, ITRP1, TBP, KCND1, FGF14 (spinocerebellar ataxias), SCN1A, SCN2A, CACNA1A, GRIN2B, GRIN2A, MECP2, FOXG1, SLC6A1, PRRT2, PTEN, KCNQ2, KCNQ3, STARD7, CLRN1 (genetic epilepsy disorders), ATM (Ataxia-telangiectasia), GLB1 (GM1 gangliosidosis), GBA (Gaucher disease), GM2A (GM2 gangliosidosis), UBE3A (Angelman syndrome), SLC2A1 (glucose transporter deficiency type 1), LAMP2 (Danon disease), GLA (Fabry disease), PKD1, PKD2 (Autosomal dominant polycystic kidney disease), GAA (Pompe disease), PCSK9, LDLR, APOB, APOE (Familial hypercholesterolemia), MYOC, OPTN, TBK1, WDR36, CYPIB1 (Open Angle Glaucoma), IDUA (Hurler syndrome or Mucopolysaccharidosis 1), IDS (Hunter syndrome or Mucopolysaccharidosis 2), CLN3 (Batten disease), DMD (Duchenne muscular dystrophy), LMNA (Limb-girdle muscular dystrophy type 1B), DYSF (Limb-girdle muscular dystrophy type 2B), SGCA (Limb-girdle muscular dystrophy type 2D), SGCB (Limb-girdle muscular dystrophy type 2E), SGCG (Limb-girdle muscular dystrophy type 2C), SGCD (Limb-girdle muscular dystrophy type 2F), DUX4, D4Z4 (Facioscapulohumeral muscular dystrophy), F9 (Hemophilia B), F8 (Hemophilia A), USHA2A, RPGR, RP2, RHO, PRPF31, USH1F, PRPF3, PRPF6 (Retinitis pigmentosa), CFTR (cystic fibrosis), GJB2, GJB6, STRC, DFNA1, DENA14 (autosomal dominant hearing impairment), POU3F3 (nonsyndromic hearing loss)

Embodiment 13: The composition of any one of embodiments 1-12, wherein the trans-splicing RNA comprises an untranslated region that alters the localization, processing, or transport of the trans-splicing nucleic acid.

Embodiment 14: the composition of any one of embodiments 1-13, wherein the sequence comprising the trans-splicing nucleic acid comprises a sequence that is bound by an RNA-binding protein that increases the trans-splicing efficiency.

Embodiment 15: the composition of any of one embodiments 1-14, wherein the trans-splicing nucleic acid is RNA, DNA, a DNA/RNA hybrid, nucleic acid analog, a chemically-modified nucleic acid, or a chimera composed of two or more nucleic acids or nucleic acid analogs.

Embodiment 16: the composition of any of one embodiments 1-15, wherein the wherein the trans-splicing nucleic acid molecule further comprises a heterologous promoter.

Embodiment 17: the composition of any of one embodiments 16, wherein the promoter is isolated or derived from a promoter capable of driving expression of a transfer RNA (tRNA).

### EXAMPLES

The following examples are included for illustrative purposes only and are not intended to limit the scope of the invention.

### Example 1: The Effect of Stabilizing Sequences on Trans-Splicing Stability

The reporter and trans-splicing molecules described in FIG. 3 were encoded in DNA plasmids and transfected into HEK293T cells in biological triplicate along with a plasmid encoding mCherry fluorescent protein driven by a pGK promoter as a transfection control. 48 hours later, cellular fluorescence in the GFP and mCherry channels was measured by FACS analysis. The mean GFP signal among of each replicate was normalized to the mean mCherry signal and reported in the table.

Trans-splicing molecules containing sequences that block the activity of cellular nucleases were transiently-transfected in HEK293T cells and RNA harvested in order to assess whether the presence of putative stabilizing sequences resulted in increased cellular levels of the trans-splicing molecules. RNA was subjected to reverse transcription and quantitative PCR using primers that amplify the trans-splicing molecule and a housekeeping gene. Indeed, stabilizing sequences that increased trans-splicing activity also increased the levels of the trans-splicing molecule.

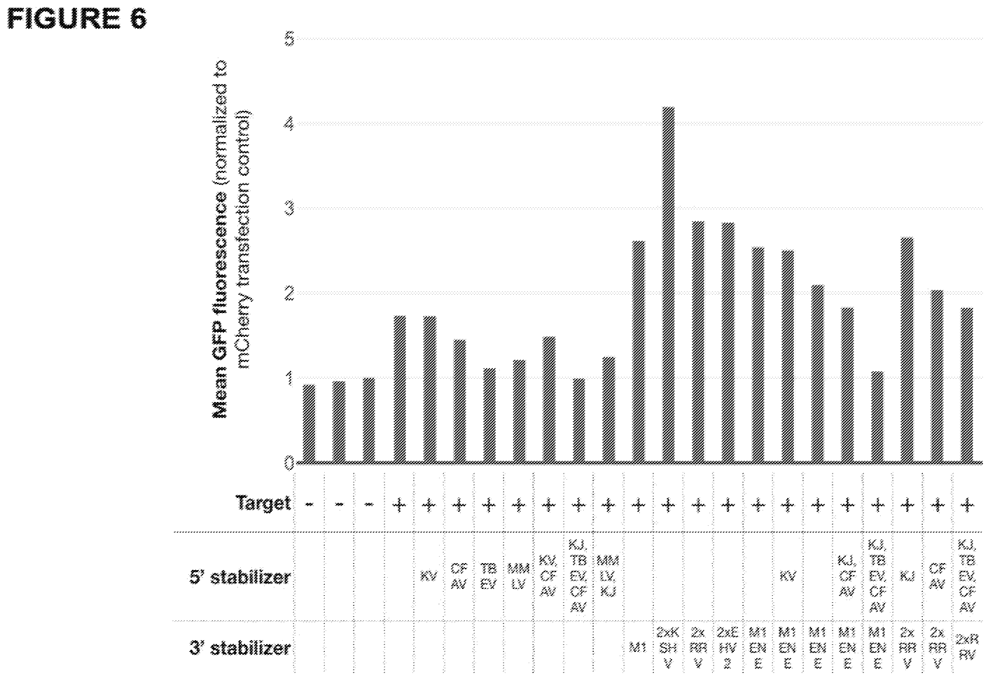

Results are depicted in FIG. 6. Each bar represents a separate trans-splicing molecule with a distinct set of stabilizing sequences. “+” means that the trans-splicing molecule carries a binding domain antisense to the reporter and therefore is capable of generating GFP signal upon successful trans-splicing. “-” indicates non-targeting trans-splicing molecules that carry a scrambled binding domain and cannot target the reporter. Stabilizing Domains were appended to the 3′ or 5′ end of each trans-splicing molecule. The abbreviations for each stabilizing sequences are as follows: “KV” means Kunjin virus exonuclease resistant RNA (“xrRNA”), “CFAV” means cell-fusing agent virus xrRNA, “TBEV” means tobacco etch virus xrRNA, “MMLV” means Montana myotis leukoencephalitis virus xrRNA, “M1” means human MALAT1 triplex, “2×KSHV” means a pair of concatenated Kaposi's sarcoma-associated herpesvirus expression and nuclear retention elements (ENEs), “2×RRV” means a pair of concatenated rhesus rhadinovirus ENEs, “2×EHV2” means a pair of concatenated equine herpesvirus 2 ENEs, “M1 ENE” means the human MALAT1 ENE. The data indicates that the addition of stabilizing sequences increased the efficiency of trans-splicing up to 4-fold compared to non-targeting trans-splicing molecules while a trans-splicing molecule carrying no stabilizing sequences (fourth bar from left) increased trans-splicing 1.8-fold compared to non-targeting trans-splicing molecules.

### Example 2: Stabilizing Sequences for 5′ Terminal Trans-Splicing

A split GFP reporter that carries a C-terminal portion of GFP (“C-GFP”) but lacks an N-terminal GFP sequence required for fluorescence is designed to assess the stability of sequences in stabilizing 5′ terminal trans-splicing (FIG. 4A). In the reporter, this N-terminal GFP sequence is replaced by a short exon with a stop codon that is flanked by introns. The N-terminal sequence (“N-GFP”) is the replacement sequence within an RNA trans-splicing molecule that is flanked by one intronic sequence, one antisense sequence, and one or more terminal stabilizing sequences (“3′-end stabilized trans-splicing RNA” and “5′-end stabilized trans-splicing RNA”).

The reporter and stabilizing sequences are encoded in DNA plasmids and transfected into HEK293T cells in biological triplicate along with a plasmid encoding fluorescent protein driven by a pGK promoter as a transfection control. 48 hours later, cellular fluorescence in the GFP and mCherry channels is measured by FACS analysis. The mean GFP signal among of each replicate is normalized to the mean mCherry signal and reported in the table.

FIG. 4B illustrates the activity of the reporter alone so that cis-splicing produces a GFP sequence interrupted by a stop codon therefore producing no GFP signal. FIG. 4C illustrates the activity of the reporter in the presence of the trans-splicing molecule without inclusion of stabilizing sequences in the trans-splicing molecule so that similarly cis-splicing occurs primarily and GFP signal is not efficiently produced. FIG. 4D illustrates the activity of the reporter in the presence of the trans-splicing molecule with inclusion of stabilizing sequences so that trans-splicing occurs primarily and GFP signal is efficiently produced.

### Example 3: Stabilizing Sequences for 3′ Terminal Trans-Splicing

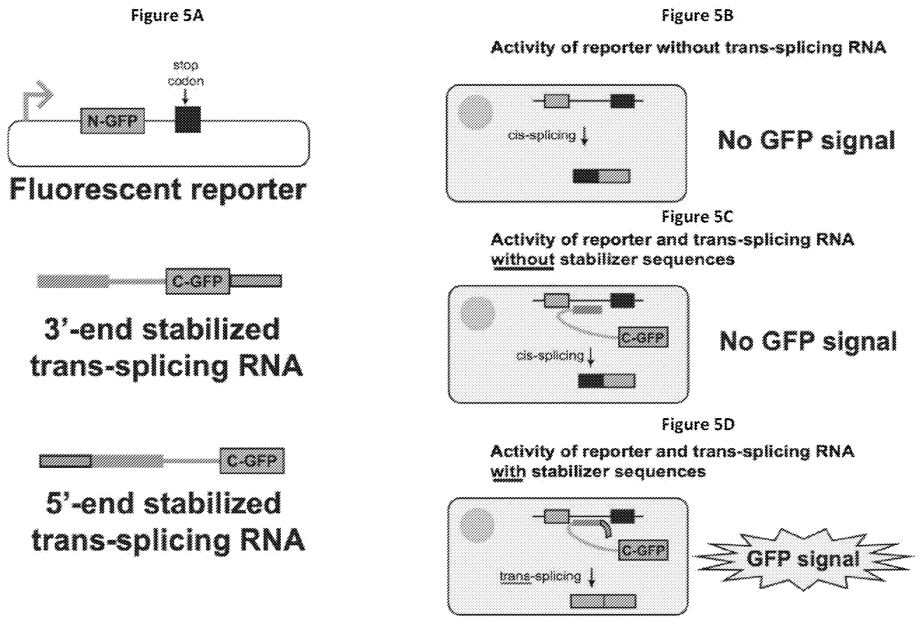

A split GFP reporter that carries a N-terminal portion of GFP (“N-GFP”) but lacks an C-terminal GFP sequence required for fluorescence (FIG. 5A), In the reporter, this C-terminal GFP sequence is replaced by a short exon with a stop codon that is flanked by introns. The C-terminal sequence (“C-GFP”) is the replacement sequence within an RNA trans-splicing molecule that is flanked by one intronic sequence, one antisense sequence, and one or more terminal stabilizing sequences,

The reporter and stabilizing sequences are encoded in DNA plasmids and transfected into HEK293T cells in biological triplicate along with a plasmid encoding fluorescent protein driven by a pGK promoter as a transfection control. 48 hours later, cellular fluorescence in the GFP and mCherry channels is measured by FACS analysis. The mean GFP signal among of each replicate is normalized to the mean mCherry signal and reported in the table.

FIG. 5B illustrates the activity of the reporter alone so that cis-splicing produces a GFP sequence interrupted by a stop codon therefore producing no GFP signal. FIGURE SC illustrates the activity of the reporter in the presence of the trans-splicing molecule without inclusion stabilizing sequences in the trans-splicing molecule so that similarly cis-splicing occurs primarily and GFP signal is not efficiently produced. FIG. 5D illustrates the activity of the reporter in the presence of the trans-splicing molecule with inclusion of stabilizing sequences so that trans-splicing occurs primarily and GFP signal is produced.

### Example 4: Stabilizing Sequences for 3′ Terminal Trans-Splicing

In some instances, experiments are conducted with either transiently-transfected reporter and trans-splicing molecule or systems packaged in lentivirus.

In some instances, an RNA trans-splicing system carrying various stabilizing sequences such as, a Woodchuck Hepatitis Virus (WHV) post-transcriptional Regulatory Element (WPRE) to assess the ability of an RNA trans-splicing system containing stabilizing sequences to increase protein production from specific mRNAs is synthesized. In other instances, a reporter that contains a firefly luciferase coding sequence and the last 2 exons and intervening intron of MBNL1 is synthesized. This assay is qualitative but is useful because it is what end-users in cell biology often use when attempting to answer scientific questions about the presence, absence, or general magnitude of a transcript. This reporter is based on the pMIR-GLO luciferase vector that is used to assess the stability and protein production from a model miRNA.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. (canceled)

2. A composition comprising a trans-splicing ribonucleic acid (RNA), comprising: (a) one or more replacement domains that encode a therapeutic sequence operably linked to; (b) one or more intronic domains that promote RNA splicing of the replacement domain; (c) one or more antisense domains that promote binding to a target RNA molecule; and (d) one or more stabilization domains reduce the susceptibility of the trans-splicing RNA to nucleases as compared to a trans-splicing RNA without one or more stabilization domains.

3-94. (canceled)


SEQUENCE LISTING (103 sequences)

SEQ ID NO: 1 - DNA, 81 bp, synthetic construct
  source 1..81: mol_type = other DNA
ttagtgaggatgtcagaccacggccatggcgtgccactctgcggagagtgcagtctgcgacagtgccccaggaggactggg

SEQ ID NO: 2 - DNA, 67 bp, synthetic construct
  source 1..67: mol_type = other DNA
acaggagcagggcatgaaaatgtcgggcatgacgaacccgctcccccgagtcccctggcaacagggt

SEQ ID NO: 3 - DNA, 231 bp, synthetic construct
  source 1..231: mol_type = other DNA
cacagatcatggaatgatgcggcagcgcgcgagagcaacggggaagtggtggcacccgacgcaccatccatgaagcaatacttcgtgagaccccccctgaccagcaaagggggcagaccggtcaggggtgaggaatgcccccagagtgcattacggcagcacgccagtgagagtggcgacgggaaaatggtcgatcccgacgtagggcactctgaaaaattttgtgagacc

SEQ ID NO: 4 - DNA, 170 bp, synthetic construct
  source 1..170: mol_type = other DNA
tggaaaaaatgcgagtgagggcaactctgggattagctcaatgggtgtgacgaccctacccttccgcatttgtaaataattgagccagtcatttccgtagggaagagagttattcgctcctctcgagattgagcggcctgctccttggagcatgagatgggaggcccgaa

SEQ ID NO: 5 - DNA, 78 bp, synthetic construct
  source 1..78: mol_type = other DNA
gaaaggcaaggtacggattagccgtaggggcttgagaaccccccctccccactcattttatttcctctatgaggaagg

SEQ ID NO: 6 - DNA, 75 bp, synthetic construct
  source 1..75: mol_type = other DNA
tttgggcaaggtgcaggttagctgcaggggcttgaaaaacccccccccccattcaagacttttagtgcattagtt

SEQ ID NO: 7 - DNA, 80 bp, synthetic construct
  source 1..80: mol_type = other DNA
accccgtaaggaaggacaaggctgtccttgagtactaacgacactccggccccagttcccagagccagggttttagctcc

SEQ ID NO: 8 - DNA, 80 bp, synthetic construct
  source 1..80: mol_type = other DNA
cgcgcgcaaggaaggacatggctgtccttgggtacgaacgacaccccgcccccagttctcaaggttagagttataacctc

SEQ ID NO: 9 - DNA, 74 bp, synthetic construct
  source 1..74: mol_type = other DNA
ccatcgcaagggaggattttcctcgggtactgaccataccccgaccccagtccgataggtcatggaatgacccc

SEQ ID NO: 10 - DNA, 77 bp, synthetic construct
  source 1..77: mol_type = other DNA
ctcccgtaaggaaagcgcaagctttgagcattgacaacgctccggccccagtcccccaggttatgggagaataaccc

SEQ ID NO: 11 - DNA, 86 bp, synthetic construct
  source 1..86: mol_type = other DNA
ctagcgcaaggaaggaaagtcgcagactaccttgggtgttgacgacactccgcccccagtcaccttggccgaaggttaaacggcat

SEQ ID NO: 12 - DNA, 259 bp, synthetic construct
  source 1..259: mol_type = other DNA
ccacgcaatgggaggcatcatttcgcctccgggtgctgactactccccgtccccgtccctaggtcaagtgaatgaccccgtgatgttgtgatgacatcataccaggcttggcatcctggcaactgccacccgcaaggggagggttttctaactctccgggtgttgacgacaccccggccccagtccccaaggtcttgggaaaaagaccccgaagtgttgcaaggacactaatcaccgaaaggtgagggcgcacaggatc

SEQ ID NO: 13 - DNA, 84 bp, synthetic construct
  source 1..84: mol_type = other DNA
cacccgcaaggggagggttttctaactctccgggtgttgacgacaccccggccccagtccccaaggtcttgggaaaaagacccc

SEQ ID NO: 14 - DNA, 87 bp, synthetic construct
  source 1..87: mol_type = other DNA
tcagcgcaaggaagggaagcttgaggctaccttaggtggtgacgacacctcgcccccagtctcccgggttggggataatacaacctc

SEQ ID NO: 15 - DNA, 83 bp, synthetic construct
  source 1..83: mol_type = other DNA
cccccgcaaggaggggtgtgtgtttcacgcccctgggagttaacgattctccggccccagttcctaggtccagggagggcccc

SEQ ID NO: 16 - DNA, 85 bp, synthetic construct
  source 1..85: mol_type = other DNA
cccgcgcaaggaaggacacgtaaatcacgtgttcttgggagttgacgactctccgcccccagtccccaggtcagggtatgactcc

SEQ ID NO: 17 - DNA, 83 bp, synthetic construct
  source 1..83: mol_type = other DNA
ccaccgcaagggagagggatcccctctcgggtttggacgacaccccggccccagtcccccaggtcatgggaaaaactgacccc

SEQ ID NO: 18 - DNA, 87 bp, synthetic construct
  source 1..87: mol_type = other DNA
tcagcgcaaggaaggaaagctggacgctaccttaggtggtgacgacacctcgcccccagtctcccaggttggggatcgtacaacttc

SEQ ID NO: 19 - DNA, 83 bp, synthetic construct
  source 1..83: mol_type = other DNA
cccccgcaaggagggacgtgtgcatcacgtttctgggagttaacggctctccggccccagttcctaggtccaggtaggatccc

SEQ ID NO: 20 - DNA, 87 bp, synthetic construct
  source 1..87: mol_type = other DNA
cccgcgcaaggaaggatgcgatgaaaacgcttgtccttgggagttgacgactctccgcccccagtccccaggtcagggtatgacccc

SEQ ID NO: 21 - DNA, 83 bp, synthetic construct
  source 1..83: mol_type = other DNA
ccaccgcaagggagagggattccctctcgggtgtggacgacaccccggccccagtcccctaggtcatgggaaaaactgacccc

SEQ ID NO: 22 - DNA, 86 bp, synthetic construct
  source 1..86: mol_type = other DNA
ctagcgcaaggaaggaaagtcgcagactaccttgggtgttgacgacactccgcccccagtcaccttggccaaaggttaaatggcat

SEQ ID NO: 23 - DNA, 81 bp, synthetic construct
  source 1..81: mol_type = other DNA
cccacgcaaggggaggcatcatattgcctccgggtgctgacgacaccccgtccccagtccctaggtcaagtgaatgacccc

SEQ ID NO: 24 - DNA, 84 bp, synthetic construct
  source 1..84: mol_type = other DNA
cacccgcaaggggggagttttctaactccccgggtgttgacgacaccccggccccagtccccaaggtcttgggaaaaagacccc

SEQ ID NO: 25 - DNA, 74 bp, synthetic construct
  source 1..74: mol_type = other DNA
acaggagcagggcatgaaaatgtcgggcatgacgaacccgctcccccgagtcccctggcaacagggtgtgttcc

SEQ ID NO: 26 - DNA, 75 bp, synthetic construct
  source 1..75: mol_type = other DNA
ttcgagcagggcacattagtgtcgggcgtgacgcacccgctcccctcagtcccctgtgcaacagggagggcactt

SEQ ID NO: 27 - DNA, 77 bp, synthetic construct
  source 1..77: mol_type = other DNA
acggggcaacagggagaaatcccggggtagcgaacctcctccgttaatgtgaaaaagtatggggaaagaactcatct

SEQ ID NO: 28 - DNA, 78 bp, synthetic construct
  source 1..78: mol_type = other DNA
tcagagcagggcacaatagtgtcgggcctgacgaccccgctcccccgagtcgcccaacggagtttggctcaactctaa

SEQ ID NO: 29 - DNA, 78 bp, synthetic construct
  source 1..78: mol_type = other DNA
gcagcgcagggcatgaaaatgtcgggcctgacgaacccgcgcacccgagtcccccagttggggaagggatccttgcat

SEQ ID NO: 30 - DNA, 79 bp, synthetic construct
  source 1..79: mol_type = other DNA
gcggagcaacagggagaaatcccggggaattgcgaaccccctccgaaatgtgaaaaattatggggaaaagtacccatct

SEQ ID NO: 31 - DNA, 75 bp, synthetic construct
  source 1..75: mol_type = other DNA
gagcagggcacaacagtgtcgggcctgacgaccccgctcccccgagtcaccaactggagtttggctcaactccaa

SEQ ID NO: 32 - DNA, 86 bp, synthetic construct
  source 1..86: mol_type = other DNA
ggaaggcaacacaccagtaatctggtggggtgagttgcgacacccccttgtgagcaaccgacctagccggtctattgaccggcttg

SEQ ID NO: 33 - DNA, 93 bp, synthetic construct
  source 1..93: mol_type = other DNA
acagggcaacagggatcgccaacatcagatcccgggtgagtgacgacaccccccatgtgaatcgtcaacttaggaacacattcaaatagagga

SEQ ID NO: 34 - DNA, 85 bp, synthetic construct
  source 1..85: mol_type = other DNA
acagggcaacagggatcaccaacatcagatcccgggtgagtgacgacaccccccatgtgaatcgtcgatacaaaaacacgatagg

SEQ ID NO: 35 - DNA, 78 bp, synthetic construct
  source 1..78: mol_type = other DNA
acaccgcaaggaagagaaatcttgggtggtaacaacaccccggccccagttctcgcgtgccacgagtcattggcacaa

SEQ ID NO: 36 - DNA, 79 bp, synthetic construct
  source 1..79: mol_type = other DNA
aaaccgcaaggaaggagcaatccttgggtattaacgacaccccggccccagttcccgaagtcaaggggacccttgaccc

SEQ ID NO: 37 - DNA, 81 bp, synthetic construct
  source 1..81: mol_type = other DNA
cattgcagcagagatttatctcgggggagttacgcccctccattgccagtagagtttgcatgtctctataaacatgacgtt

SEQ ID NO: 38 - DNA, 82 bp, synthetic construct
  source 1..82: mol_type = other DNA
aatatggcagcagagcttgtctcggggattcacgctccccccattgtgagtgtgtcgaactggtttcgaaggacgtctagaa

SEQ ID NO: 39 - DNA, 89 bp, synthetic construct
  source 1..89: mol_type = other DNA
atccggcaacagagagtttatgtctcggggcctcacgcaccccccgttgtgagtgaagtcctttctggccatttagtggtcaggaaggg

SEQ ID NO: 40 - DNA, 91 bp, synthetic construct
  source 1..91: mol_type = other DNA
gcagggcaacaaagttctaacgaactagggtgagtagcgtcaccccccggttgtgaaaacgattgcgactagaactaaagtcgagagtctc

SEQ ID NO: 41 - DNA, 81 bp, synthetic construct
  source 1..81: mol_type = other DNA
ttagagcagggcacgaaagtgtcgggcatgacgcacccgctcccccgagtcccctgaaaatagggtgggcaatgcactcct

SEQ ID NO: 42 - DNA, 87 bp, synthetic construct
  source 1..87: mol_type = other DNA
tttgagcagggcacgaaagtgtcgggcctgacgcacccgctcccccgagtcccctggaaacagggtgggcctcgaaaaatccaccgt

SEQ ID NO: 43 - DNA, 76 bp, synthetic construct
  source 1..76: mol_type = other DNA
gcagggcaacaggaagaaattccgggtgattagccacaccccccgaaacgtgatttatatgatgacaagaatcaga

SEQ ID NO: 44 - DNA, 78 bp, synthetic construct
  source 1..78: mol_type = other DNA
tgggggcagccgggggaaaccctggggcttggcgacctccccccacaagccatcatgcgaaattaaggcagccgcgag

SEQ ID NO: 45 - DNA, 96 bp, synthetic construct
  source 1..96: mol_type = other DNA
tgggggcagccgggagtcaaactcccggggcctggcgaccccccccttccgcctccaaaaattaaggcagccccgagggagctctcctcggtgtga

SEQ ID NO: 46 - DNA, 64 bp, synthetic construct
  source 1..64: mol_type = other DNA
caagggcaggttgaaaggggcttggcgacccccccctaaccagccacgcgcactgtgcgtgcgc

SEQ ID NO: 47 - DNA, 62 bp, synthetic construct
  source 1..62: mol_type = other DNA
ccggggcaggttgaaaggggtttcacgcacccccccctaccagtgccgtcgttggtaacgcg

SEQ ID NO: 48 - DNA, 65 bp, synthetic construct
  source 1..65: mol_type = other DNA
caagggcaggttgaaaggggcttggcgacccccccctaactagccacgcgcactgatgtgcgcgg

SEQ ID NO: 49 - DNA, 62 bp, synthetic construct
  source 1..62: mol_type = other DNA
ccggggcaggttgaaaggggcatcacgcacccccccctaccagtgccgtcgttggtaacgcg

SEQ ID NO: 50 - DNA, 79 bp, synthetic construct
  source 1..79: mol_type = other DNA
acgggcaaggagcgcaagctggggctttccgacccccccccccaggacgattccccgcttggtaaaaagggccaggcca

SEQ ID NO: 51 - DNA, 77 bp, synthetic construct
  source 1..77: mol_type = other DNA
ctccaggcagcagcagacgcaagtctgggggaaacgatcgctcctccctctgcagatctctagctcggatagagcgg

SEQ ID NO: 52 - DNA, 89 bp, synthetic construct
  source 1..89: mol_type = other DNA
atgtggcaaggggcctgctaacacaggccggggcttcctgacccccccaccccaaggctgttccccgctcggtaaaaagggccgggcca

SEQ ID NO: 53 - DNA, 86 bp, synthetic construct
  source 1..86: mol_type = other DNA
atgaggcaaggagcctgctaacacaggctggggcttcctgaccccccctccccaaggcggttccccgctcggtaaaaagggccggg

SEQ ID NO: 54 - DNA, 84 bp, synthetic construct
  source 1..84: mol_type = other DNA
gtgaggcaaggagacattccaggaatgtctggggctttccgaccccccctccccaggacggttccccgctgagtaaaaagggct

SEQ ID NO: 55 - DNA, 91 bp, synthetic construct
  source 1..91: mol_type = other DNA
aatgtggcaaggggcctgtccaagacaggccggggctttccgaccccccacccccaggacggttccccgctcggtaaaaagggccgggcta

SEQ ID NO: 56 - DNA, 93 bp, synthetic construct
  source 1..93: mol_type = other DNA
ttgcggcaaggtcggccgactgatcatcggctgaggaggttcccgccctccccgccccaggggtctccccgctgggtaaaaagggcccggcct

SEQ ID NO: 57 - DNA, 93 bp, synthetic construct
  source 1..93: mol_type = other DNA
ttgcggcaaggtctggtgactgatcatcaccggaggaggttcccgccctccccgccccaggggtctccccgctgggtaaaaagggcccggcct

SEQ ID NO: 58 - DNA, 93 bp, synthetic construct
  source 1..93: mol_type = other DNA
ttgcggcaaggtcgggcgactgatcatcgcctgaggaggttcccgccctccccgccccaggggtctccccgctgggtaaaaagggcccggcct

SEQ ID NO: 59 - DNA, 104 bp, synthetic construct
  source 1..104: mol_type = other DNA
aggcaggaggtgaagtcagctgtacccacggctggctgaaaccggggcttgacgaccccccctatccgagttgggcaaggtaacatcacgggtgtgacgacccc

SEQ ID NO: 60 - DNA, 100 bp, synthetic construct
  source 1..100: mol_type = other DNA
ttccggcaaggtgcgccgggggggccttcacgggcccttctagcgcaggggtttgagacaccccccgccccactccttcccagggttggcaacctgggtc

SEQ ID NO: 61 - DNA, 97 bp, synthetic construct
  source 1..97: mol_type = other DNA
gtaaggcaaggtgtgcactggcagccttaacgggctgttggggcgcaggggcttgagcaccccccttccccactcccagcggggcttggcaaccctg

SEQ ID NO: 62 - DNA, 102 bp, synthetic construct
  source 1..102: mol_type = other DNA
gttagcaaggcgctgtcatggtgcttctaacgaggcatgaggcagcgggggcgtgagaaccccccttccccactccggcgtgtagattggcaatcttgcgct

SEQ ID NO: 63 - DNA, 115 bp, synthetic construct
  source 1..115: mol_type = other DNA
acttggcaaggcaccccctggctagtggctgcacttaactgagtggccggctagtcggtgggtgggggcacgtgactcccccttccccacaccggcggcggccgtaaaacgcccc

SEQ ID NO: 64 - DNA, 102 bp, synthetic construct
  source 1..102: mol_type = other DNA
tcctggcaagggctactctgaaagctgctaacgtggtgatcggcgtagcggggcgtgaggaaccccccaccccactcctgggtcagcttggtaactggccca

SEQ ID NO: 65 - DNA, 106 bp, synthetic construct
  source 1..106: mol_type = other DNA
gcgggcagagactgggcctcgctggcgcgtgagctcctgtgggatccggcctgttccatgccacctgttctcgatcagggggaggggatcttaccccctgaactgt

SEQ ID NO: 66 - DNA, 98 bp, synthetic construct
  source 1..98: mol_type = other DNA
gtaaggcaaggtgtgcactggcagccttaacgggctgttgggacacaggggcttgagcaccccccttccccactcccagcggggcttggcaacccccc

SEQ ID NO: 67 - DNA, 89 bp, synthetic construct
  source 1..89: mol_type = other DNA
ttggggcaggcacgcttgcgtgggggagttgcgcccccccccagccagcactcgtcatgatgtgtcggatgccagtgataggcagcctc

SEQ ID NO: 68 - DNA, 64 bp, synthetic construct
  source 1..64: mol_type = other DNA
gcccggcaaggttaacagggggagtagtgccccccccgccccaactcgggtagcgcgtacgctc

SEQ ID NO: 69 - DNA, 72 bp, synthetic construct
  source 1..72: mol_type = other DNA
tccaaggcaacaggcttcggccgggggagtagcgcccccccctttgtgagctcgtaacccccttttggggct

SEQ ID NO: 70 - DNA, 67 bp, synthetic construct
  source 1..67: mol_type = other DNA
acatggcaagggtgcttcggcatgggggagtagcgtccctcccaccccagcgaggctgcaagcctat

SEQ ID NO: 71 - DNA, 71 bp, synthetic construct
  source 1..71: mol_type = other DNA
acctggcaacagccccgtcggggcgggggagtagcgccccccccagtgtgagcgaggtgggaaaccaccta

SEQ ID NO: 72 - DNA, 71 bp, synthetic construct
  source 1..71: mol_type = other DNA
caagggcaaggtgtcttgcgagacaggggcttaacgcacccccccccccagtgaggggggctgatccccca

SEQ ID NO: 73 - DNA, 66 bp, synthetic construct
  source 1..66: mol_type = other DNA
accgggcaagggctcacgcggagtgtgacaagctcccccccccagtccatggccgtggatcggctc

SEQ ID NO: 74 - DNA, 92 bp, synthetic construct
  source 1..92: mol_type = other DNA
gaataggcagggaggagtccaagaaccgtctcggggactctttggggcttgacgaacccccctacccgagtctatattcagtggctggaacc

SEQ ID NO: 75 - DNA, 96 bp, synthetic construct
  source 1..96: mol_type = other DNA
aaaggcagggagaggcttaagaaccgtctcgggagcctcttggggcttgacgaaccccccaacccgagtcaagtccttcaacagtaccgtttcgag

SEQ ID NO: 76 - DNA, 85 bp, synthetic construct
  source 1..85: mol_type = other DNA
tgaaaggggcaagtggccgtataggctggggcgatcgccgtaccccccctttaccaggcgcctcaaccccatgtaccatggggtt

SEQ ID NO: 77 - DNA, 103 bp, synthetic construct
  source 1..103: mol_type = other DNA
acacggcaaggtgctagaagctgaaaccgactcggagctctagcagggggactggcgaccctcccgccccagctggcctctggcagaaacgactcgtgccatt

SEQ ID NO: 78 - DNA, 75 bp, synthetic construct
  source 1..75: mol_type = other DNA
aagaggcaaggggaggcagctagcctccggggcctgacgaccccccttccccagtcattgaaggcaaggggctgc

SEQ ID NO: 79 - DNA, 71 bp, synthetic construct
  source 1..71: mol_type = other DNA
aaagggcaaggcaccacacggctagtgtggtgagggattgacaacccctcctccccagtcggcatgaactt

SEQ ID NO: 80 - DNA, 96 bp, synthetic construct
  source 1..96: mol_type = other DNA
aaagggcaaggcaccacatggaaatgtggtgagggtttgacctcccctcccccccagtcaaccatacataaaacttgaaaaacacatattggtact

SEQ ID NO: 81 - DNA, 70 bp, synthetic construct
  source 1..70: mol_type = other DNA
cagcggcaacaggggagaccccgggcttaacgaccccgccgatgtgagtttggcgaccatggtggatcag

SEQ ID NO: 82 - DNA, 73 bp, synthetic construct
  source 1..73: mol_type = other DNA
caatggcaacagcactctcttaggtgcggggtatggcgaaccccccaatgtgagctcctccccggatggggcg

SEQ ID NO: 83 - DNA, 97 bp, synthetic construct
  source 1..97: mol_type = other DNA
ttcgggcaaggcatgcgagataaaaagggtctcgtatgagggcgtggcaacccctcccccccagctgcggcggcacaaaagcgtctcgcgtgtcgtc

SEQ ID NO: 84 - DNA, 85 bp, synthetic construct
  source 1..85: mol_type = other DNA
tagcggcagggagcagggtagaccaacctgcaggggcttgacgacccccccgtcccgagtcagccaggaggcagaagcgactcgc

SEQ ID NO: 85 - DNA, 106 bp, synthetic construct
  source 1..106: mol_type = other DNA
tgagggcaggataggccaagaatcgtctcgaggctgattggggcttcacgcacccccccatccgagtgcctctcatcttccaaaaccgtctcgggggagatgagac

SEQ ID NO: 86 - DNA, 74 bp, synthetic construct
  source 1..74: mol_type = other DNA
aaagggcagcggtgccggacggcattggggcttggcgaccccccccacgcgagctacccaccattggtgggttc

SEQ ID NO: 87 - DNA, 99 bp, synthetic construct
  source 1..99: mol_type = other DNA
caggggcagaggccggttgattatcattgcagccgtaggggcttggcgacccccccccctcgagccagcctttcaacaaaaccgtctcgggttggaagg

SEQ ID NO: 88 - DNA, 71 bp, synthetic construct
  source 1..71: mol_type = other DNA
acactgcaacaggggaaacccggggatttccgatcccccagatgtgaggaggctggttgcctaacaacctg

SEQ ID NO: 89 - DNA, 77 bp, synthetic construct
  source 1..77: mol_type = other DNA
ctcccgtaaggaaagcgcaagctttgagcattgacaacgctccggccccagtcccccaggttatggaggaataaccc

SEQ ID NO: 90 - DNA, 100 bp, synthetic construct
  source 1..100: mol_type = other DNA
aaggggcaggatatggacagaaccgtctcgggtccagaaggggcttggcgaccccccccatccgagccacccctctaggaagaccgtctcggcctagagg

SEQ ID NO: 91 - DNA, 94 bp, synthetic construct
  source 1..94: mol_type = other DNA
aaggggcagccatgccgcagaaccgtctcgggcggcaaggggcttagcgacccccccctggcgagctgtatgagtgtgataagggcgacatagc

SEQ ID NO: 92 - DNA, 171 bp, synthetic construct
  source 1..171: mol_type = other DNA
aagctgatctccaatgctcttcagtagggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaat

SEQ ID NO: 93 - DNA, 69 bp, synthetic construct
  source 1..69: mol_type = other DNA
cgtttgtgttggtttttatgaccagcttggtacaaaacctgctggtgattttttacccaacaaatatta

SEQ ID NO: 94 - DNA, 68 bp, synthetic construct
  source 1..68: mol_type = other DNA
aagaatatttttaaagacttttttccccaacctctgggttgggttttttctctttaaaatattcaata

SEQ ID NO: 95 - DNA, 86 bp, synthetic construct
  source 1..86: mol_type = other DNA
tgttttgtgttttggctgggtttttccttgttcgcaccggacacctccagtgaccagacggcaaggtttttatcccagtgtatatt

SEQ ID NO: 96 - DNA, 53 bp, synthetic construct
  source 1..53: mol_type = other DNA
attggcagtagagtttttccccagggagcttcactgtctgggttttctctact

SEQ ID NO: 97 - DNA, 53 bp, synthetic construct
  source 1..53: mol_type = other DNA
ttcatcaaggaggttttttcccagcctagctgggttttcctcctttggggaca

SEQ ID NO: 98 - DNA, 52 bp, synthetic construct
  source 1..52: mol_type = other DNA
tttttcgaggaggttttttcctagcaccactaggttttcctcctctgggaac

SEQ ID NO: 99 - DNA, 49 bp, synthetic construct
  source 1..49: mol_type = other DNA
atttactgttggttttcttctctgattttcataagaacttttcccaaca

SEQ ID NO: 100 - DNA, 56 bp, synthetic construct
  source 1..56: mol_type = other DNA
gggtcaggagccccccccctgaacccaggataaccctcaaagtcggggggcaaccc

SEQ ID NO: 101 - DNA, 42 bp, synthetic construct
  source 1..42: mol_type = other DNA
ttgacgcggttctatctagttacgcgttaaaccaactagaaa

SEQ ID NO: 102 - DNA, 53 bp, synthetic construct
  source 1..53: mol_type = other DNA
ccattgccagtagagtttgcatgtctctataaacatgacgttctgactgacta

SEQ ID NO: 103 - DNA, 75 bp, synthetic construct; identical to SEQ ID NO: 6
  source 1..75: mol_type = other DNA